(19) United States
(12) Patent Application Publication
(10) Pub. No.: US 2026/0224605 A1
(43) Pub. Date: Aug. 6, 2026

(54) MODIFIED RNA COMPOSITIONS AND RELATED METHODS

(71) Applicants: BOYCE THOMPSON INSTITUTE FOR PLANT RESEARCH, INC., Ithaca, NY (US); CORNELL UNIVERSITY, Ithaca, NY (US)

(72) Inventors: Frank C. Schroeder, Ithaca, NY (US); Andrew David Lyle Nelson, Ithaca, NY (US); Anna Christine Nelson Dittrich, Ithaca, NY (US); Brian J. Curtis, Ithaca, NY (US); Tyler Jeffrey Schwertfeger, Ithaca, NY (US); Arnaud Tauffenberger, Ithaca, NY (US)

(21) Appl. No.: 19/151,940

(22) PCT Filed: Mar. 1, 2024
(86) PCT No.: PCT/US24/17977

§ 371 Date: Jul. 29, 2025

Related U.S. Application Data
(60) Provisional application No. 63/449,308, filed on Mar. 1, 2023.

(51) Int. Cl. A61K 31/7125; A61K 39/00; A61K 39/12; A61K 48/00; C07H 21/02

(52) U.S. Cl. CPC A61K 31/7125; A61K 39/0011; A61K 39/12; A61K 48/005; C07H 21/02; A61K 2039/53; C12N 2310/336

(57) ABSTRACT

The invention relates to ribonucleotide adducts of novel gluconucleosides and therapeutic compositions containing such adducts as well as methods of using the same.

This application claims priority under 35 U.S.C. § 119 (e) to U.S. Provisional Patent Application No. 63/449,308, filed Mar. 1, 2023. The foregoing application is incorporated by reference herein.

This invention was made with Government support under Grant Nos. R35GM131877 and U2CES030167 awarded by the National Institutes of Health. The United States Government has certain rights in the invention.

### BACKGROUND OF THE INVENTION

Small molecule metabolites derived from plants, fungi and microbes are among the richest sources of therapeutically useful chemical compounds. For example, in the years between 2000 and 2010, approximately 50% of all NCEs (new chemical entities) approved by the US FDA for use as human drugs were natural products or derivatives of natural products (J Nat Prod. 2012 Mar. 23; 75 (3): 311-335).

Recent investigations by the inventors have demonstrated that nematodes are an unexpected and rich source of molecules with diverse biological activities. Meanwhile, as the underlying mechanisms of aging, and a wide range of human health disorders becomes better understood, the need for more selective and efficacious therapeutic and pharmaceutical treatments has never been greater. The present invention addresses these and other related needs.

### FIELD OF THE INVENTION

This invention pertains to the field of small molecule and RNA-based therapeutics and provides therapeutic compositions and pharmacologically active molecules as well as methods of using the same therapeutically.

### SUMMARY OF THE INVENTION

Among other things, the present invention builds upon the inventors' prior discovery of a family of novel gluconucleosides produced by nematodes including C. elegans (doi: 10.1021/jacs. 1c05908). The inventors have now found that certain gluconucleosides are also present in nature covalently linked to RNA. Such adducts likely play a role in the regulation of diverse biochemical and metabolic processes and compositions containing such modified RNA present compelling new options to treat a wide range of health disorders. Additionally, such adducts also presents a new tool by which the stability, activity, bioavailability, biodistribution and/or metabolic fate of RNA or RNA-like therapeutic agents can be modulated. Based on these observations, the inventors have recognized that 1) administering compositions containing gluconucleoside modified RNA (or precursors thereto) provides a useful strategy to treat certain diseases and/or improve the health of animals including humans; and 2) that modifying RNA by covalently linking modular gluconucleosides provides a useful tool to enhance the stability and/or modulate the activity or biodistribution of RNA-based therapeutic agents such as pharmaceuticals, biotherapeutics and vaccines.

For reference, the numbering convention used herein to describe the glucose substitution patterns in MGNs herein is shown below.

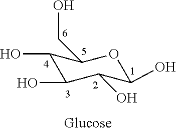 [SMILES: OCC1OC(O)C(O)C(O)C1O]

In most cases, the substituents described herein are attached via covalent bonds to one of the hydroxyl oxygen atoms of the glucose molecule (e.g. through ester, thioester, phosphoester, carbonate, carbamate, thiocarbamate, ether or similar linkages) however, for substituents attached at the 1-position (also referred to as the anomeric position), substituents may either be attached via the oxygen atom, or may be attached via another heteroatom covalently bound to the 1-position—an example of the latter would be an N-linked nucleobase attached to the 1-position.

For reference, the numbering system used herein to refer to RNA, follows the convention of numbering the atoms of the ribose as shown below and of defining 3′ and 5′ ends of linear RNA molecules:

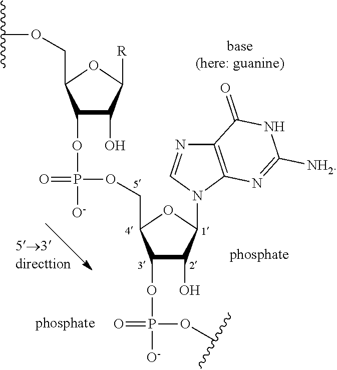

In another aspect, the present invention encompasses methods of improving the health of an animal or of treating or ameliorating a health disorder in an animal by administering to the animal an effective amount of any one or more of the therapeutic compositions described herein. In certain embodiments, the method comprises administering such a composition to a mammal. In certain embodiments, the method comprises administering such a composition to a human.

In another aspect, the present invention encompasses novel compositions of matter including novel molecules and compositions containing such novel molecules. While some of the MGNs and modified RNAs are naturally occurring molecules that have been detected in complex mixtures contained within the bodies of nematodes, pure samples of these molecules and in particular bulk samples of the pure MGNs and modified RNAs free from other biological materials are not found in nature. Additionally, many of the MGNs and modified RNAs described above have not been detected in nature, even with the aid of highly sensitive and selective analytical techniques such as HPLC-coupled high resolution mass spectroscopy. As such, many of the compounds described herein constitute novel compositions of matter.

### Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds of the present invention can comprise one or more asymmetric centers, and thus can exist in various stereoisomeric forms, e.g., enantiomers and/or diastereomers. Thus, inventive compounds and compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of enantiomers or diastereomers are provided.

Furthermore, certain compounds, as described herein may have one or more double bonds that can exist as either a Z or E isomer, unless otherwise indicated. The invention additionally encompasses the compounds as individual isomers substantially free of other isomers and alternatively, as mixtures of various isomers, e.g., racemic mixtures of enantiomers. In addition to the above-mentioned compounds per se, this invention also encompasses compositions comprising one or more compounds.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a compound may, in some embodiments, be provided substantially free of one or more corresponding stereoisomers, and may also be referred to as “stereochemically enriched.”

Where a particular enantiomer is preferred, it may, in some embodiments be provided substantially free of the opposite enantiomer, and may also be referred to as “optically enriched.” “Optically enriched,” as used herein, means that the compound is made up of a significantly greater proportion of one enantiomer. In certain embodiments the compound is made up of at least about 90% by weight of an enantiomer. In some embodiments the compound is made up of at least about 95%, 97%, 98%, 99%, 99.5%, 99.7%, 99.8%, or 99.9% by weight of an enantiomer. In some embodiments the enantiomeric excess of provided compounds is at least about 90%, 95%, 97%, 98%, 99%, 99.5%, 99.7%, 99.8%, or 99.9%. In some embodiments, enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-30 carbon atoms. In certain embodiments, aliphatic groups contain 1-12 carbon atoms. In certain embodiments, aliphatic groups contain 1-8 carbon atoms. In certain embodiments, aliphatic groups contain 1-6 carbon atoms. In some embodiments, aliphatic groups contain 1-5 carbon atoms, in some embodiments, aliphatic groups contain 1-4 carbon atoms, in yet other embodiments aliphatic groups contain 1-3 carbon atoms, and in yet other embodiments aliphatic groups contain 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “unsaturated”, as used herein, means that a moiety has one or more double or triple bonds.

The terms “cycloaliphatic”, “carbocycle”, or “carbocyclic”, used alone or as part of a larger moiety, refer to a saturated or partially unsaturated cyclic aliphatic monocyclic, bicyclic, or polycyclic ring systems, as described herein, having from 3 to 12 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3-6 carbons. The terms “cycloaliphatic”, “carbocycle” or “carbocyclic” also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic groups is bicyclic. In some embodiments, a carbocyclic group is tricyclic. In some embodiments, a carbocyclic group is polycyclic.

The term “alkyl,” as used herein, refers to saturated, straight- or branched-chain hydrocarbon radicals derived from an aliphatic moiety containing between one and six carbon atoms by removal of a single hydrogen atom. Unless otherwise specified, alkyl groups contain 1-12 carbon atoms. In certain embodiments, alkyl groups contain 1-8 carbon atoms. In certain embodiments, alkyl groups contain 1-6 carbon atoms. In some embodiments, alkyl groups contain 1-5 carbon atoms, in some embodiments, alkyl groups contain 1-4 carbon atoms, in yet other embodiments alkyl groups contain 1-3 carbon atoms, and in yet other embodiments alkyl groups contain 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. Unless otherwise specified, alkenyl groups contain 2-12 carbon atoms. In certain embodiments, alkenyl groups contain 2-8 carbon atoms. In certain embodiments, alkenyl groups contain 2-6 carbon atoms. In some embodiments, alkenyl groups contain 2-5 carbon atoms, in some embodiments, alkenyl groups contain 2-4 carbon atoms, in yet other embodiments alkenyl groups contain 2-3 carbon atoms, and in yet other embodiments alkenyl groups contain 2 carbon atoms. Alkenyl groups include, for example, ethenyl, propenyl, butenyl, 1-methyl-2-buten-1-yl, and the like.

The term “alkynyl,” as used herein, refers to a monovalent group derived from a straight- or branched-chain aliphatic moiety having at least one carbon-carbon triple bond by the removal of a single hydrogen atom. Unless otherwise specified, alkynyl groups contain 2-12 carbon atoms. In certain embodiments, alkynyl groups contain 2-8 carbon atoms. In certain embodiments, alkynyl groups contain 2-6 carbon atoms. In some embodiments, alkynyl groups contain 2-5 carbon atoms, in some embodiments, alkynyl groups contain 2-4 carbon atoms, in yet other embodiments alkynyl groups contain 2-3 carbon atoms, and in yet other embodiments alkynyl groups contain 2 carbon atoms. Representative alkynyl groups include, but are not limited to, ethynyl, 2-propynyl (propargyl), 1-propynyl, and the like.

The term “carbocycle” and “carbocyclic ring” as used herein, refers to monocyclic and polycyclic moieties wherein the rings contain only carbon atoms. Unless otherwise specified, carbocycles may be saturated, partially unsaturated or aromatic, and contain 3 to 20 carbon atoms. Representative carbocyles include cyclopropane, cyclobutane, cyclopentane, cyclohexane, bicyclo[2,2,1]heptane, norbornene, phenyl, cyclohexene, naphthalene, spiro[4.5]decane,

The term “aryl” used alone or as part of a larger moiety as in “aralkyl”, “aralkoxy”, or “aryloxyalkyl”, refers to monocyclic and polycyclic ring systems having a total of five to 20 ring members, wherein at least one ring in the system is aromatic and wherein each ring in the system contains three to twelve ring members. The term “aryl” may be used interchangeably with the term “aryl ring”. In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group in which an aromatic ring is fused to one or more additional rings, such as benzofuranyl, indanyl, phthalimidyl, naphthimidyl, phenantriidinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 14 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-14-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle” “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

In some chemical structures herein, substituents are shown attached to a bond which crosses a bond in a ring of the depicted molecule. This means that one or more of the substituents may be attached to the ring at any available position (usually in place of a hydrogen atom of the parent ring structure). In cases where an atom of a ring so substituted has two substitutable positions, two groups may be present on the same ring atom. When more than one substituent is present, each is defined independently of the others, and each may have a different structure. In certain cases where the substituent shown crossing a bond of the ring is —R, this has the same meaning as if the ring were said to be “optionally substituted” as described in the preceding paragraph.

Suitable monovalent substituents on a substitutable carbon atom of an “optionally substituted” group are independently halogen; —(CH₂)₀₋₄R^{o}; —(CH₂)₀₋₄OR^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄₀ (CH₂)₀₋₁Ph which may be substituted with R^{o}; —CH═CHPh, which may be substituted with R^{o}; —NO₂; —CN; —N₃; —) (CH₂)₀₋₄N(R^{o})₂; —(CH₂)₀₋₄N(R^{o}C(O)R^{o}; —) N(R^{o}C(S)R^{o}; —) (CH₂)₀₋₄N(R^{o}C(O)NR^{o} 2; —) N(R^{o}C(S)NR^{o} 2; —) (CH₂)₀₋₄N(R^{o}C(O)OR^{o}; —)) N(R^{o}N(R^{o}C(O)R^{o}); —N(R^{o})N(R^{o})C(O)NR^{o})₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)N(R^{o})₂; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o} 2; —C(S)NR^{o} 2; —C(S)SR^{o}; —SC(S)SR^{o}, —(CH₂)₀₋₄₀C(O)NR^{o} 2; —) C(O) N(OR^{o})R^{o}; —C(O) C(O)R^{o}; —C(O) CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o} 2; —) N(R^{o})S(O)₂R^{o}; —N(OR^{o}) R^{o}; —C(NH) NR^{o} 2; —P(O)₂R^{o}; —P(O)R^{o}₂; —OP(O)R^{o}₂; —OP(O) (OR^{o})₂; SiR^{o}₃; —(C₁₋₄ straight or branched alkylene)O—N(R^{o})₂; or —(C₁₋₄ straight or branched alkylene) C(O)O—N(R^{o})₂, wherein each R^{o} may be substituted as defined below and is independently hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or polycyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₄C(O)N(R^{o})₂; —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, (haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

### BRIEF DESCRIPTION OF THE DRAWINGS

FIG. 1 Shows detection of methyladenine glucoside (maglu #1) and the corresponding 3-phosphate in total RNA obtained from the model organism Caenorhabditis elegans. The digestion protocol involves treatment with three enzymes: nuclease P1, phosphodiesterase from snake venom, and alkaline phosphatase. In brief, 10 μg of RNA was first treated with nuclease P1 (Fisher Scientific) and Phosphodiesterase I from Crotalus adamanteus venom (Sigma) and incubated at 37° C. for 2 hr. Next, RNA was treated with, alkaline phosphatase (Thermo) and incubated to 37° C. for 1 hr. Upon completion, samples were kept on ice until diluted 1/10 (v/v) in HPLC vials using MQ H₂O and stored at −20° C. prior until analysis via HPLC-MS.

FIGS. 2A-2C HPLC-MS analysis (ESI⁺) for gluconucleosides in C. elegans total RNA extracts. FIG. 2A: Analysis for several gluconucleosides using C18 BEH chromatography (Method C′). FIG. 2B Analysis for several gluconucleosides using HILIC chromatography (Method A′). Boxed is the modification of interest, which is derived from N¹-methyladenine based on stable isotope labeling. In addition, co-elution of maglu #1 with the modification from the total RNA extract was observed in 2B in addition to that shown in FIG. 2C. Chromatographic method: A water-methanol gradient was used and a ACQUITY UPLC BEH C18 Column (50 mm×2.1 mm 1.7 μm particle size 130 Å pore size, Waters) which was maintained at 40° C. Solvent A: 0.1% ammonium formate in water; solvent B: methanol. A/B gradient started at 0% B for 3 min, then from 0% to 100% B over 17 min, held at 100% B for 2 min, then rapidly down to 0% B over 1 min, and held for 2 min to equilibrate the column.

FIGS. 3A-3B. Identification of N¹-methyladenine glucoside in C. elegans RNA extracts. FIG. 3A: Analysis for m/z=312.1302 (HILIC-MS, ESI⁺, Method A′) for total RNA with full enzymatic treatment, C. elegans endo-metabolome extract, and synthetic maglu #1. In addition, an EIC for m/z=315.1490 is displayed, for a sample derived from total RNA, after full enzymatic treatment, obtained from C. elegans supplemented with CD3-N¹-methyladenine. FIG. 3B: EICs for m/z=392.0966 (HILIC-MS, ESI⁺, Method A′) for total RNA, enzymatically digested but without phosphatase treatment, C. elegans endo-metabolome extract, and synthetic maglu #2. In addition, a trace for total RNA, without phosphatase treatment, extracted from C. elegans treated with CD3-N¹-methyladenine for m/z=395.1154 is displayed.

FIGS. 4A-4B Shows MS² analysis for N¹-methyladenine glucoside identified in RNA extracts. FIG. 4A: MS² spectra for m/z=312.1302 and 315.1491 corresponding to peaks shown in FIG. 2A following total enzymatic treatment. FIG. 4B: MS² spectra for m/z=392.0966 and 395.1154 corresponding to peaks shown in FIG. 2B following enzymatic treatment with no phosphatase step. Note that as signal intensity was low, similar masses to the parent ion were also observed at similar signal intensity; thus, the parent masses were not displayed in the spectrum.

FIG. 5. Demonstration of the presence of maglu #1 in mRNA. HPLC-MS analysis in positive ionization mode, showing ion chromatogram for m/z 312.1293, corresponding to maglu #1, for a sample of enzymatically hydrolyzed C. elegans mRNA, isolated using the Dynabeads mRNA Purification Kit.

### DETAILED DESCRIPTION OF THE INVENTION

### I. Therapeutic Compositions

In one aspect, the present invention encompasses therapeutic compositions. In certain embodiments, such compositions comprise an effective amount of RNA containing one or more covalently linked gluconucleosides. In certain embodiments, such therapeutic compositions comprise RNA incorporating compounds known as Modular Gluconucleosides or MGNs—a family of small molecules newly identified in nematodes. The natural MGNs the inventors have thus far identified from C. elegans each contain a glucose moiety covalently bound to a nucleobase via an N-glycoside linkage. In addition to therapeutic compositions based on RNA-adducts of such naturally-occuring MGNs, the present invention also provides artificial MGNs containing other nucleobases (as well as derivatives, anaologs or mimics of nucleobases); MGNs containing other patterns of nucleobase attachment (e.g. via a covalent linkage to a different atom of the nucleobase); MGNs containing other glycosidic linkages (e.g. attachment of the nucleobase via a intervening linker or via a connection other than an N-glycosidic linkage); and/or MGNs containing other substitution patterns on the sugar moiety (e.g. through variation of the substituents on the glucose, or via incorporation of pyranosides other than glucose, for example pyranoses derived from other hexoses, e.g. galactose or mannose, or derived from pentoses, e.g. xylose) as well as RNA adducts of such artificial MGNs.

In one aspect, the present invention provides therapeutic compositions comprising a MGN bound to one or two ribonucleotides. In certain embodiments, such ribonucleotides are part of a larger oligo- or polynucleotide and the composition comprises an MGN adduct of RNA. In certain embodiments, the invention encompasses compositions comprising MGN-adducts of RNA having a specific biological function. For example:

In certain embodiments, compositions comprise MGN adducts of RNA involved in protein synthesis including, but not limited to: MGN-modified adducts of: Messenger RNA (mRNA), Transfer RNA (tRNA), Transfer Messenger RNA (tmRNA). Ribosomal RNA (rRNA), or Signal Recognition Particle RNA (SRP RNA).

In certain embodiments, the invention provides compositions comprising MGN-adducts of RNA involved in post translational modification or in DNA replication including, but not limited to MGN modified adducts of: Ribonuclease P (RNase P), Ribonuclease MRP (RNase MRP), Y RNA, Telomerase RNA Component (TREC), Guide RNA (gRNA), Spliced Leader RNA (SL RNA), Small Nuclear RNA (snRNA), Small Nucleolar RNA (snoRNA), SmY RNA, or Small Cajal body-specific RNA (scaRNA).

In certain embodiments, the invention provides compositions comprising MGN-adducts of regulatory RNAs including, but not limited to, MGN modified adducts of: Antisense RNA (aRNA or asRNA), Enchancing RNA (eRNA), Long Noncoding RNA (lncRNA) Micro RNA (miRNA), Piwi Interacting RNA (piRNA), CRISPR RNA (crRNA), Short Hairpin RNA (shRNA), and 7SK RNA.

In certain embodiments, the invention provides compositions comprising MGN-adducts of viral RNA or fragments of viral RNA. In certain embodiments, the invention provides MGN adducts of RNA having utility in the formulation of vaccine compositions.

In certain embodiments, the invention provides compositions comprising MGN-adducts of artificial RNA constructs, for example RNA constructs or derivatives having utility as therapeutic agents or having utility in research applications.

MGNs may be bound to RNA through a covalent bond to any suitable atom of the MGN molecule having sufficient valency to form such a bond. In certain embodiments, the linkage of the MGN to the RNA is through a phosphate linkage (e.g. (MGN-OP(O)OX)O-RNA, where MGN is the modular gluconucleoside, X is —H, any metal atom, or a negative charge, and RNA is any oligoribonucleotide or RNA molecule) or through a diphosphate (e.g MGN-O [P(O)OX) O]₂-RNA), triphosphate (e.g MGN-O [P(O)OX) O]₃-RNA), or oligophosphate (MGN-O [P(O)OX)O]ₙ-RNA, with n=4-7) linkage. In certain embodiments, the linkage of the MGN to the RNA is through a phosphate, diphosphate, triphosphate, or oligophosphate linkage to one or both of the 3- and 6-positions of the MGN glucose moiety. In certain embodiments, the linkage of the MGN to the RNA is through a phosphate, diphosphate, triphosphate, or oligophosphate linkage to the 3-position of the MGN glucose moiety. In certain embodiments, the linkage of the MGN to the RNA is through a phosphate, diphosphate, triphosphate, or oligophosphate linkage to the 6-position of the MGN glucose moiety. In certain embodiments, the MGN is linked to two RNA molecules through a phosphate, diphosphate, triphosphate, or oligophosphate linkage to both the 3- and the 6-positions of the MGN glucose moiety.

MGNs may be bound to RNA through a covalent bond to any suitable atom of the RNA molecule having sufficient valency to form such a bond. In certain embodiments, the MGN is covalently bound to a terminal ribonucleotide of the RNA. In certain embodiments, the MGN is bound to the 5′ end of the RNA. In certain embodiments, the MGN is bound to the 3′ end of the RNA. In certain embodiments, the MGN is bound to the 5′ end of the RNA through a phosphate (or di- or tri- or oligo-phosphate) linkage formed between the 5′ position of the ribose of the terminal ribonucleotide of the RNA and the 3-position of the MGN glucose moiety. In certain embodiments, the MGN is bound to the 3′ end of the RNA through a phosphate (or di- or tri- or oligo-phosphate) linkage formed between the 6-position of the MGN glucose moiety and the 3′ position of the ribose moiety of the terminal ribonucleotide. In certain embodiments, the MGN is bound to the 5′ end of the RNA through a phosphate (or di- or tri- or oligo-phosphate) linkage between the 5′ position of the ribose of the terminal ribonucleotide of the RNA and the 6-position of the MGN glucose moiety. In certain embodiments, the MGN is bound to the 3′ end of the RNA through a phosphate (or di- or tri- or oligo-phosphate) linkage formed between the 6-position of the MGN glucose moiety to the 3′ position of the terminal ribose on the RNA. In certain embodiments, the MGN is bound to the 5′ or the 3′ end of the RNA through a phosphate (or di- or tri- or oligo-phosphate) linkage between the 2′ position of the ribose of a terminal ribonucleotide and the MGN. In certain embodiments, the MGN is bound to the RNA through a phosphate diester linkage between the 2′ position of the ribose of a terminal ribonucleotide and the 3 position of the MGN glucose moiety. In certain embodiments, the MGN is bound to the RNA through a phosphate diester linkage between the 2′ position of the ribose of a terminal ribonucleotide and the 6 position of the MGN glucose moiety.

In certain embodiments, provided compositions comprise RNA where the MGN is bound via a covalent linkage to a non-terminal ribonucleotide. In certain embodiments, an MGN is bound via a phosphate linkage to the 2′ position of a ribonucleotide. In certain embodiments, the MGN is bound to the RNA via a phosphate (or di- or tri- or oligo-phosphate) linkage between the 2′ position of the ribose of a non-terminal ribonucleotide and the 3-position of the MGN glucose moiety. In certain embodiments, the MGN is bound to the RNA via a phosphate (or di- or tri- or oligo-phosphate) linkage between the 2′ position of the ribose of a non-terminal ribonucleotide and the 6-position of the MGN glucose moiety.

In certain embodiments, where provided compositions comprise an MGN bound via a covalent linkage to a non-terminal ribonucleotide, the MGN takes the place of a ribonucleotide in an RNA chain, or acts as a bridge to link two RNA chains (which may be the same or different). In certain such embodiments, the MGN is covalently linked to RNA at both the 3- and 6-positions of the glucose composing the MGN. In certain such embodiments, the MGN is bound to the RNA by phosphate (or di- or tri- or oligo-phosphate) linkages to 3- and 6-positions of the glucose composing the MGN. In certain such embodiments, the MGN is bound to the 5′ position of a ribonucleotide via a phosphate (or di- or triphosphate) linkage to the 3 position of the MGN glucose moiety. In certain such embodiments, the MGN is bound to the 3′ position of a ribonucleotide via a phosphate (or di- or triphosphate) linkage to the 6 position of the MGN glucose moiety. In certain embodiments, the MGN replaces a ribonucleotide in the RNA chain and is enchained therein via linkages between the 3′ position of a ribonucleotide via a phosphate (or di- or tri- or oligo-phosphate) linkage to the 6 position of the MGN and between the 5′ position of another adjacent ribonucleotide via a phosphate (or di- or tri- or oligo-phosphate) linkage to the 3 position of the MGN glucose moiety.

In certain embodiments, provided therapeutic compositions comprise one or more compounds conforming to Formula I:

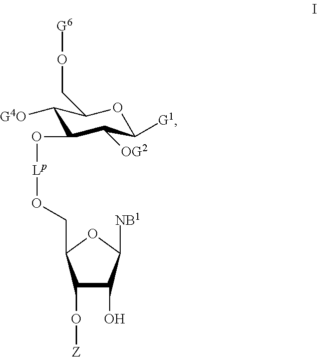

or a pharmaceutically acceptable salt thereof, wherein:
G¹ is —OR¹⁰, —OC(O)R¹¹, or an optionally substituted group selected from the group consisting of 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur;
G² is selected from —H, —C(O)R, —C(S)R, —C(O)OR, —C(O)SR, —C(S)OR, —C(O)N(R)₂, —C(S)N(R)₂, or an optionally substituted group selected from C₁₋₃₂ aliphatic, C₁-32 heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
G⁴ is selected from —H, —C(O)R, —C(S)R, —C(O)OR, —C(O)SR, —C(S)OR, —C(O)N(R)₂, —C(S)N(R)₂, or an optionally substituted group selected from C₁₋₃₂ aliphatic, C₁-32 heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
G⁶ is selected from —H, —C(O)R, —C(S)R, —C(O)OR, —C(O)SR, —C(S)OR, —C(O)N(R)₂, —C(S)N(R)₂, or optionally substituted phosphate, diphosphate, triphosphate, or oligophosphate; a phosphate-, diphosphate-, triphosphate-, or oligophosphate-linked ribonucleotide; a phosphate-, diphosphate-, triphosphate-, or oligophosphate-linked RNA molecule; an acyl-linked amino acid, or peptide, or an optionally substituted group selected from C₁₋₃₂ aliphatic, C₁₋₃₂ heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
L^{P} is selected from:

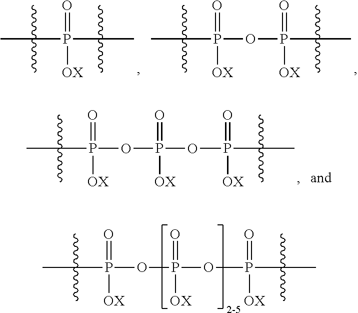

where X is independently selected at each occurrence from —H, C₁₋₄ aliphatic, a negative charge, and any metal atom or ion;
R¹⁰ and R¹¹ are each independently an optionally substituted group selected from the group consisting of: C₁₋₃₂ aliphatic, C₁₋₃₂ heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
each R is independently-H or an optionally substituted group selected from C₁₋₆ aliphatic, C₁₋₆ heteroaliphatic, 3- to 7-membered monocyclic cycloaliphatic, 3- to 7-membered saturated or partially unsaturated monocyclic heterocyclyl having 1-2 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or
two R, when attached to the same nitrogen atom, are taken together to form an optionally substituted 3- to 7-membered saturated or partially unsaturated ring having 0-1 additional heteroatoms independently selected from nitrogen, oxygen, and sulfur
NB¹ is any nucleobase, including nucleobases that occur in RNA (e.g., any type of RNA from any organism), as well as non-natural nucleobase mimics and derivatives; and
Z is selected from —H; optionally substituted phosphate, diphosphate, or triphosphate; a ribonucleotide, and an RNA molecule.

In certain embodiments, provided therapeutic compositions comprise one or more compounds conforming to any of the formulae in Table 1:

TABLE 1

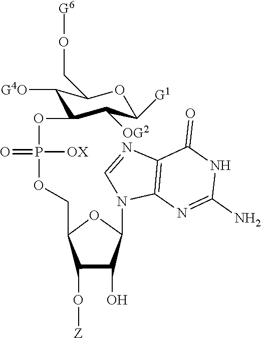

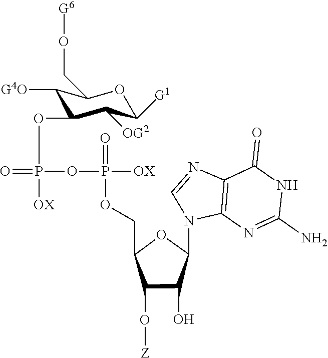

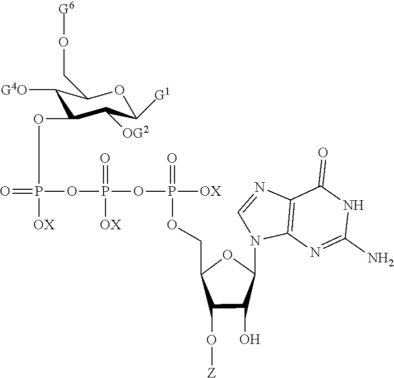

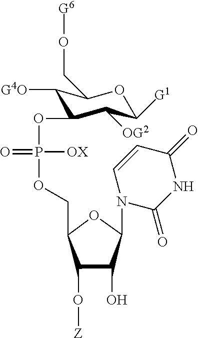

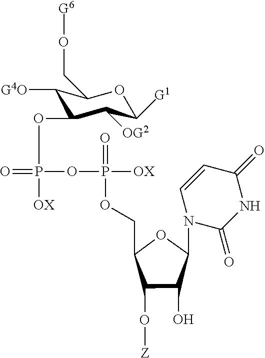

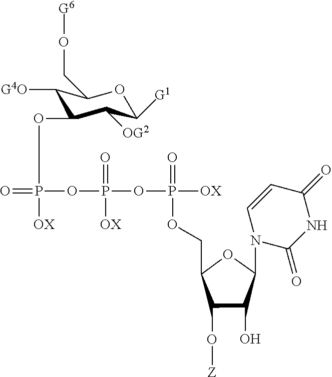

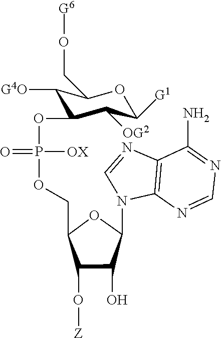

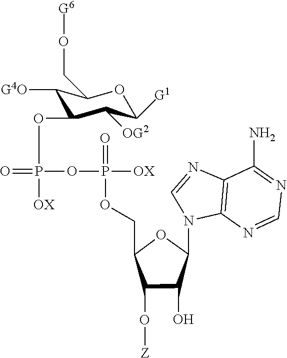

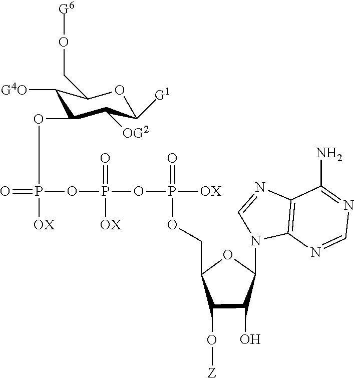

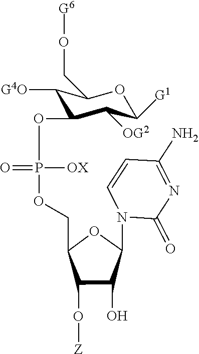

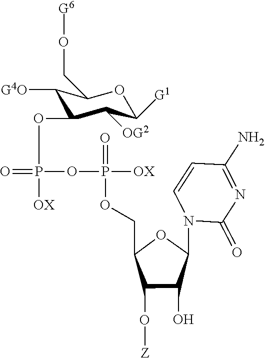

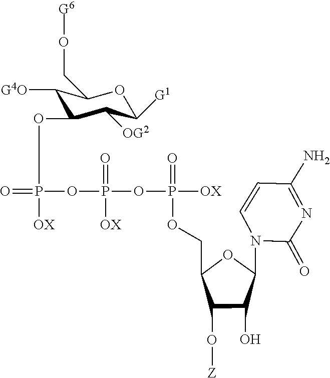

or a pharmaceutically acceptable salt thereof, where each of G¹, G², G⁴, G⁶, Z, and X is as defined above and in the genera and subgenera herein.

In certain embodiments, provided therapeutic compositions comprise one or more compounds conforming to Formula II:

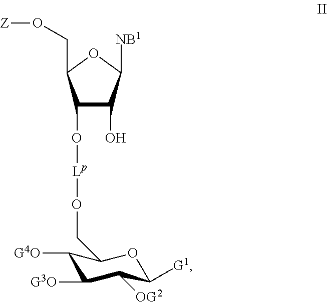

or a pharmaceutically acceptable salt thereof, wherein:
each of G¹, G², G⁴, L^{P}, NB¹, and Z is as defined above and in the genera and subgenera herein;
G³ is selected from —H, —C(O)R, or optionally substituted phosphate, diphosphate, triphosphate, or oligophosphate; a phosphate-, diphosphate-, triphosphate, or oligophosphate-linked ribonucleotide; a phosphate-, diphosphate-, triphosphate-, or oligophosphate linked RNA molecule; an acyl-linked amino acid, or an optionally substituted group selected from C₁₋₃₂ aliphatic, C₁₋₃₂ heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; and each R is independently-H or an optionally substituted group selected from C₁₋₆ aliphatic, C₁₋₆ heteroaliphatic, 3- to 7-membered monocyclic cycloaliphatic, 3- to 7-membered saturated or partially unsaturated monocyclic heterocyclyl having 1-2 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or
two R, when attached to the same nitrogen atom, are taken together to form an optionally substituted 3- to 7-membered saturated or partially unsaturated ring having 0-1 additional heteroatoms independently selected from nitrogen, oxygen, and sulfur.

In certain embodiments, provided therapeutic compositions comprise one or more compounds conforming to any of the formulae in Table 2:

TABLE 2

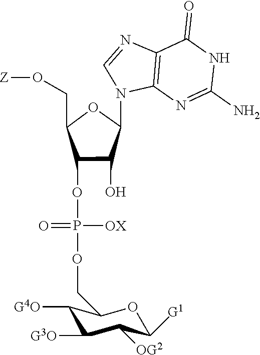

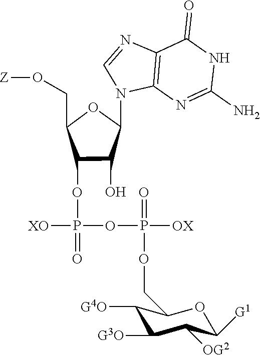

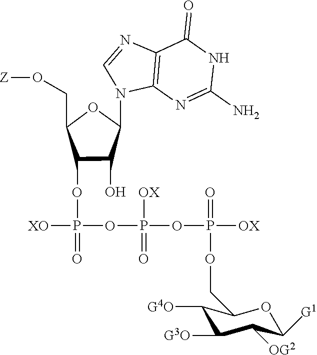

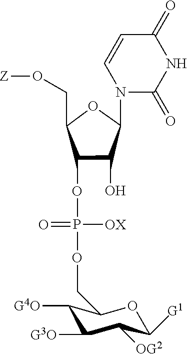

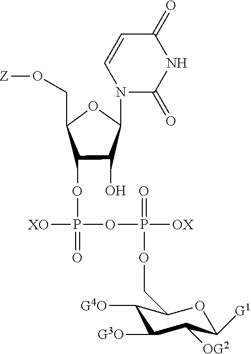

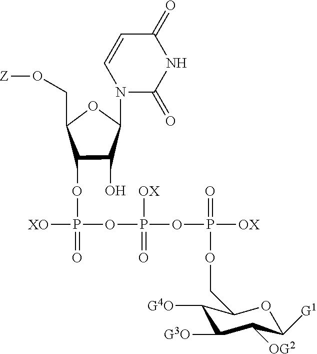

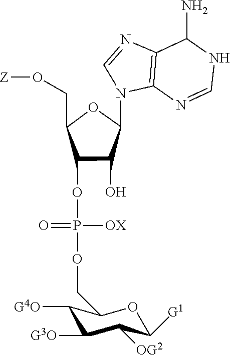

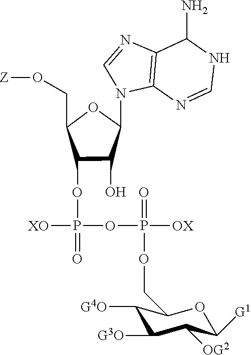

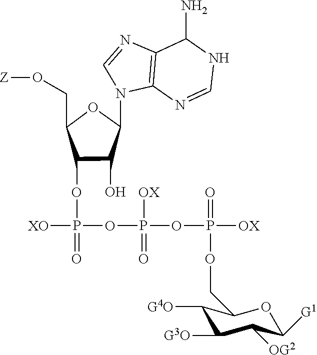

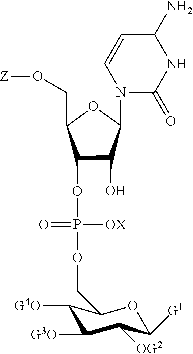

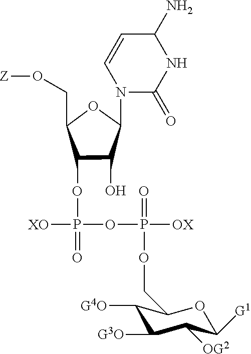

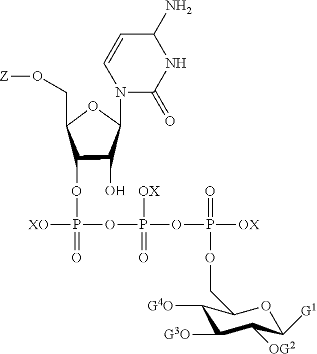

or a pharmaceutically acceptable salt thereof, where each of G¹, G², G³, G⁶, Z, and X is as defined above and in the genera and subgenera herein.

In certain embodiments, provided therapeutic compositions comprise one or more compounds conforming to Formula III:

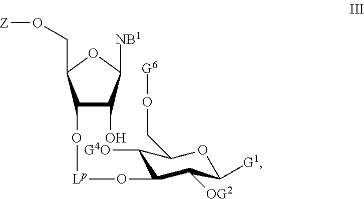

or a pharmaceutically acceptable salt thereof, wherein:
each of G¹, G², G⁴, G⁶, L^{P}, NB¹, and Z is as defined above and in the genera and subgenera herein.

In certain embodiments, provided therapeutic compositions comprise one or more compounds conforming to any of the formulae in Table 3:

TABLE 3

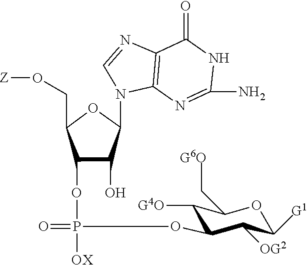

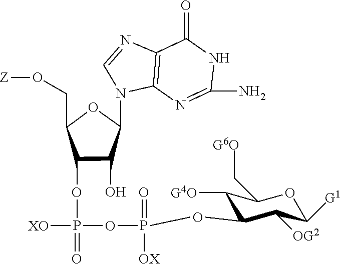

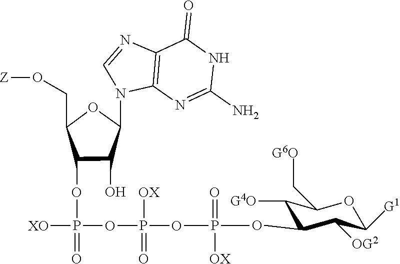

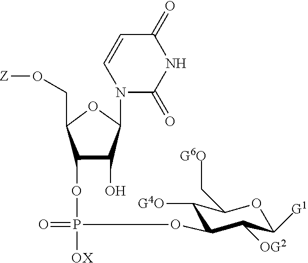

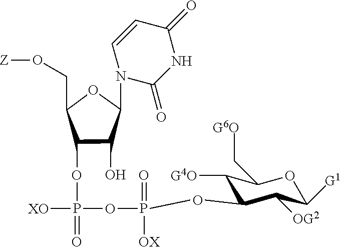

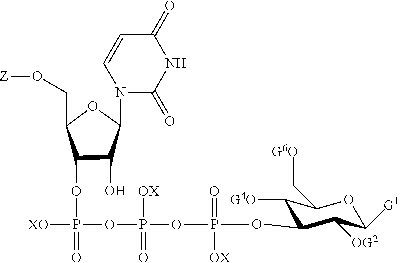

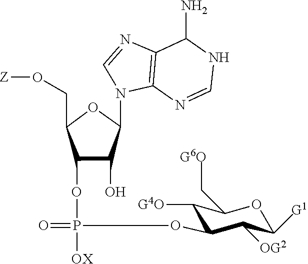

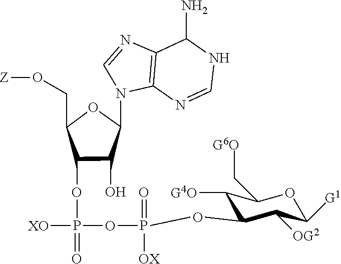

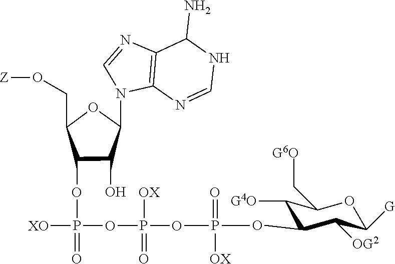

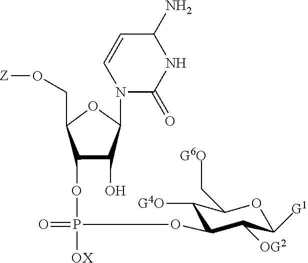

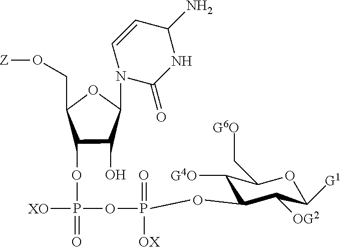

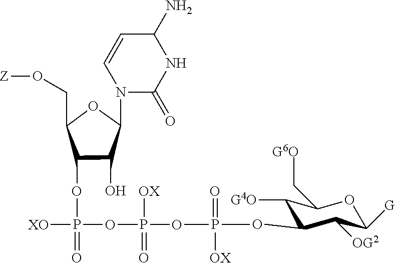

or a pharmaceutically acceptable salt thereof, where each of G¹, G², G⁴, G⁶, X, and Z is as defined above and in the genera and subgenera herein.

In certain embodiments, provided therapeutic compositions comprise one or more compounds conforming to Formula IV:

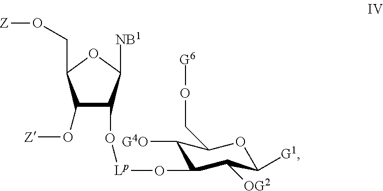

wherein:
or a pharmaceutically acceptable salt thereof, each of G¹, G², G⁴, G⁶, L^{P}, NB¹, and Z is as defined above and in the genera and subgenera herein; and
Z′ is selected from —H; optionally substituted phosphate, diphosphate, triphosphate, or oligophosphate; a ribonucleotide, and an RNA molecule.

In certain embodiments, provided therapeutic compositions comprise one or more compounds conforming to any of the formulae in Table 4:

TABLE 4

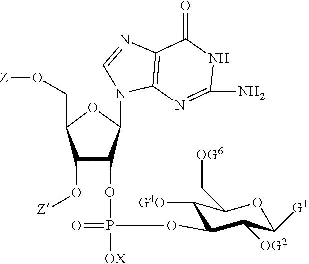

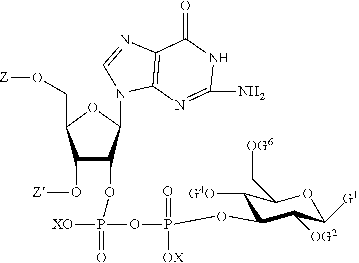

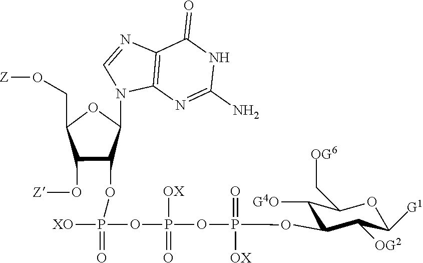

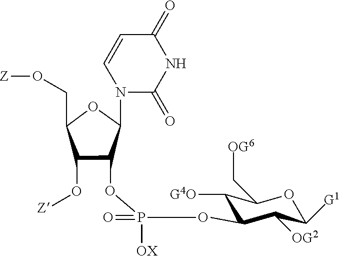

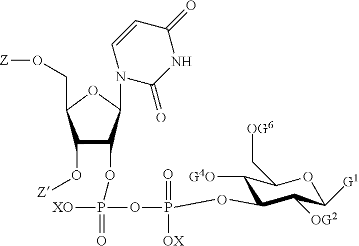

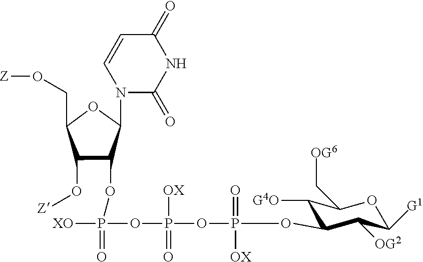

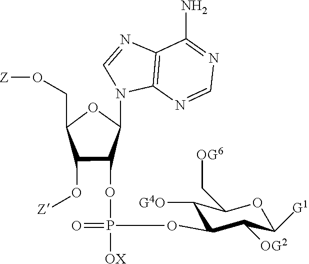

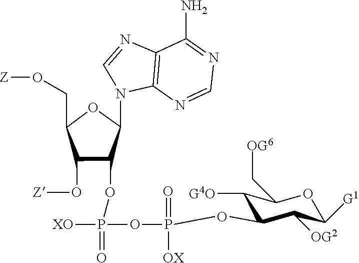

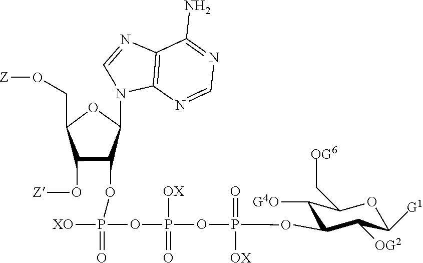

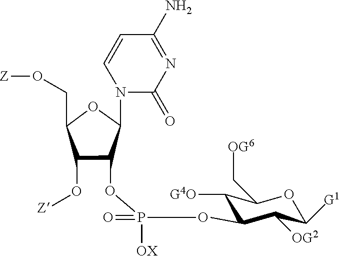

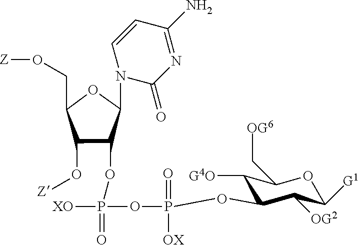

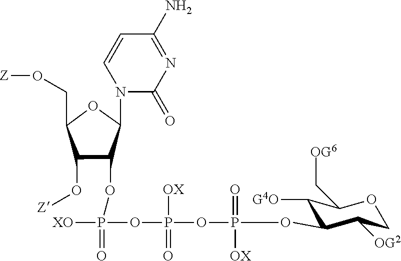

or a pharmaceutically acceptable salt thereof, where each of G¹, G², G⁴, G⁶, X, Z, and Z′ is as defined above and in the genera and subgenera herein.

In certain embodiments, provided therapeutic compositions comprise one or more compounds conforming to Formula V:

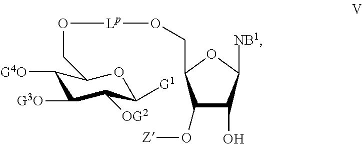

or a pharmaceutically acceptable salt thereof, wherein:
each of G¹, G², G³, G⁴, L^{P}, NB¹, and Z′ is as defined above and in the genera and subgenera herein.

In certain embodiments, provided therapeutic compositions comprise one or more compounds conforming to any of the formulae in Table 5:

TABLE 5

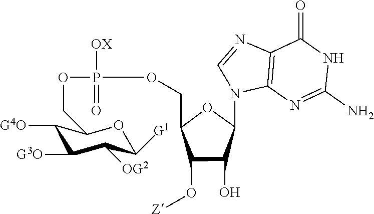

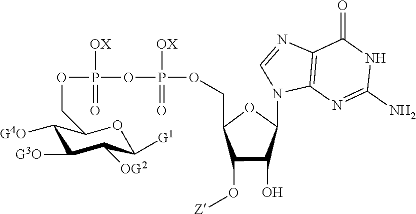

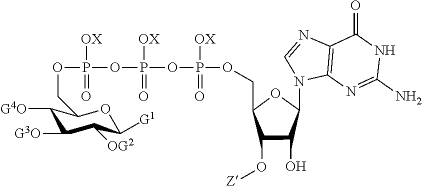

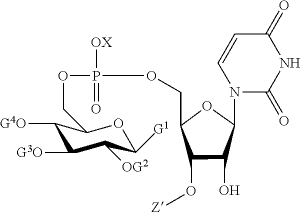

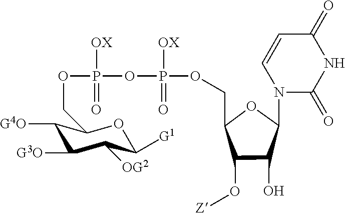

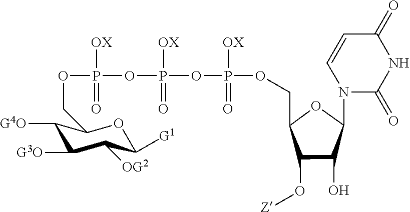

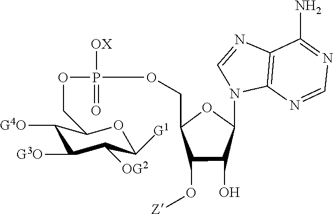

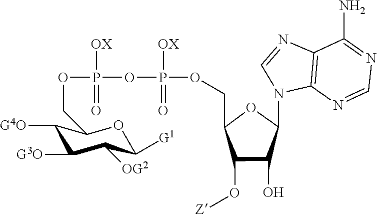

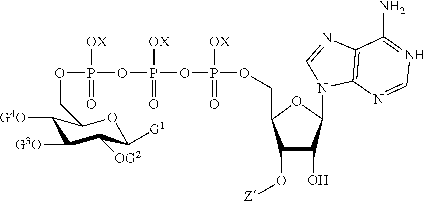

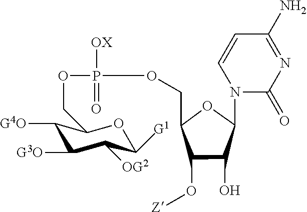

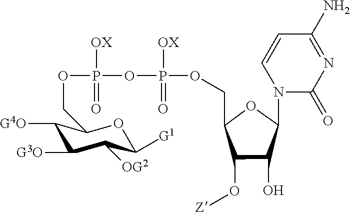

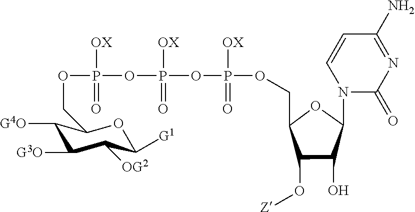

or a pharmaceutically acceptable salt thereof, where each of each of G¹, G², G³, G⁴, X, and Z, is as defined above and in the genera and subgenera herein.

In certain embodiments, provided therapeutic compositions comprise one or more compounds conforming to Formula VI:

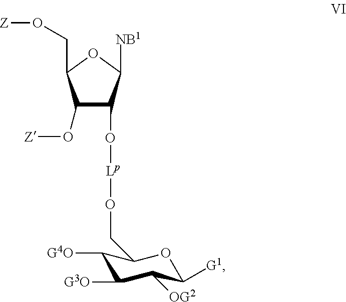

or a pharmaceutically acceptable salt thereof, wherein:
each of G¹, G², G³, G⁴, L^{P}, NB¹, Z, and Z′ is as defined above and in the genera and subgenera herein.

In certain embodiments, provided therapeutic compositions comprise one or more compounds conforming to any of the formulae in Table 6:

TABLE 6

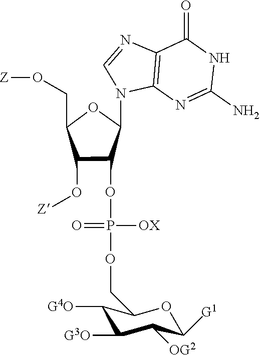

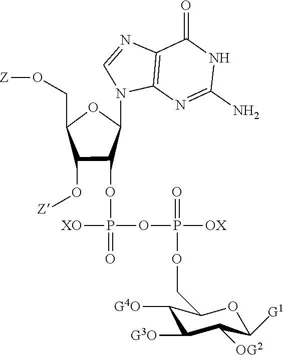

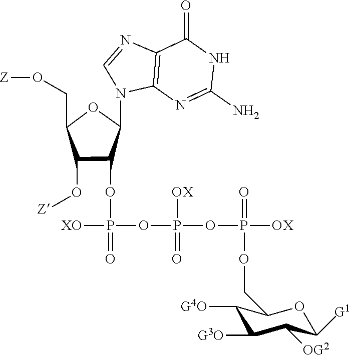

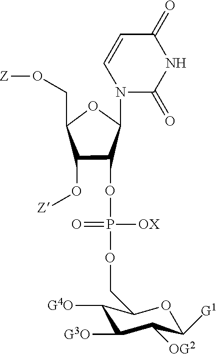

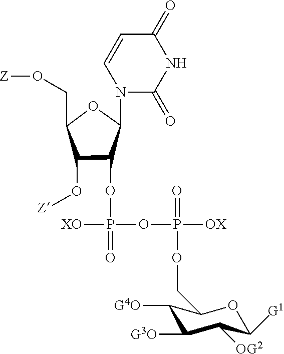

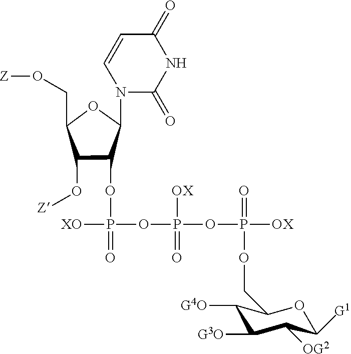

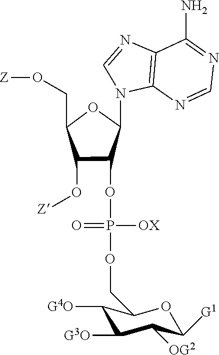

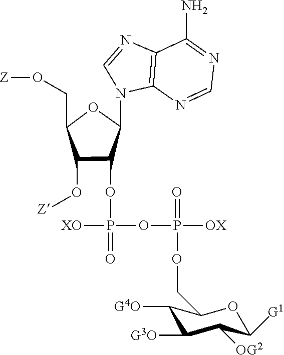

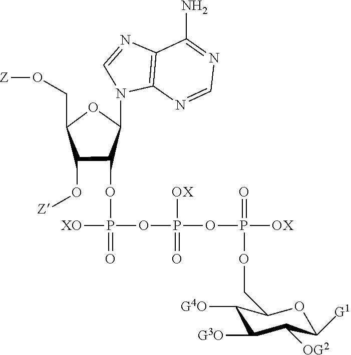

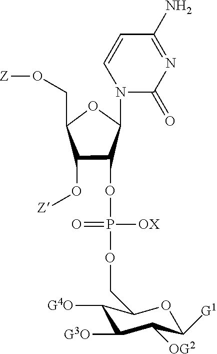

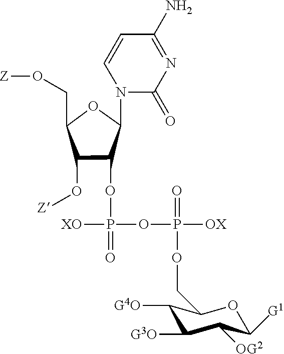

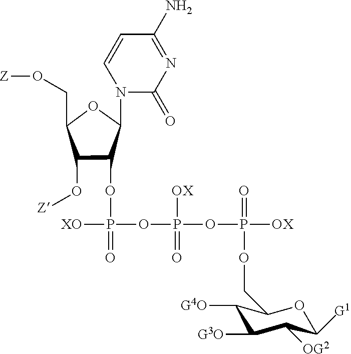

or a pharmaceutically acceptable salt thereof, where each of G¹, G², G³, G⁴, X, and Z′ is as defined above and in the genera and subgenera herein.

In certain embodiments, G¹ in any of the formulae herein comprises a nucleobase, or a derivative or precursor of a nucleobase linked to the 1-position of the glucose through any suitable atom.

In certain embodiments, the nucleobase is linked to the glucose through a nitrogen or oxygen atom comprising part of the nucleobase structure. In certain embodiments, the nucleobase is N-linked.

In certain embodiments, the moiety G¹ is selected from the group consisting of:

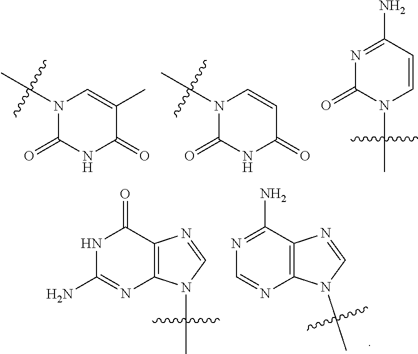

In certain embodiments, the moiety G¹ is selected from the group consisting of:

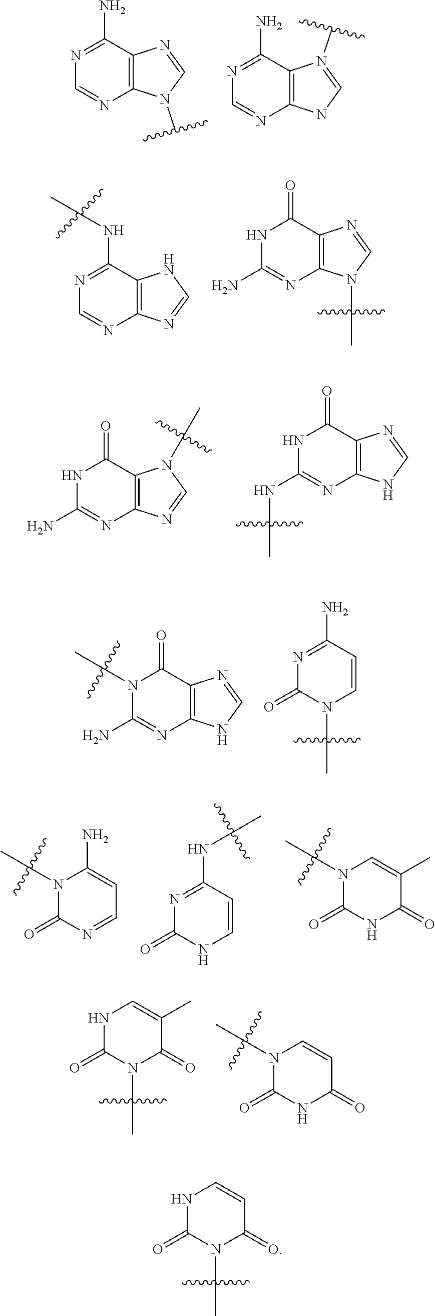

In certain embodiments, the moiety G¹ is selected from the group consisting of:

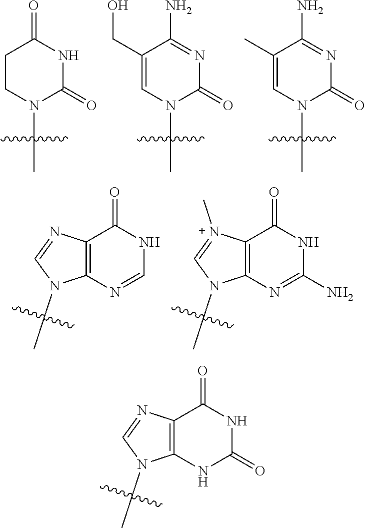

In certain embodiments, the moiety G¹ is selected from the group consisting of:

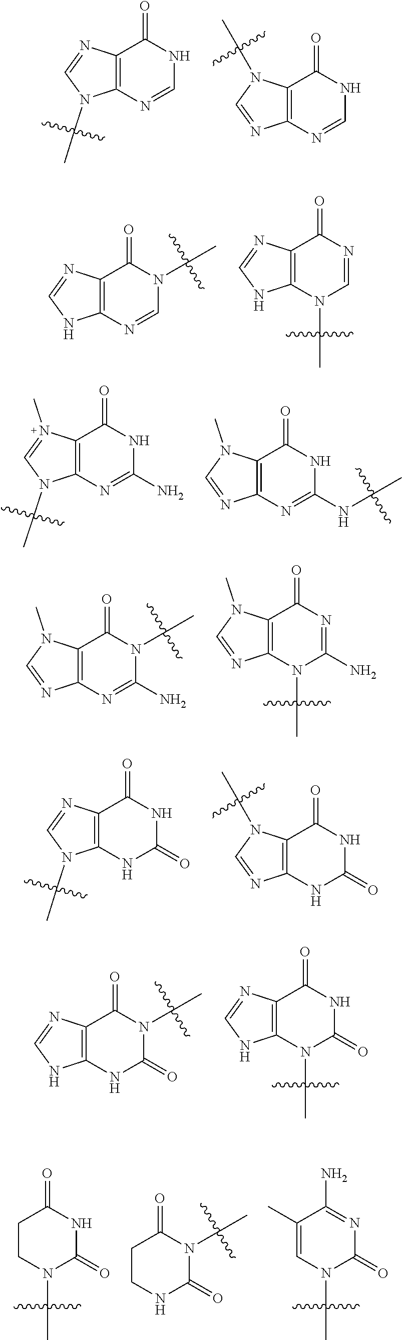

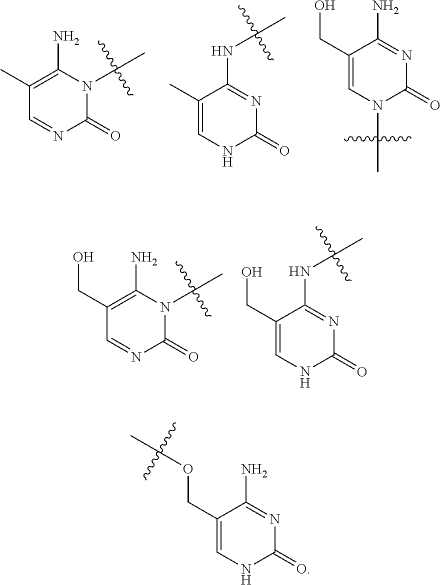

In certain embodiments, the moiety G¹ is selected from the group consisting of:

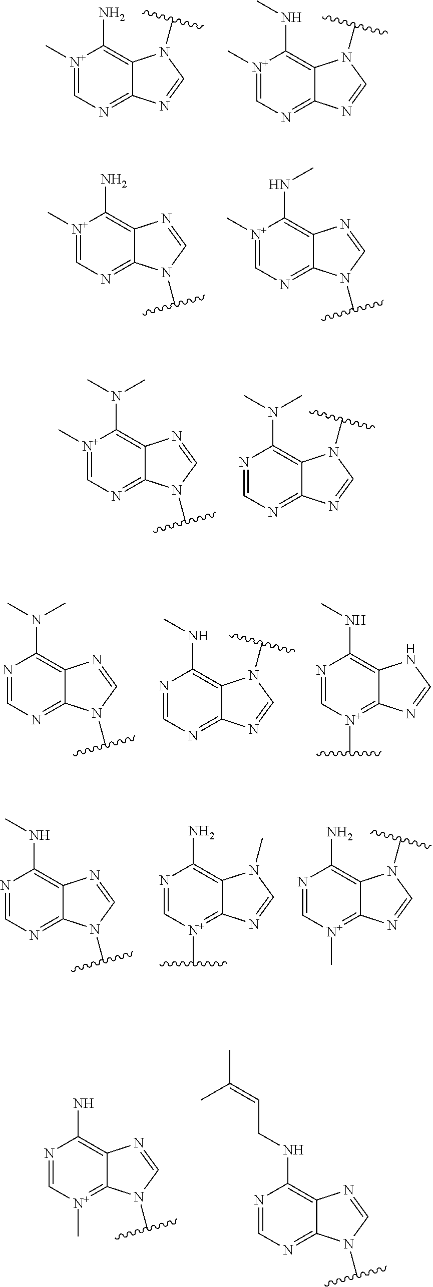

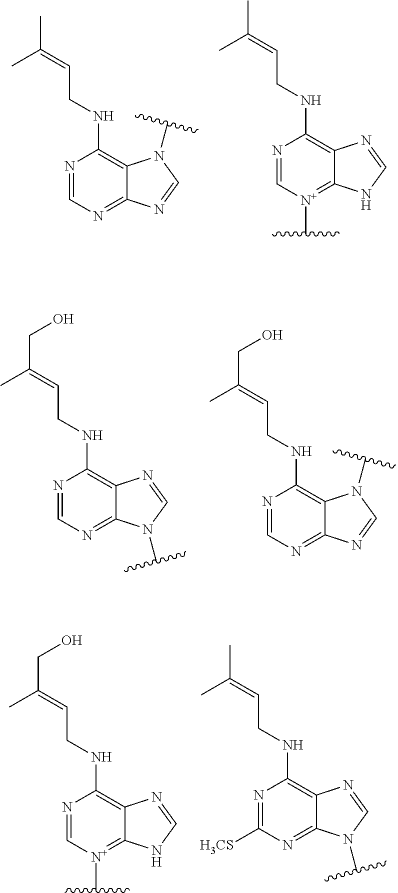

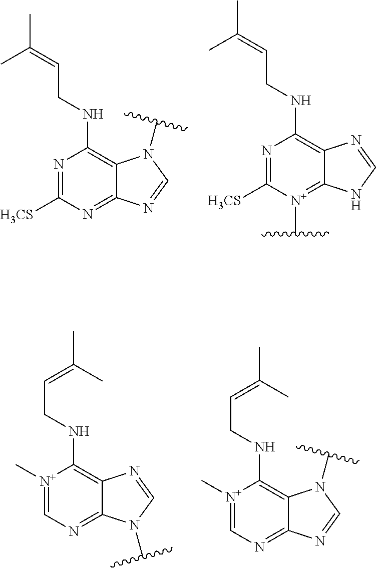

In certain embodiments, the moiety G¹ is selected from the group consisting of:

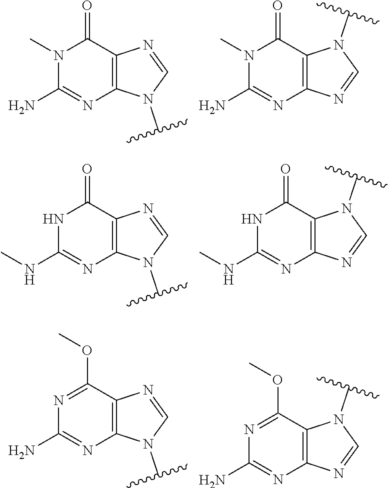

In some embodiments, at least one of G² and G⁴ is —H.

In some embodiments, G⁶ is other than-H.

In some embodiments, —Z is other than-H. In some embodiments, —Z is a RNA molecule (e.g., an oligonucleotide or polynucleotide).

### II. Therapeutic Methods

In another aspect, the present invention encompasses methods of improving the health of an animal or of curing or ameliorating a health disorder in an animal by administering to the animal an effective amount of any one or more of the therapeutic compositions described above. In certain embodiments, the method comprises administering such a composition to a mammal. In certain embodiments, the method comprises administering such a composition to a human.

In another aspect, the present invention encompasses methods of improving the health of an animal or of treating or ameliorating a health disorder in an animal by administering to the animal an effective amount of any one or more of the therapeutic compositions described above. In certain embodiments, the method comprises administering such a composition to a mammal. In certain embodiments, the method comprises administering such a composition to a human.

In one aspect, the present invention comprises methods for improving the efficacy of RNA-based vaccines, for example mRNA-based vaccines. RNA vaccines can be used in both infection therapy (e.g. against virus-caused infections, such as Covid-19) and cancer immunotherapy. In one aspect, efficacy of such vaccines can be improved by increasing stability, for example as a result of increased resistance to endogenous oligonucleotide-degrading enzymes. In some embodiments, increased stability can be achieved by attaching a MGN to mRNA or mRNA-derivatives as they are typically used in mRNA-based vaccines. In certain such embodiments, the MGN is attached to the 5′- or 3′-ends of mRNA or and mRNA-derivative. In other embodiments, one or more MGNs are attached to the 2′-position of the mRNA or mRNA derivatives.

In another aspect, the current invention comprises methods to increase the stability of mRNA or mRNA-derivatives for gene therapy. Genetic and rare diseases (GARDs) affect more than 350 million patients worldwide and remain a significant challenge to medicine. RNA-based therapeutics, such as RNA interference (RNAi), messenger RNA (mRNA), long non-coding RNA (lncRNA), and RNA-involved genome editing technologies demonstrate great potential as a therapy tool for treating genetic associated rare diseases. In one aspect, MGNs can be used to increase stability and resistance to endogenous degradation of mRNA, siRNA, lncRNA, and miRNA. For example, MGN-modified siRNA can be used to treat the inherited skin disorder, pachyonychia congenital, or polyneuropathy in people with hereditary transthyretin-mediated amyloidosis, a fatal rare disease. Additional examples for potential uses of MGN-modified siRNAs are included by reference to Table 1 in Zhao W, Hou X, Vick O G, Dong Y. RNA delivery biomaterials for the treatment of genetic and rare diseases. Biomaterials. 2019 October; 217:119291. doi: 10.1016/j.biomaterials.2019.119291.

In another aspect, the current invention relates to the use of MGN-modified mRNA derivatives as a treatment to effectuate endogenous production of desired functional proteins in the context of protein replacement therapies (PRTs). Many genetic and rare diseases are characterized by protein deficiencies or malfunctions that can be treated with PRTs. Here, treatment with mRNA instead of supplementation of the desired protein is advantageous and more effective because one mRNA molecule can generate multiple copies of a protein. In some applications, MGN-modified mRNA can provide better translatability, stability, immunogenicity, and intracellular delivery. Examples for the potential use of MGN-modified mRNAs are included by reference to Table 2 in Zhao W, Hou X, Vick O G, Dong Y. RNA delivery biomaterials for the treatment of genetic and rare diseases. Biomaterials. 2019 October; 217:119291. doi: 10.1016/j.biomaterials.2019.119291.

In another aspect, the current invention relates to using MGN modification to increase stability and efficacy of RNA or DNA employed for gene editing systems. This includes genome editing tools such as meganucleases, zinc finger nucleases (ZFNs), transcription activator-like effector nucleases (TALENs), clustered regularly interspaced short palindromic repeats-associated protein 9 (CRISPR-Cas9), CRISPR from Prevotella and Francisella 1 (CRISPR-Cpf1, CRISPR-Cas12a), and many others that can be used for gene engineering to potentially correct disease-causing mutations. In one aspect, MGN modification can be used to increase stability of sgRNA that is used by the Cas9 nuclease to recognize its target sequence. Examples for the potential use of MGN-modified RNAs for gene editing are included by reference to Table 3 in Zhao W, Hou X, Vick O G, Dong Y. RNA delivery biomaterials for the treatment of genetic and rare diseases. Biomaterials. 2019 October; 217:119291. doi: 10.1016/j.biomaterials.2019.119291.

As described and used herein, the term “modified RNA” will be understood to refer to a RNA molecule (e.g., oligonucleotide, or polynucleotide) modified (e.g., covalently attached to) with a MGN as described herein (e.g., pyranosides linked to oligo or polynucleotides by way of (poly)phosphate esters).

In another aspect, the current invention relates to using modification with MGNs to increase stability and efficacy of RNA derivatives that mimic the function of endogenous miRNA (miRNA mimics) as well as molecules that target endogenous miRNA (antimiRs). Due to their biological roles and properties, miRNA mimics and antimiRs can be used in cancer therapies as well as for genetic and rare diseases. For example, a synthetic miRNA mimic of miR-29 designed to reduce the expression of collagen and other scar formation related proteins in the diseases Scleroderma and Keloids.

MGN-modified RNA derivatives can be delivered using nanotechnologies as well as ligand-RNA conjugates, lipid or polymer-based nanoparticles, and cell derived vehicles (exosomes). Many of these biomaterials exhibit organ targeting properties, which make it possible for RNA therapeutics to achieve targeted effects at diseased sites.

In some embodiments, a modified RNA of methods and compositions of the present disclosure is an in vitro-synthesized RNA molecule.

In some embodiments, a modified RNA of methods and compositions of the present disclosure is a therapeutic oligoribonucleotide.

In some embodiments, the present disclosure provides a method for delivering a recombinant protein to a subject, the method comprising the step of contacting the subject with a modified RNA or a gene-therapy vector of the present disclosure, thereby delivering a recombinant protein to a subject.

In some embodiments, the present disclosure provides a double-stranded RNA (dsRNA) molecule comprising a MGN and further comprising an siRNA or short hairpin RNA (shRNA). In another embodiment, the dsRNA molecule is greater than 50 nucleotides in length.

In some embodiments, the length of a modified RNA (e.g. a single-stranded RNA (ssRNA) or dsRNA molecule) of methods and compositions of the present disclosure is greater than 30 nucleotides in length. In some embodiments, a modified RNA is greater than 35, 40, 45, 50, 55, 60, 70, 80, 90 or 100 nucleotides in length.

In some embodiments, the present disclosure provides a method for inducing a mammalian cell to produce a protein of interest, comprising contacting the mammalian cell with an in vitro-synthesized RNA molecule encoding the recombinant protein, the in vitro-synthesized RNA molecule comprising a MGN, thereby inducing a mammalian cell to produce a protein of interest. In some embodiments, the protein of interest is a recombinant protein.

In some embodiments, the present disclosure provides a method of inducing a mammalian cell to produce a recombinant protein, comprising contacting the mammalian cell with an in vitro-transcribed RNA molecule encoding the recombinant protein, the in vitro-transcribed RNA molecule further comprising a MGN, thereby inducing a mammalian cell to produce a recombinant protein.

In some embodiments, a modified RNA of the present disclosure is translated in the cell more efficiently than an unmodified RNA molecule with the same sequence.

Methods of determining translation efficiency are well known in the art, and include, e.g. measuring the activity of an encoded reporter protein (e.g luciferase or renilla or green fluorescent protein [Wall A A, Phillips A M et al, Effective translation of the second cistron in two Drosophila dicistronic transcripts is determined by the absence of in-frame AUG codons in the first cistron. J Biol Chem 2005; 280 (30): 27670-8]), or measuring radioactive label incorporated into the translated protein (Ngosuwan J, Wang N M et al, Roles of cytosolic Hsp70 and Hsp40 molecular chaperones in post-translational translocation of presecretory proteins into the endoplasmic reticulum. J Biol Chem 2003; 278 (9): 7034-42).

In some embodiments, a modified RNA of methods and compositions of the present invention is significantly less immunogenic than an unmodified in vitro-synthesized RNA molecule with the same sequence.

In some embodiments, the relative immunogenicity of a modified RNA and its unmodified counterpart are determined by determining the quantity of the modified RNA required to elicit one of the above responses to the same degree as a given quantity of the unmodified RNA. For example, if twice as much modified RNA is required to elicit the same response, than the modified RNA is two-fold less immunogenic than the unmodified RNA.

In some embodiments, a method of present disclosure further comprises mixing a modified RNA with a transfection reagent prior to the step of administering or contacting with a cell. In some embodiments, a method of present disclosure further comprises administering a modified RNA together with the transfection reagent. In some embodiments, a transfection reagent is a cationic lipid reagent

In some embodiments, the transfection reagent is a lipid-based transfection reagent. In some embodiments, the transfection reagent is a protein-based transfection reagent. In some embodiments, the transfection reagent is a polyethyleneimine based transfection reagent. In some embodiments, the transfection reagent is calcium phosphate. In some embodiments, the transfection reagent is Lipofectin® or Lipofectamine®. In some embodiments, the transfection reagent is any other transfection reagent known in the art.

In some embodiments, the transfection reagent forms a liposome. Liposomes, in some embodiments, increase intracellular stability, increase uptake efficiency and improve biological activity. In some embodiments, liposomes are hollow spherical vesicles composed of lipids arranged in a similar fashion as those lipids which make up the cell membrane. In some embodiments, liposomes can deliver RNA to cells in a biologically active form.

A variety of disorders may be treated by employing methods of the present disclosure including, inter alia, monogenic disorders, infectious diseases, acquired disorders, cancer, and the like.

In some embodiments, the present disclosure provides a method of reducing an immunogenicity of a RNA molecule, the method comprising the step of replacing a nucleotide of the RNA molecule with a modified nucleotide that is or comprises a MGN, thereby reducing immunogenicity of the RNA molecule.

In some embodiments, the present disclosure provides a method of reducing an immunogenicity of a gene-therapy vector comprising a RNA molecule, the method comprising the step of replacing a nucleotide of the RNA molecule with a modified nucleotide that is or comprises a MGN, thereby reducing an immunogenicity of a gene-therapy vector.

In some embodiments, the present disclosure provides a method of enhancing in vitro translation from a RNA molecule, the method comprising the step of replacing a nucleotide of the RNA molecule with a modified nucleotide that is or comprises a MGN, thereby enhancing in vitro translation from the RNA molecule.

In some embodiments, the present disclosure provides a method of enhancing in vivo translation from a gene-therapy vector comprising a RNA molecule, the method comprising the step of replacing a nucleotide of the RNA molecule with a modified nucleotide that is or comprises a MGN, thereby enhancing in vivo translation from a gene-therapy vector.

In some embodiments, the present disclosure provides a method of increasing efficiency of delivery of a recombinant protein by a gene therapy vector comprising a RNA molecule, the method comprising the step of replacing a nucleotide of the RNA molecule with a modified nucleotide that is or comprises a MGN, thereby increasing efficiency of delivery of a recombinant protein by a gene therapy vector.

In some embodiments, the present disclosure provides a method of increasing in vivo stability of gene therapy vector comprising RNA molecule, the method comprising the step of replacing a nucleotide of the RNA molecule with a modified nucleotide that is or comprises a MGN, thereby increasing in vivo stability of gene therapy vector.

In another aspect, the present invention comprises methods of making therapeutic compositions comprising formulating an effective amount of one or more purified or synthetically-produced MGN (or a pharmaceutically-acceptable salt, prodrug or derivative thereof) into a pharmaceutical composition selected from the group consisting of: injectible liquid, tablet, capsule, pill, solution or suspension for oral administration, solid for suspension or dissolution into a drinkable or injectible liquid, dermal patch, eye drop, cream, ointment, gel, powder, spray, and inhalable.

In another aspect, the present invention provides pharmaceutical compositions containing MGNs. In certain embodiments, the invention encompasses a pharmaceutical composition or a single unit dosage form of any of the compounds or therapeutic compositions described above. In certain embodiments, pharmaceutical compositions and single unit dosage forms of the invention comprise a prophylactically or therapeutically effective amount of one or more of the modified RNA molecules describe above, and typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Examples of suitable pharmaceutical carriers are described in “Remington's Pharmaceutical Sciences” by E. W. Martin.

Whether a particular excipient is suitable for incorporation into a pharmaceutical composition or dosage form depends on a variety of factors well known in the art including, but not limited to, the way in which the dosage form will be administered to a patient and the specific active ingredients in the dosage form.

A pharmaceutical composition of the invention is formulated to be compatible with its intended route of administration.

### III. Chemical Compositions of Matter

In another aspect, the present invention encompasses novel compositions of matter including compositions of novel molecules. While some of the MGNs and modified RNAs are naturally occurring molecules that have been detected in the bodies of nematodes, pure samples of these molecules and in particular bulk samples of the pure MGNs and modified RNAs free from other biological materials are not found in nature. Additionally, many of the MGNs and modified RNAs described above have not been detected in nature, even with the aid of highly sensitive and selective analytical techniques such as HPLC-coupled high resolution mass spectroscopy. As such, many of the compounds described above constitute novel compositions of matter.

In certain embodiments, the present invention provides pure sample of any of the MGNs and modified RNAs described above and in the genera and subgenera herein. In certain embodiments, the present invention provides samples comprising bulk quantities of such molecules in substantially pure form. In certain embodiments, the present invention provides novel compositions comprising mixtures of between two and ten different MGNs or modified RNAs.

### IV. Methods of Formulation

In another aspect, the present invention comprises methods of making therapeutic compositions comprising formulating an effective amount of one or more purified or synthetically-produced MGNs (or a pharmaceutically-acceptable salt, prodrug or derivative thereof) into a therapeutic composition. In certain embodiments, such therapeutic compositions are selected from the group consisting of: an injectible liquid, a tablet, a capsule, a pill, a solution or suspension for oral administration, a solid dosage form for suspension or dissolution into a drinkable- or injectible liquid, a dermal patch, an eye drop, a cream, an ointment, a gel, a powder, a spray, an inhalable composition, and a nasal spray. In some embodiments, a thereapeutic composition includes a lipid nanoparticle comprising a modified RNA described herein.

### EXAMPLES

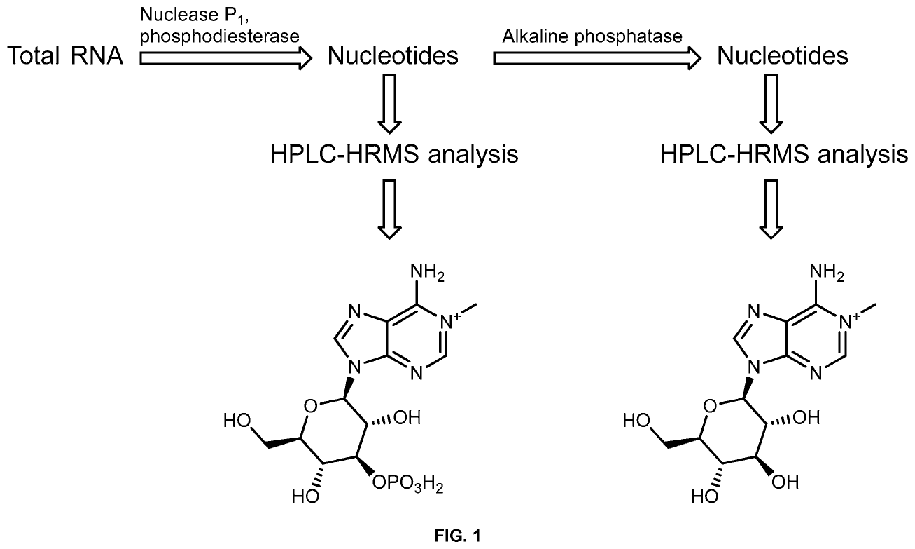

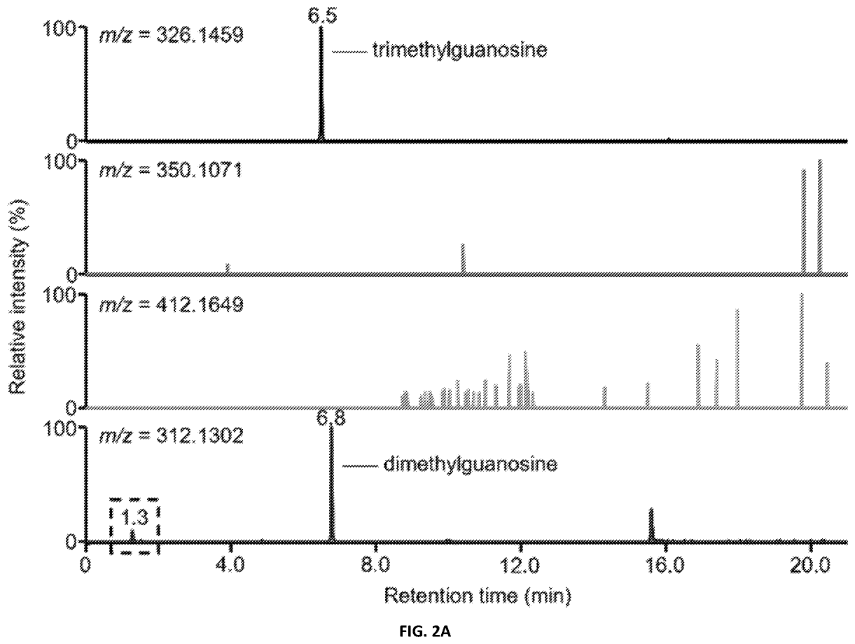

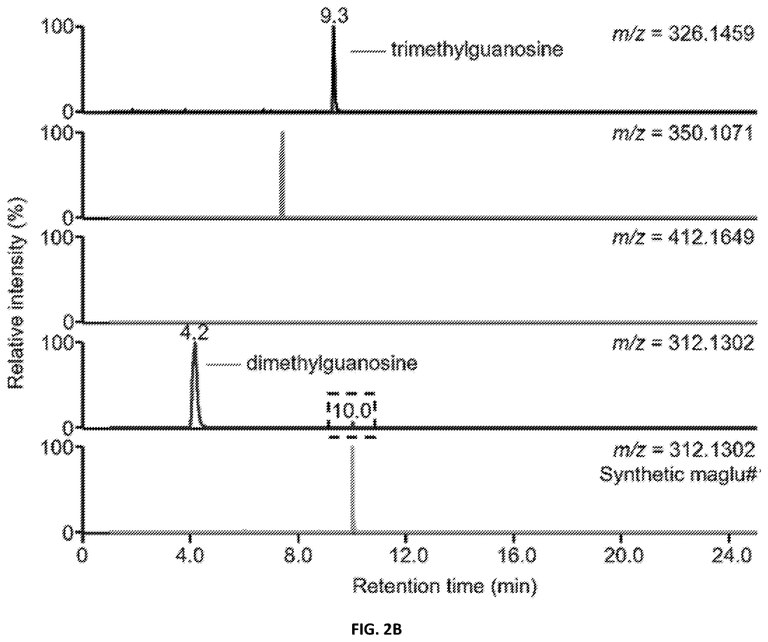

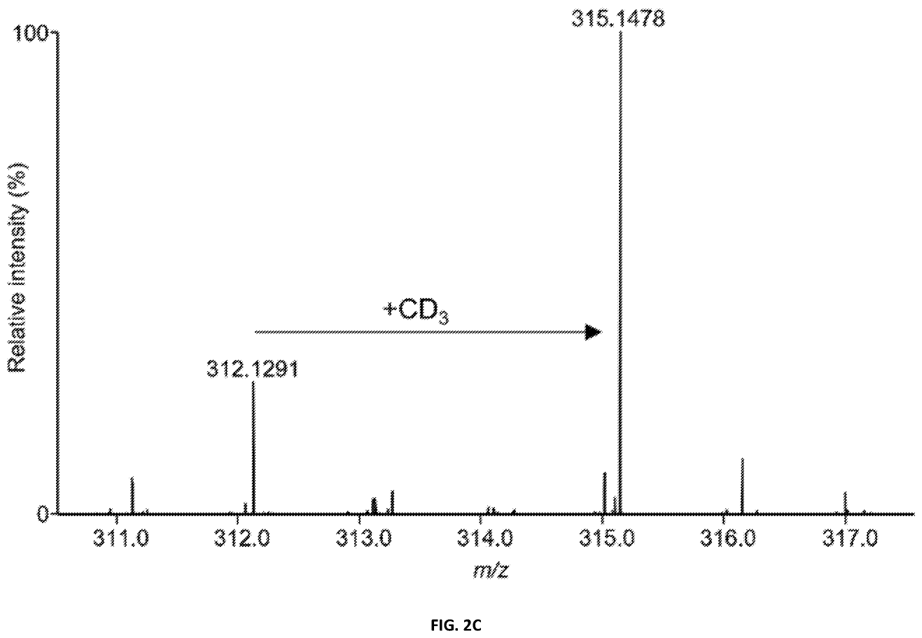

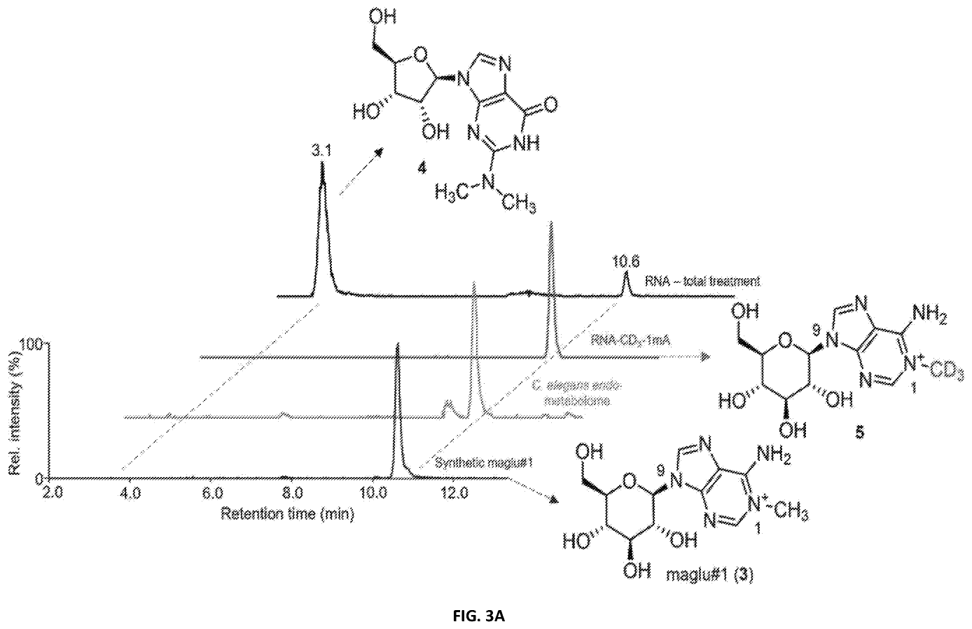

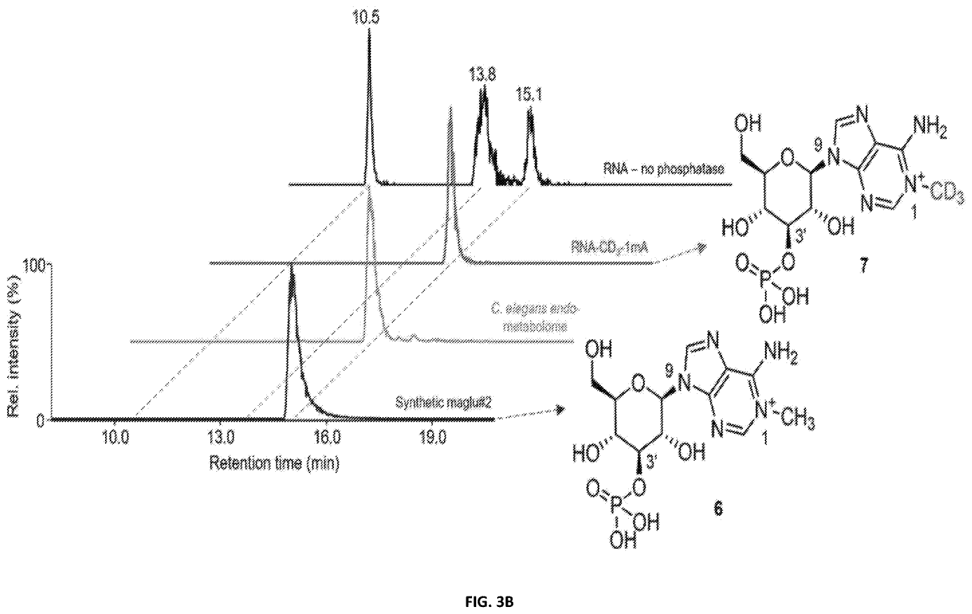

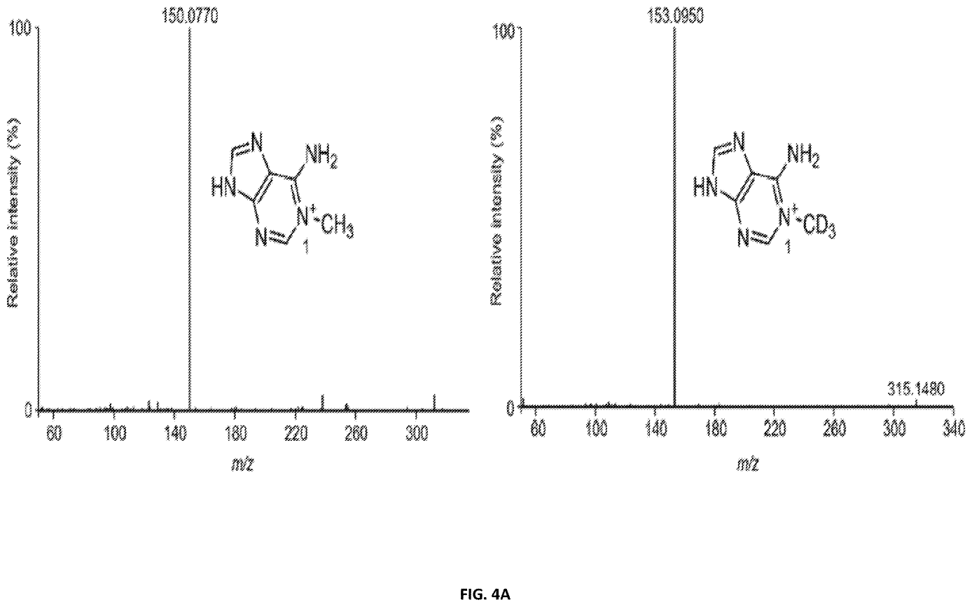

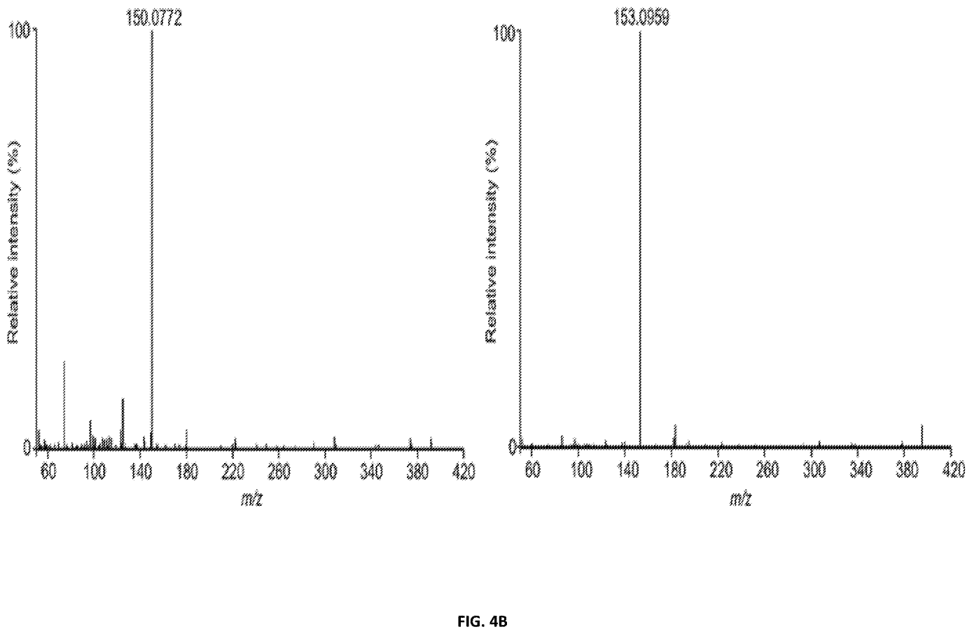

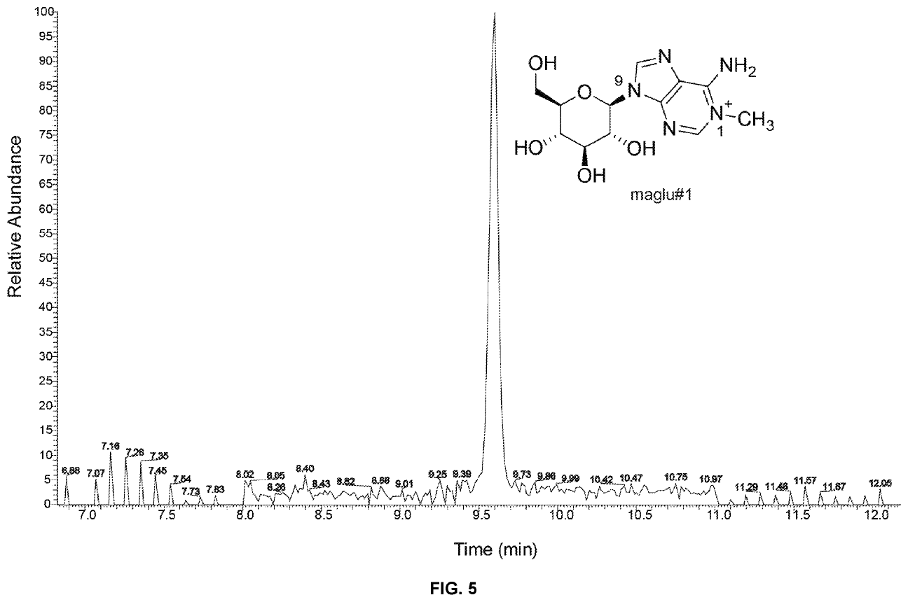

The following Examples are useful to confirm aspects of the disclosure described above and to exemplify certain embodiments of the disclosure. It will be understood by those skilled in the art that the formation of a phosphate ester and polyphosphate esters is well documented in the literature, e.g., RNA 2012 April 18 (4): 856-868; Beilstein J. Org. Chem. 2017, 13, 2819-2832. Those skilled in the art will recognize that phosphate esters and equivalents thereof as described herein can be readily joined through covalency by a variety of formal condensation transformations to form newly substituted phosphoryl derivatives, for example diphosphate esters. Those skilled in the art will also recognize that phosphates such as gluconucleoside phosphates of the instant invention can be attached to RNA and DNA by multiple means including, without limitation, solid phase synthesis (see, e.g., Flamme, et al. (2019). Methods 161:64-82 (e.g., FIG. 1A); Tang, et al. (2017) ACS Omega, 2 (11): 8205-8212; Mikhailov, et al. (2002) Nucleic Acids Res., 30 (5): 1124-1131 (e.g., Scheme 1)).

### Methods for Detecting Gluconucleosides in RNA of Nematodes

RNA extraction and hydrolysis. Total RNA was isolated from C. elegans and C. briggsae using mirVana RNA isolation kit (mirVana™ miRNA Isolation Kit, with phenol, catalog number: AM1560). RNA was isolated following manufacturer's instruction. Regarding stable isotope labeled samples, nematodes were treated with CD3-N¹-methyladenine (200 μM) for 48 hr and RNA isolated following manufacturer's instructions.

Isolated RNAs were hydrolyzed as previously described (Thuring, K.; Schmid, K.; Keller, P.; Helm, M. LC-MS Analysis of Methylated RNA. Methods Mol. Biol. 2017, 1562, 3-18). In summary, 10 μg of RNA was first treated with nuclease P1 (Fisher Scientific) and Phosphodiesterase I from Crotalus adamanteus venom (Sigma) and incubated at 37° C. for 2 hr. Next, RNA was treated with alkaline phosphatase (Thermo) and incubated to 37° C. for 1 hr. Upon completion, samples were kept on ice until diluted 1/10 (v/v) in HPLC vials using MQ H₂O and stored at −20° C. prior until analysis via HPLC-MS. In addition, the enzymatic treatment process was also performed without use of alkaline phosphatase.

HPLC-MS analysis. Several methods for chromatographic separation were utilized due to varying polarity of nucleosides. High resolution LC-MS analysis was performed on a Thermo Fisher Scientific Vanquish Horizon UHPLC System coupled with a Thermo Q Exactive HF hybrid quadrupole-orbitrap high resolution mass spectrometer equipped with a HESI ion source. 2 μL of RNA extract samples and 1 μL of synthetic and endo-metabolome samples (C. elegans N2) were analyzed using several different methods, as specified below:

Method A′—water-acetonitrile gradient on a Zorbax HILIC Plus column (150 mm×2.1 mm 1.8 μm particle size 95 Å pore size, Agilent) and maintained at 40° C. Solvent A: 0.1% formic acid in water; solvent B: 0.1% formic acid in acetonitrile. A/B gradient started at 95% B for 4 min, then from 95% to 55% B over 15 min, then rapidly down to 5% B and held for 3 min, then back to 95% B and equilibrated for 3 min.

Method B′—water-acetonitrile gradient on a XBridge Amide column (150 mm×2.1 mm 3.5 μm particle size 130 Å pore size, Waters) and maintained at 40° C. Solvent A: 90% acetonitrile and 10% water prepared with 0.4% (v/v) of 25% ammonia in water solution combined with 0.1% (v/v) formic acid, solvent B: 30% acetonitrile and 70% water prepared with 0.4% (v/v) of 25% ammonia in water solution combined with 0.1% (v/v) formic acid. A/B gradient started at 1% B for 3 min, then from 1% to 60% B over 17 min, then from 60% to 100% B over 6 min and held for 1.5 min, then back to 1% B over 0.5 min and equilibrated for 2 min.

Method C′—water-methanol gradient on a ACQUITY UPLC BEH C18 Column (50 mm×2.1 mm 1.7 μm particle size 130 Å pore size, Waters) and maintained at 40° C. Solvent A: 0.1% ammonium formate in water; solvent B: methanol. A/B gradient started at 0% B for 3 min, then from 0% to 100% B over 17 min, held at 100% B for 2 min, then rapidly down to 0% B over 1 min, and held for 2 min to equilibrate the column. Mass spectrometer parameters: 3.5 kV spray voltage, 380° C. capillary temperature, 300° C. probe heater temperature, 60 sheath flow rate, 20 auxiliary flow 15 rate, 1 spare gas; S-lens RF level 50.0, resolution 240,000, m/z range 100-1200, AGC target 3e6. The instrument was calibrated with positive and negative ion calibration solutions (Thermo-Fisher) Pierce LTQ Velos ESI pos/neg calibration solutions. Peak areas were determined using Xcalibur 2.3 QualBrowser version 2.3.26 (Thermo Scientific) using a 5-10 ppm window around the m/z of interest.

### A. Synthetic Procedures

General synthetic procedures. Unless noted otherwise, all chemicals and reagents were purchased from Sigma-Aldrich. All oxygen and moisture-sensitive reactions were carried out under argon atmosphere in flame-dried glassware. Solutions and solvents sensitive to moisture and oxygen were transferred via standard syringe and cannula techniques. All commercial reagents were purchased as reagent grade and, unless otherwise stated, were purchased from Sigma-Aldrich and used without any further purification. Boc-2-Abz-OH was purchased from Chem-impex. Acetic acid (AcOH), acetonitrile (ACN), dichloromethane (DCM), ethyl acetate (EtOAc), formic acid, hexanes and methanol (MeOH) used for chromatography and as a reagent or solvent were purchased from Fisher Scientific. Thin-layer chromatography (TLC) was performed using J. T. Baker Silica Gel IB2F plates. Flash chromatography was performed using Teledyne Isco CombiFlash systems and Teledyne Isco RediSep Rf silica and C18 columns. All 20 deuterated solvents were purchased from Cambridge Isotopes. Nuclear Magnetic Resonance (NMR) spectra were recorded on Bruker INOVA 500 (500 MHz) and Varian INOVA 600 (600 MHz) spectrometers at Cornell University's NMR facility and Bruker AVANCE III HD 800 MHz (800 MHz) or Bruker AVANCE III HD 600 MHz (600 MHz) at SUNY ESF's NMR facility. ¹H NMR chemical shifts are reported in ppm (δ) relative to 25 residual solvent peaks (7.26 ppm for chloroform-d, 3.31 ppm for methanol-d₄, 2.50 for DMSO-d₆). NMR-spectroscopic data are reported as follows: chemical shift, multiplicity (s=singlet, d=doublet, t=triplet, q=quartet, m=multiplet, br=broad), coupling constants (Hz), and integration and often tabulated including 2D NMR data. ¹³C NMR chemical shifts are reported in ppm (δ) relative to residual solvent peaks (77.16 ppm for 30 chloroform-d, 49.00 ppm for methanol-d₄, 39.52 for DMSO-d₆). All NMR data processing was done using MNOVA 14.2.1 (mestrelab.com).

### Abbreviations

HPLC-HRMS, high performance liquid chromatography-high resolution mass spectrometry; MS/MS, tandem mass spectrometry; LRO, lysosome related organelle; UGT, uridine diphosphoglucuronosyltransferase: UDP, uridine 5′-diphosphate; CEST, carboxylesterase; ESI−, electrospray ionization negative mode; ESI+, electrospray ionization positive mode; mCPBA, 3-chloroperoxy benzoic acid.

iglu #1 (4) was synthesized as described previously. (Eur. J. Med. Chem. 2004, 39 (5), 453-458. doi.org/10.1016/j.ejmech.2004.01.001.)

iglu #3 (10) was synthesized as described previously. (Elife 2020, 9, 1-42. doi.org/10.7554/eLife.61886)

Iglu #301 (31) was synthesized as described previously. (Elife 2020, 9, 1-42. doi.org/10.7554/eLife.61886)

### Reagents and General Procedures

All oxygen and moisture-sensitive reactions were carried out under argon atmosphere in flame-dried glassware. Solutions and solvents sensitive to moisture and oxygen were transferred via standard syringe and cannula techniques. Trimethylsilyl trifluoromethanesulfonate (TMSOTf) was transferred to a Schlenk flask prior to use and stored at −20° C. Methanolic ammonia (7N) was purchased from Acros Organics. All commercial reagents were purchased as reagent grade and, unless otherwise stated, were purchased from Sigma-Aldrich and used without any further purification. Acetic acid (AcOH), acetonitrile (ACN), dichloromethane (DCM), ethylacetate (EtOAc), N,N-dimethylformamide (DMF), tetrahydrofuran (THF), dimethylsulfoxide (DMSO), formic acid, hexanes, and methanol (MeOH) used for chromatography and as a reagent or solvent were purchased from ThermoFisher Scientific. Acetyl chloride (1-¹³C, 99%) was purchased from Cambridge Isotope Laboratories, N-acetylserotonin (NAS) was obtained from Biosynth International, Boc-2-aminobenzoic acid (Boc-2-Abz-OH) was from Chem-Impex International, and trifluoroacetic acid (TFA) was from Tokyo Chemical Industry, fluoxetine hydrochloride was from Spectrum Chemical. Dichloromethane (DCM), and N,N-dimethylformamide (DMF) were dried with 3 Å molecular sieves prior to use. Thin-layer chromatography (TLC) was performed using J. T. Baker Silica Gel IB2F plates. Flash chromatography was performed using Teledyne IscoCombiFlash systems and Teledyne Isco RediSep Rf silica and C18 reverse phase columns. All deuterated solvents were purchased from Cambridge Isotopes. Abbreviations used: triethylamine (TEA), 2,3-dichloro-5,6-dicyano-1,4-benzoquinone (DDQ), trichloroacetonitrile (CCl₃CN), 1,8-diazabicyclo[5.4.0]undec-7-ene (DBU), trifluoromethanesulfonate (TMSOTf), N-ethyl-N′-(3-dimethylaminopropyl) carbodiimide hydrochloride (EDC·HCl), 4-dimethylaminopyridine (DMAP), 1,3-dichloro-1,1,3,3-tetraisopropyldisiloxane (TIPDSiCl₂), 3-chloroperoxybenzoic acid (m-CPBA). Nuclear Magnetic Resonance (NMR) spectra were recorded on Bruker INOVA 500 (500 MHz) and Varian INOVA 600 (600 MHz) spectrometers at Cornell University's NMR facility and Bruker AVANCE III HD 800 MHz (800 MHz) or Bruker AVANCE III HD 600 MHz (600 MHz) at SUNY ESF's NMR facility. ¹H NMR chemical shifts are reported in ppm (δ) relative to residual solvent peaks (7.26 ppm for chloroform-d, 3.31 ppm for methanol-d₄, 2.05 ppm for acetone-d₆). NMR-spectroscopic data are reported as follows: chemical shift, multiplicity (s=singlet, d=doublet, t=triplet, q=quartet, m=multiplet, br=broad), coupling constants (Hz), and integration and often tabulated including 2D NMR data.¹³CNMR chemical shifts are reported in ppm (δ) relative to residual solvent peaks (77.16 ppm for chloroform-d, 49.00 ppm for methanol-d₄, 29.9 ppm for acetone-d₆). All NMR data processing was done using MestreLab MNOVA version 14.2.1-27684 (mestrelab.com).

### High-Performance Liquid Chromatography-Mass Spectrometry (HPLC-MS) for Characterization of Synthetic Compounds

Several methods for chromatographic separation were utilized due to varying polarity of metabolites of interest. High resolution LC-MS analysis was performed on a Thermo Fisher Scientific Vanquish Horizon UHPLC System coupled with a Thermo Q Exactive HF hybrid quadropole-orbitrap high resolution mass spectrometer quipped with a HESI ion source. 1 μL of synthetic and natural endo- and exo-metabolome extracts (C. elegans N2, C. briggsae AF-16, C. elegans him-5, and C. elegans fem-3 (gf)) were injected and separated according to the methods provided below:

Method A—water-acetonitrile gradient on a Hypersil GOLD C18 column (150 mm×2.1 mm 1.9 um particle size 175 Å pore size, Thermo Scientific) and maintained at 40° C. Solvent A: 0.1% formic acid in water; solvent B: 0.1% formic acid in acetonitrile. A/B gradient started at 1% B for 3 min, then from 1% to 99% B over 17 min, 99% B for 5 min, then rapidly down to 1% B over 0.5 min and held for 2.5 min to equilibrate the column.

Method B—water-acetonitrile gradient on a Hypersil GOLD C18 column (150 mm×2.1 mm 1.9 um particle size 175 Å pore size, Thermo Scientific) and maintained at 40° C. Solvent A: 0.1% formic acid in water; solvent B: 0.1% formic acid in acetonitrile. A/B gradient started at 1% B for 3 min, then from 1% to 35% B over 37 min, then from 35% to 100% B over 15 min, held at 100% B for 2 min, then rapidly down to 1% B over 0.5 min, and held for 2.5 min to equilibrate the column.

Method C—water-acetonitrile gradient on a Zorbax HILIC Plus column (150 mm×2.1 mm 1.8 um particle size 95 Å pore size, Agilent) and maintained at 40° C. Solvent A: 0.1% formic acid in water; solvent B: 0.1% formic acid in acetonitrile. A/B gradient started at 95% B for 4 min, then from 95% to 55% B over 15 min, then rapidly down to 5% B and held for 3 min, then back to 95% B and equilibrated for 3 min.

Method D—water-acetonitrile gradient on a XBridge Amide column (150 mm×2.1 mm 3.5 um particle size 130 Å pore size, Waters) and maintained at 40° C. Solvent A: 90% acetonitrile and 10% water prepared with 0.4% (v/v) of 25% ammonia in water solution combined with 0.1% (v/v) formic acid, solvent B: 30% acetonitrile and 70% water prepared with 0.4% (v/v) of 25% ammonia in water solution combined with 0.1% (v/v) formic acid. A/B gradient started at 1% B for 3 min, then from 1% to 60% B over 17 min, then from 60% to 100% B over 6 min and held for 1.5 min, then back to 1% B over 0.5 min and equilibrated for 2 min.

Method E—water-acetonitrile gradient on a XBridge Amide column (150 mm×2.1 mm 3.5 um particle size 130 Å pore size, Waters) and maintained at 40° C. Solvent A: 90% acetonitrile and 10% water prepared with 0.4% (v/v) of 25% ammonia in water solution combined with 0.1% (v/v) formic acid, solvent B: 30% acetonitrile and 70% water prepared with 0.4% (v/v) of 25% ammonia in water solution combined with 0.1% (v/v) formic acid. A/B gradient started at 1% B for 3 min, then from 1% to 35% B over 37 min, then from 35% to 100% B over 15 min and held for 2 min, then back to 1% B over 0.5 min and equilibrated for 2.5 min.

Mass spectrometer parameters: 3.5 kV spray voltage, 380° C. capillary temperature, 300° C. probe heater temperature, 60 sheath flow rate, 20 auxiliary flow 15 rate, 1 spare gas; S-lens RF level 50.0, resolution 240,000, m/z range 100-1200 m/z, AGC target 3e6. Instrument was calibrated with positive and negative ion calibration solutions (Thermo-Fisher) Pierce LTQ Velos ESI pos/neg calibration solutions. Peak areas were determined using Xcalibur 2.3 QualBrowser version 2.3.26 (Thermo Scientific) using a 5-10 ppm window around the m/z of interest.

### Example 1. Step 1

### (6aR,8R,9R,10R,10aS)-8-(1H-indol-1-yl)-2,2,4,4-tetraisopropylhexahydropyrano[3,2-f][1,3,5,2,4]trioxadisilocine-9,10-diol (S1)

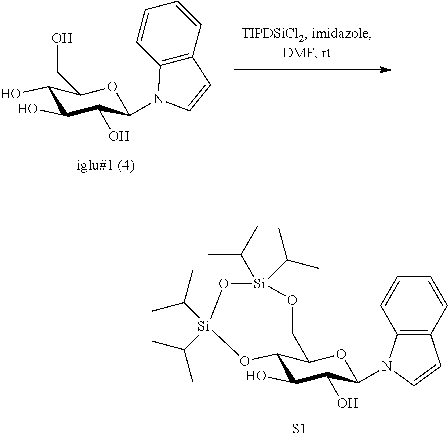

To 2.5 mL of DMF was added iglu #1 (4, 144.6 mg, 0.518 mmol, 1.0 equiv.) and imidazole (155.0 mg, 2.28 mmol, 4.4 equiv.). The stirred mixture was cooled to 0° C. before adding 1,3-dichloro-1,1,3,3-tetraisopropyldisiloxane (215 μL, 0.673 mmol, 1.3 equiv.). The reaction mixture was stirred at room temperature for 30 min, diluted with DCM, and then quenched with water. The organics were washed with sat. aq. NaHCO₃, dried with Na₂SO₄, and concentrated in vacuo. Flash column chromatography on silica using a gradient of 0-20% MeOH in DCM afforded S1 (250.5 mg, 93%) as an orange oil. ¹H NMR (500 MHz, chloroform-d): δ (ppm) 7.60 (d, J=7.8 Hz, 1H), 7.50 (d, J=8.2 Hz, 1H), 7.24 (d, J=3.5 Hz, 1H), 7.19 (ddd, J=1.2, 7.6, 8.6 Hz, 1H), 7.12 (ddd, J=0.9, 7.5, 8.0 Hz, 1H), 5.36 (d, J=8.9 Hz, 1H), 4.13 (dd, J=2.0, 12.7 Hz, 1H), 4.04 (t, J=7.7 Hz, 1H), 4.02 (t, J=8.2 Hz, 1H), 3.97 (dd, J=1.6, 12.7 Hz, 1H), 3.80 (t, J=9.0 Hz, 1H), 3.47 (dt, J=1.6, 9.0 Hz, 1H), 1.17-1.03 (m, 28H).

### Example 1. Step 2

### (6aR,8R,9R,10R,10aS)-10-hydroxy-8-(1H-indol-1-yl)-2,2,4,4-tetraisopropyl-hexahydropyrano[3,2-f][1,3,5,2,4]trioxadisilocin-9-yl benzoate (16)

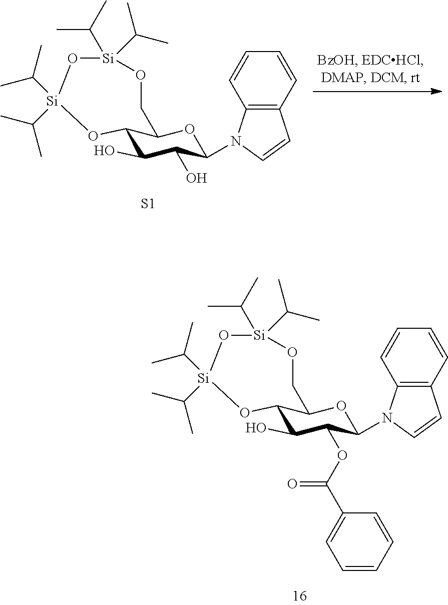

To a stirred solution of benzoic acid (14.4 mg, 0.118 mmol, 1.0 equiv.) in DCM, EDC·HCl (45.2 mg, 0.236 mmol, 2.0 equiv.) was added. The mixture was stirred at room temperature for 40 min, and S1 (73.8 mg, 0.142 mmol, 1.2 equiv.) and DMAP (36.0 mg, 0.295 mmol, 2.5 equiv.) were added. The reaction mixture was stirred at room temperature for 2.5 hours. The reaction mixture was concentrated in vacuo followed by flash column chromatography on silica using a gradient of 5-80% EtOAc in hexanes affording 16 (72.3 mg, 98%). ¹H NMR (500 MHz, chloroform-d): δ (ppm) 7.77 (dd, J=1.2, 8.5 Hz, 2H), 7.52 (d, J=7.8 Hz, 1H), 7.49-7.44 (m, 2H), 7.32-7.28 (m, 3H), 7.18 (ddd, J=1.0, 7.7, 8.3 Hz, 1H), 7.07 (ddd, J=0.8, 7.6, 8.3 Hz, 1H), 6.46 (d, J=3.4 Hz, 1H), 5.72 (d, J=9.2 Hz, 1H), 5.66 (t, J=9.0 Hz, 1H), 4.17 (dd, J=2.1, 12.6 Hz, 1H), 4.14 (t, J=9.0 Hz, 1H), 4.08 (t, J=9.0 Hz, 1H), 4.02 (dd, J=1.2, 12.6 Hz, 1H), 3.55 (dt, J=1.6, 9.2 Hz, 1H), 1.19-1.03 (m, 28H).

### Example 1. Step 3

### (6aR,8R,9R,10R,10aR)-10-((bis(benzyloxy)phosphoryl)oxy)-8-(1H-indol-1-yl)-2,2,4,4-tetraisopropylhexahydropyrano[3,2-f][1,3,5,2,4]trioxadisilocin-9-yl benzoate (19)

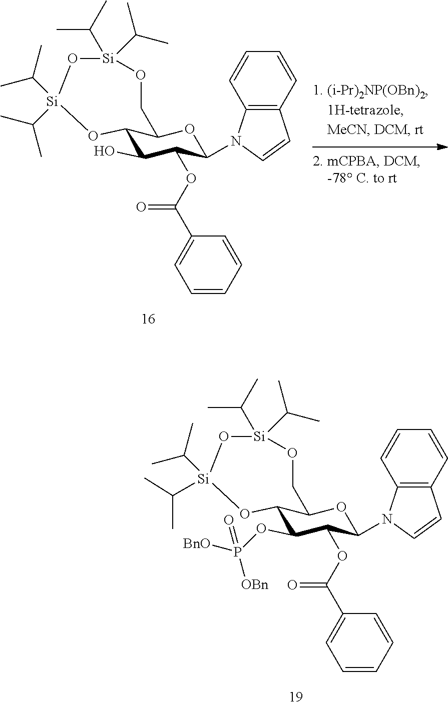

To a solution of 16 (32.5 mg, 0.052 mmol, 1.0 equiv.) in 0.8 mL DCM was added dibenzyl N,N-diisopropylphosphoramidite (105 μL, 0.312 mmol, 6.0 equiv.) and 1H-tetrazole (0.45 M in ACN, 693 μL, 0.312 mmol, 6.0 equiv.). The reaction mixture was stirred at room temperature for 30 min. Then the solution was cooled to −78° C. under argon before adding 3-chloroperoxybenzoic acid (mCPBA, ~77%, 81.6 mg, 0.364 mmol, 7.0 equiv.). The solution was stirred at room temperature for 2 hr. The mixture was diluted with DCM and washed with sat. aq. NaHCO₃, dried with Na₂SO₄, and concentrated in vacuo. Flash column chromatography on silica using a gradient of 0-90% EtOAc in hexanes afforded 19 (32.2 mg, 70%) as a roughly 1:1 mixture with excess dibenzyl diisopropylphosphoramidite. ¹H NMR (500 MHz, chloroform-d): δ (ppm) 7.77 (dd, J=1.3, 8.3 Hz, 2H), 7.49 (d, J=7.7 Hz, 1H), 7.47-7.40 (m, 2H), 7.27 (m, 2H), 7.38-7.28 (m, 9H, with impurity), 7.21-7.14 (m, 3H, with impurity), 7.23 (d, J=3.4 Hz, 1H), 7.14 (m, 1H), 7.03 (ddd, J=0.8, 7.4, 8.3 Hz, 1H), 6.92 (m, 2H), 6.44 (d, J=3.4 Hz, 1H), 5.80 (t, J=9.1 Hz, 1H), 5.66 (d, J=9.1 Hz, 1H), 4.92 (d, J=9.1 Hz, 1H), 4.86 (dd, J=6.8, 11.7 Hz, 1H), 4.78-4.69 (m, 2H), 4.51 (dd, J=9.7, 11.8 Hz, 1H), 4.32 (t, J=9.3 Hz, 1H), 4.19 (dd, J=1.9, 12.7 Hz, 1H), 4.03 (dd, J=1.2, 12.7 Hz, 1H), 1.18 (d, J=7.1 Hz, 3H), 1.17 (d, J=7.8 Hz, 3H), 1.10-0.99 (m, 19H), 0.94 (d, J=6.9 Hz, 3H).

### Example 1. Step 4

### (2R,3R,4S,5R,6R)-4-((bis(benzyloxy)phosphoryl)oxy)-5-hydroxy-6-(hydroxymethyl)-2-(1H-indol-1-yl)tetrahydro-2H-pyran-3-yl benzoate (22)

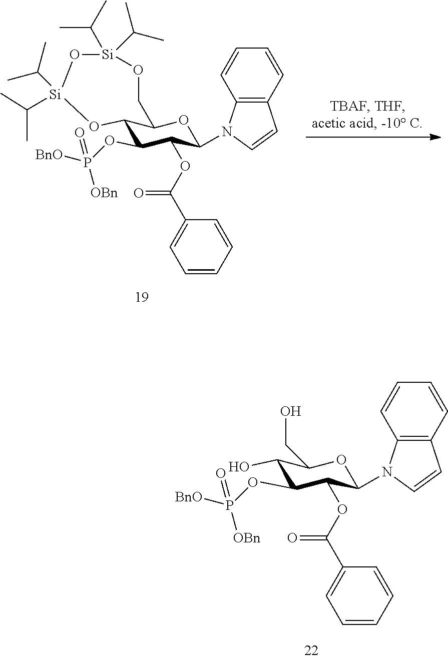

To a solution of 19 (32.2 mg, 0.0364 mmol, 1.0 equiv.) in 1 mL THF was added acetic acid (6 μL, 0.109 mmol, 3.0 equiv.), and the mixture was cooled to −10° C. Tetrabutylammonium fluoride (1M in THF, 109 μL, 0.109 mmol, 3.0 eq) was added, and the solution was stirred for 10 min. Subsequently, acetic acid (15 μL, 0.262 mmol, 7.2 equiv.) was added, and the reaction mixture was concentrated in vacuo. Flash column chromatography on silica using a gradient of 0-40% MeOH in DCM afforded 22 (23.1 mg, 99%). Product contained 45% of impurity dibenzyl diisopropylphosphoramidite. ¹H NMR (500 MHz, methanol-d₄): δ (ppm) 7.70 (dd, J=1.2, 8.3 Hz, 2H), 7.60 (d, J=8.4 Hz, 1H), 7.43 (m, 1H), 7.41 (br, 1H), 7.24 (m, 2H), 7.10 (m, 1H), 6.97 (m, 1H), 6.94 (m, 2H), 6.40 (d, J=3.3 Hz, 1H), 6.03 (d, J=9.2 Hz, 1H), 6.44 (t, J=9.2 Hz, 1H), 5.07-4.92 (m, 5H), 4.00-3.95 (m, 2H), 3.88-3.90 (m, 2H).

### Example 1. Step 5

### (2R,3R,4S,5R,6R)-5-hydroxy-6-(hydroxymethyl)-2-(1H-indol-1-yl)-4-(phosphonooxy)tetrahydro-2H-pyran-3-yl benzoate (iglu #121, 25)

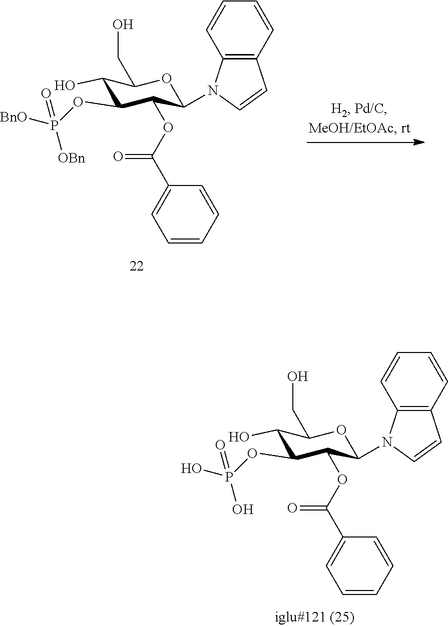

To a mixture of 1:1 MeOH/EtO Ac (v/v, 2 mL) and 22 (23.1 mg, 0.0359 mmol, 1.0 equiv.) was added Pd/C (10% w/w) (20 mg). The reaction mixture was purged with argon for 2 min, then H₂ gas was bubbled through for 45 min at room temperature, and the reaction vessel was again purged with argon for 2 min. The reaction mixture was filtered through Celite and concentrated in vacuo. The crude mixture was purified by reversed-phase flash chromatography with a C18 column using a gradient of 0-60% ACN in H₂O (with 0.1% formic acid), which afforded iglu #121 (25, 2.4 mg, 14%) as clear oil. See Table S1 for NMR spectroscopic data of iglu #121 (25).

### Example 2. Step 1

### (6aR,8R,9R,10R,10aS)-10-hydroxy-8-(1H-indol-1-yl)-2,2,4,4-tetraisopropyl-hexahydropyrano[3,2-f][1,3,5,2,4]trioxadisilocin-9-yl 1H-pyrrole-2-carboxylate (17)

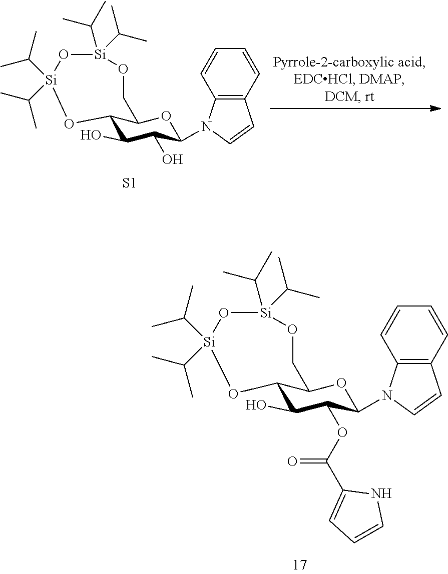

To a stirred solution of pyrrole-2-carboxylic acid (13.4 mg, 0.121 mmol, 1.0 equiv.) in DCM, EDC·HCl (46.0 mg, 0.240 mmol, 2.0 equiv.) was added. The mixture was stirred at room temperature for 30 min, and S1 (75.2 mg, 0.144 mmol, 1.2 equiv.) and DMAP (36.7 mg, 0.30 mmol, 2.5 equiv.) were added. The reaction mixture was stirred at room temperature for 4 hours. The reaction mixture was concentrated in vacuo followed by flash column chromatography on silica using a gradient of 0-80% EtOAc in hexanes, affording 17 (38.7 mg, 44%). ¹H NMR (500 MHz, chloroform-d): δ (ppm) 8.92 (s, 1H), 7.52 (d, J=7.8 Hz, 1H), 7.47 (d, J=8.3 Hz, 1H), 7.26 (d, J=3.4 Hz, 1H), 7.19 (ddd, J=1.0, 7.2, 9.3 Hz, 1H), 7.08 (ddd, J=0.8, 7.0, 8.6 Hz, 1H), 6.76 (m, 1H), 6.72 (m, 1H), 6.45 (d, J=3.4 Hz, 1H), 6.11 (m, 1H), 5.65 (d, J=9.2 Hz, 1H), 5.49 (t, J=9.2 Hz, 1H), 4.15 (dd, J=2.0, 12.7 Hz, 1H), 4.09 (dd, J=3.4, 8.7 Hz, 1H), 4.03-3.98 (m, 2H), 3.52 (dt, J=1.6, 9.2 Hz, 1H), 1.17-1.01 (m, 28H).

### Example 2. Step 2

### (6aR,8R,9R,10R,10aR)-10-((bis(benzyloxy)phosphoryl)oxy)-8-(1H-indol-1-yl)-2,2,4,4-tetraisopropylhexahydropyrano[3,2-f][1,3,5,2,4]trioxadisilocin-9-yl 1H-pyrrole-2-carboxylate (20)

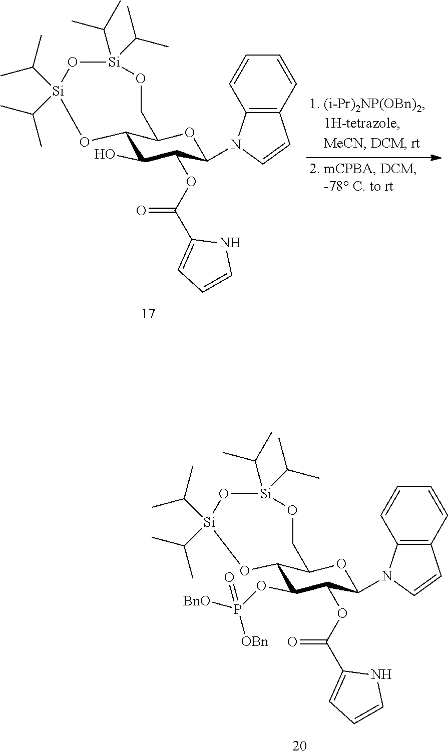

To a solution of 17 (38.7 mg, 0.063 mmol, 1.0 equiv.) in 1 mL DCM was added dibenzyl N,N-diisopropylphosphoramidite (64 μL, 0.189 mmol, 3.0 equiv.) and 1H-tetrazole (0.45 M in ACN, 420 μL, 0.189 mmol, 3.0 equiv.). The reaction mixture was stirred at room temperature for 30 min. Then the solution was cooled to −78° C. under argon before added 3-chloroperoxybenzoic acid (mCPBA, ~77%, 44.0 mg, 0.196 mmol, 3.1 equiv.). The solution was stirred to up room temperature over a 2-hr period. The mixture was diluted with DCM and washed with sat. aq. NaHCO₃, dried with Na₂SO₄, and concentrated in vacuo. Flash column chromatography on silica using a gradient of 0-80% EtOAc in hexanes afforded 20 (47.8 mg, 87%). ¹H NMR (500 MHz, chloroform-d): δ (ppm) 9.68 (s, 1H), 7.53 (dt, J=1.0, 7.7 Hz, 1H), 7.45 (d, J=8.3 Hz, 1H), 7.37-7.32 (m, 3H), 7.27 (d, J=3.4 Hz, 1H), 7.24-7.20 (m, 4H), 7.13 (ddd, J=1.1, 6.9, 7.9 Hz, 1H), 7.02 (ddd, J=0.9, 6.9, 7.9 Hz, 1H), 6.92 (dd, J=1.4, 7.7 Hz, 1H), 6.78 (m, 1H), 6.48 (d, J=3.4 Hz, 1H), 6.11 (m, 1H), 5.63 (t, J=9.0 Hz, 1H), 5.58 (d, J=8.9 Hz, 1H), 5.02 (m, 1H), 4.97 (dd, J=4.8, 12.0 Hz, 1H), 4.95-4.88 (m, 2H), 4.68 (dd, J=7.2, 11.7 Hz, 1H), 4.53 (dd, J=8.4, 11.7 Hz, 1H), 4.29 (t, J=9.4 Hz, 1H), 4.17 (dd, J=1.9, 12.7 Hz, 1H), 4.01 (dd, J=1.2, 12.7 Hz, 1H), 3.50 (m, 1H), 1.24 (d, J=6.7 Hz, 3H), 1.18 (d, J=6.9 Hz, 3H), 1.11-0.93 (m, 22H).

### Example 2. Step 3

### (2R,3R,4S,5R,6R)-4-((bis(benzyloxy)phosphoryl)oxy)-5-hydroxy-6-(hydroxymethyl)-2-(1H-indol-1-yl)tetrahydro-2H-pyran-3-yl 1H-pyrrole-2-carboxylate (23)

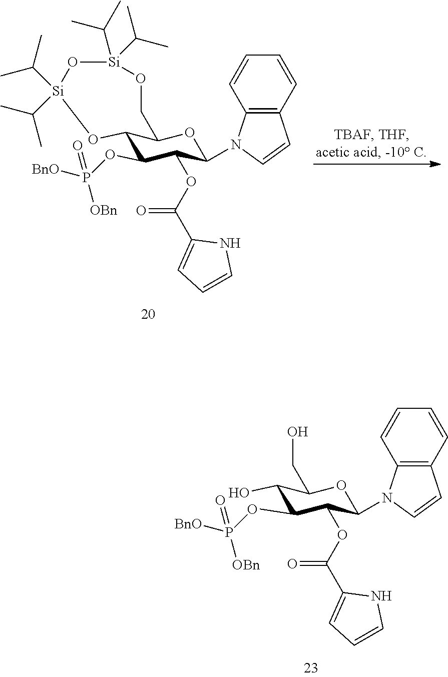

To a solution of 20 (47.8 mg, 0.0547 mmol, 1.0 equiv.) in 1 mL THF was added acetic acid (9.4 μL, 0.164 mmol, 3.0 equiv.) and cooled to −10° C. The solution was added tetrabutylammonium fluoride (1M in THF, 164 μL, 0.164 mmol, 3.0 equiv.) and stirred for 1.5 hr. The reaction mixture was added acetic acid (10 μL, 0.175 mmol, 3.2 equiv.) and concentrated in vacuo. Flash column chromatography on silica using a gradient of 0-40% MeOH in DCM afforded 23 (28.4 mg, 82%). ¹H NMR (500 MHz, methanol-d₄): δ (ppm) 7.59 (d, J=8.4 Hz, 1H), 7.44 (d, J=7.8 Hz, 1H), 7.40 (d, J=3.4 Hz, 1H), 7.31-7.19 (m, 7H), 7.12 (ddd, J=0.9, 7.1, 8.1 Hz, 1H), 6.98 (m, 2H), 6.85 (m, 1H), 6.73 (dd, J=1.5, 3.9 Hz, 1H), 6.41 (d, J=3.4 Hz, 1H), 6.06 (dd, J=2.5, 3.7 Hz, 1H), 5.94 (d, J=9.2 Hz, 1H), 5.77 (t, J=9.3 Hz, 1H), 5.06 (dd, J=7.3, 11.8 Hz, 1H), 4.97 (dd, J=8.3, 11.8 Hz, 1H), 4.89 (q, J=8.9 Hz, 1H), 4.76 (dd, J=7.3, 11.8 Hz, 1H), 4.63 (dd, J=8.3, 11.8 Hz, 1H), 3.98-3.91 (m, 2H), 3.82 (dd, J=5.4, 12.0 Hz, 1H), 3.78 (ddd, J=1.8, 5.3, 9.7 Hz, 1H).

### Example 2. Step 4

### (2R,3R,4S,5R,6R)-5-hydroxy-6-(hydroxymethyl)-2-(1H-indol-1-yl)-4-(phosphonooxy)tetrahydro-2H-pyran-3-yl 1H-pyrrole-2-carboxylate (26)

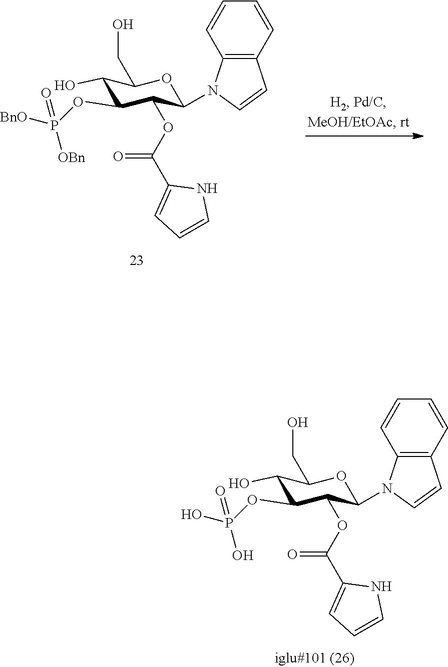

To a 1:1 mixture of MeOH/EtOAc (v/v, 2 mL) 23 (28.4 mg, 0.0449 mmol, 1.0 equiv.) and Pd/C (10% w/w) (23 mg) were added. The reaction mixture was purged with argon for 2 min, subjected to H₂ for 1 hr, at room temperature, and again purged with argon for 2 min. The reaction mixture was filtered through Celite and concentrated in vacuo. The residue was purified by reversed-phase flash chromatography with a C18 column using a gradient of 0-60% ACN in H₂O (with 0.1% formic acid), which afforded iglu #101 (26, 9.2 mg, 45%) as a clear oil. See Table S3 for NMR spectroscopic data of iglu #101 (26).

### Example 3. Step 1

### (6aR,8R,9R,10R,10aS)-10-hydroxy-8-(1H-indol-1-yl)-2,2,4,4-tetraisopropyl-hexahydropyrano[3,2-f][1,3,5,2,4]trioxadisilocin-9-yl 2-((tert-butoxycarbonyl)-amino)benzoate (18)

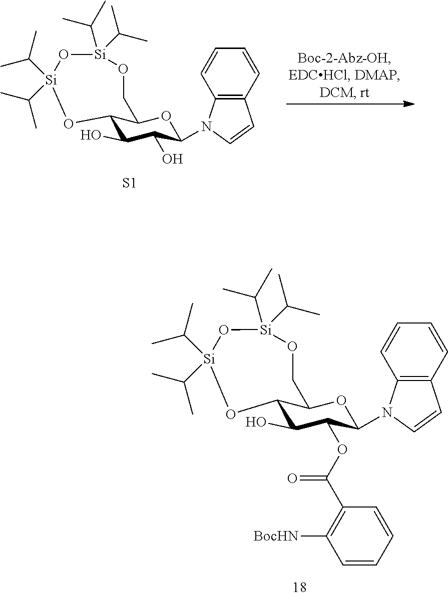

To a stirred solution of Boc-2-Abz-OH (24.0 mg, 0.101 mmol, 1.0 equiv.) in DCM, EDC·HCl (38.7 mg, 0.202 mmol, 2.0 equiv.) was added. The mixture was stirred at room temperature for 30 min, and S1 (63.0 mg, 0.121 mmol, 1.2 equiv.) and DMAP (30.8 mg, 0.252 mmol, 2.5 equiv.) were added. The reaction mixture was stirred at room temperature for 4 hr. The reaction mixture was concentrated in vacuo followed by flash column chromatography on silica using a gradient of 5-90% EtOAc in hexanes, which afforded 18 (21.6 mg, 29%). ¹H NMR (500 MHz, chloroform-d): δ (ppm) 9.76 (s, 1H), 8.33 (dd, J=0.8, 8.6 Hz, 1H), 7.69 (dd, J=1.6, 8.2 Hz, 1H), 7.47 (d, J=8.3 Hz, 1H), 7.41 (ddd, J=1.3, 1.6, 7.9 Hz, 1H), 7.25 (d, 1H), 7.18 (ddd, J=1.0, 1.0, 7.7 Hz, 1H), 7.07 (ddd, J=0.8, 0.8, 7.5 Hz, 1H), 6.84 (ddd, J=1.0, 1.0, 7.7 Hz, 1H), 6.47 (d, J=3.4 Hz, 1H), 5.71-5.64 (m, 2H), 4.16 (dd, J=1.9, 12.6 Hz, 1H), 4.14-4.09 (m, 2H), 4.03 (dd, J=1.1, 12.6 Hz, 1H), 3.55 (m, 1H), 1.49 (s, 9H), 1.18-1.02 (m, 28H).

### Example 3. Step 2

### (6aR,8R,9R,10R,10aR)-10-((bis(benzyloxy)phosphoryl)oxy)-8-(1H-indol-1-yl)-2,2,4,4-tetraisopropylhexahydropyrano[3,2-f][1,3,5,2,4]trioxadisilocin-9-yl 2-((tert-butoxycarbonyl)amino)benzoate (21)

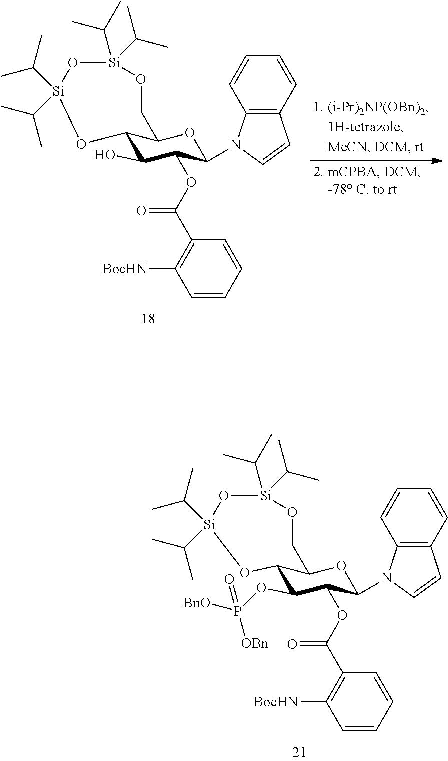

To a solution of 18 (21.6 mg, 0.0292 mmol, 1.0 equiv.) in 0.8 mL DCM was added dibenzyl N,N-diisopropylphosphoramidite (29 μL, 0.0875 mmol, 3.0 equiv.) and 1H-tetrazole (0.45 M in ACN, 194 μL, 0.0875 mmol, 3.0 equiv.). The reaction mixture was stirred at room temperature for 45 min. Then the solution was cooled to −78° C. under argon before adding 3-chloroperoxy benzoic acid (mCPBA, ~77%, 20 mg, 0.364 mmol, 3.0 equiv.). The solution was stirred at room temperature for 2-hr. The mixture was diluted with DCM and washed with sat. aq. NaHCO₃, dried with Na₂SO₄, and concentrated in vacuo. Flash column chromatography on silica using a gradient of 5-90% EtOAc in hexanes afforded 21 (25.5 mg, 87%). ¹H NMR (500 MHz, chloroform-d): δ (ppm) 9.48 in (s, 1H), 8.25 (d, J=8.4 Hz, 1H), 7.85 (dd, J=1.1, 8.0 Hz, 1H), 7.50 (d, J=8.0 Hz, 1H), 7.48 (d, J=8.5 Hz, 1H), 7.39 (m, 1H), 7.36-7.33 (m, 2H), 7.25-7.09 (m, 8H), 7.05 (ddd, J=0.7, 7.6, 7.8 Hz, 1H), 6.95 (m, 2H), 6.88 (ddd, J=0.7, 7.6, 8.4 Hz, 1H), 6.43 (d, J=3.3 Hz, 1H), 5.75 (d, J=9.1 Hz, 1H), 5.61 (d, J=9.0 Hz, 1H), 4.90-4.82 (m, 2H), 4.77 (dd, J=7.2, 11.6 Hz, 1H), 4.69 (dd, J=8.3, 11.6 Hz, 1H), 4.46 (t, J=11.2 Hz, 1H), 4.31 (t, J=9.2 Hz, 1H), 4.20 (dd, J=1.3, 12.7 Hz, 1H), 4.05 (dd, J=1.0, 12.7 Hz, 1H), 3.53 (d, J=9.3 Hz, 1H), 1.41 (s, 9H), 1.32-1.26 (m, 7H), 1.19 (d, J=6.8 Hz, 3H), 1.17 (d, J=6.8 Hz, 3H), 1.04-1.00 (m, 6H), 0.95 (d, J=6.8 Hz, 3H), 0.89 (d, J=6.8 Hz, 3H), 0.88 (d, J=7.1 Hz, 3H). ¹³C NMR (125 MHz, chloroform-d): 171.3, 167.3, 152.7, 142.6, 136.4, 135.5, 135.4, 135.0, 134.97, 134.92, 131.4, 129.3, 128.3, 128.2, 128.1, 127.41, 127.38, 125.2, 122.6, 121.4, 121.2, 120.8, 118.5, 113.9, 109.8, 104.4, 80.4, 79.9, 69.38, 69.34, 68.13, 68.09, 68.06, 59.2, 31.7, 28.4, 22.8, 21.2, 18.3, 17.43, 17.42, 17.39, 17.2, 17.0, 14.3, 14.2, 13.5, 13.3, 12.9, 12.6.

### Example 3. Step 3

### (2R,3R,4S,5R,6R)-4-((bis(benzyloxy)phosphoryl)oxy)-5-hydroxy-6-(hydroxymethyl)-2-(1H-indol-1-yl)tetrahydro-2H-pyran-3-yl 2-((tert-butoxycarbonyl)amino)benzoate (24)

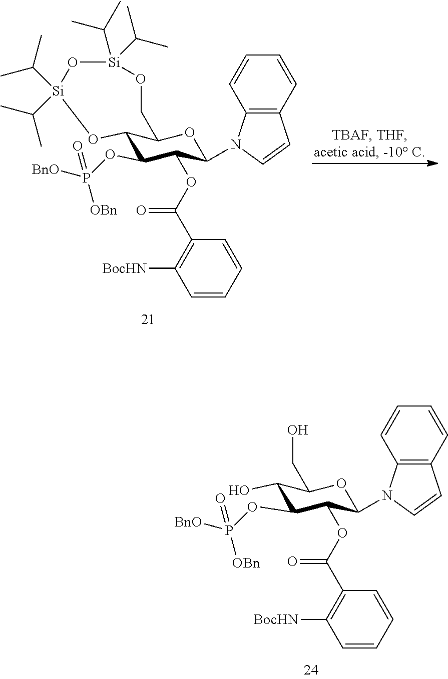

To a solution of 21 (25.5 mg, 0.0255 mmol, 1.0 equiv.) in 1 mL THF was added acetic acid (4.4 μL, 0.0765 mmol, 3.0 equiv.), and the mixture was cooled to −10° C. To the solution was added tetrabutylammonium fluoride (1M in THF, 77 μL, 0.0765 mmol, 3.0 eq) and the resulting mixture stirred for 1.4 hr. Subsequently, acetic acid (10 μL, 0.175 mmol, 6.8 equiv.) was added and the mixture concentrated in vacuo. Flash column chromatography on silica using a gradient of 5-20% MeOH in DCM afforded 24 (17.1 mg, 88%), containing about 20% of dibenzyl diiylphosphoramidite as an impurity. ¹H NMR (600 MHz, chloroform-d): δ (ppm) 9.64 (s, 1H), 8.33 (dd, J=1.1, 8.6 Hz, 1H), 7.71 (dd, J=1.6, 8.1 Hz, 1H), 7.54 (dt, J=0.9, 7.9 Hz, 1H), 7.43-7.39 (m, 2H), 7.36-7.34 (m, 2H), 7.30 (d, J=2.2 Hz, 1H), 7.30-7.27 (m, 2H), 7.24 (m, 1H), 7.21 (m, 1H), 7.14 (m, 2H), 7.10 (ddd, J=0.9, 7.5, 8.0 Hz, 1H), 7.03 (m, 2H), 6.80 (dt, J=1.1, 7.6 Hz, 1H), 6.51 (d, J=3.4 Hz, 1H), 5.80 (t, J=9.3 Hz, 1H), 5.68 (d, J=9.3 Hz, 1H), 4.96 (dd, J=8.2, 11.7 Hz, 1H), 4.83 (dd, J=7.9, 11.7 Hz, 1H), 4.80 (d, J=8.7 Hz, 1H), 4.68 (dt, J=7.2, 9.0 Hz, 1H), 4.02-3.94 (m, 2H), 3.88 (dd, J=5.1, 12.1 Hz, 1H), 3.72 (ddd, J=3.3, 5.1, 9.6 Hz, 1H), 1.44 (s, 9H). ¹³C NMR (125 MHz, chloroform-d): 175.4, 170.0, 152.5, 142.4, 136.3, 135.24, 135.19, 135.0, 134.93, 134.87, 130.7, 129.2, 128.69, 128.68, 128.62, 128.58, 127.8, 124.7, 122.5, 121.3, 121.1, 120.8, 118.6, 112.9, 109.8, 104.4, 83.1, 82.3, 82.2, 80.7, 78.6, 70.85, 70.81, 70.16, 70.11, 70.08, 70.04, 69.79, 69.78, 61.9, 50.6, 28.3.

### Example 3. Step 4

### (2R,3R,4S,5R,6R)-4-((bis(benzyloxy)phosphoryl)oxy)-5-hydroxy-6-(hydroxymethyl)-2-(1H-indol-1-yl)tetrahydro-2H-pyran-3-yl 2-aminobenzoate (27)

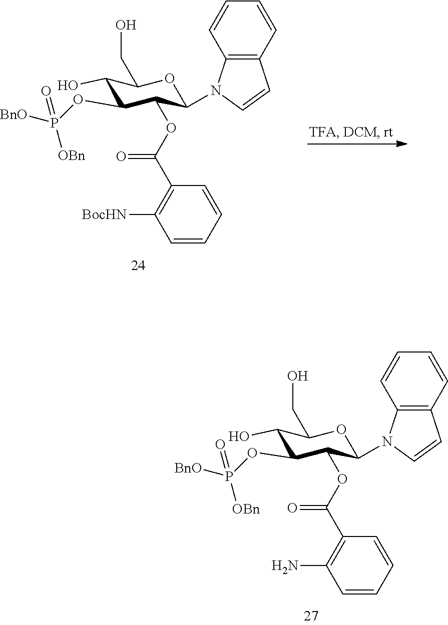

To a solution of 24 (17.1 mg, 0.0226 mmol, 1.0 equiv.) in 1.5 mL DCM was added TFA (0.1 mL, 1.31 mmol, 58 equiv.). The reaction mixture was stirred at room temperature for 20 min and then concentrated in vacuo. Flash column chromatography on silica using a gradient of 0-20% MeOH in DCM afforded 27 (14.7 mg, 99%), containing 27% of dibenzyl diisopropylphosphoramidite as impurity. ¹H NMR (600 MHz, DMSO-d₄): δ (ppm) 7.72 (d, J=8.4 Hz, 1H), 7.61 (dd, J=1.4, 8.2 Hz, 1H), 7.48 (d, J=3.4 Hz, 1H), 7.45 (d, J=7.8 Hz, 1H), 7.39-7.19 (m, 14H, with impuritiy), 7.13 (dt, J=1.7, 7.1 Hz, 1H), 7.00 (t, J=7.6 Hz, 1H), 6.97 (m, 2H), 6.61 (d, J=8.4 Hz, 1H), 6.43 (d, J=3.3 Hz, 1H), 6.37 (dt, J=1.0, 7.5 Hz, 1H), 6.20 (d, J=9.2 Hz, 1H), 5.90 (br, 1H), 5.76 (d, J=9.2 Hz, 1H), 4.94 (m, 2H), 4.70 (dd, J=7.1, 12.0 Hz, 1H), 4.56 (dd, J=8.0, 12.0 Hz, 1H), 3.85-3.73 (m, 3H), 3.60 (dd, J=5.6, 12.3 Hz, 1H).

### Example 3. Step 5

### (2R,3R,4S,5R,6R)-5-hydroxy-6-(hydroxymethyl)-2-(1H-indol-1-yl)-4-(phosphonooxy)tetrahydro-2H-pyran-3-yl 2-aminobenzoate (iglu #401, 28)

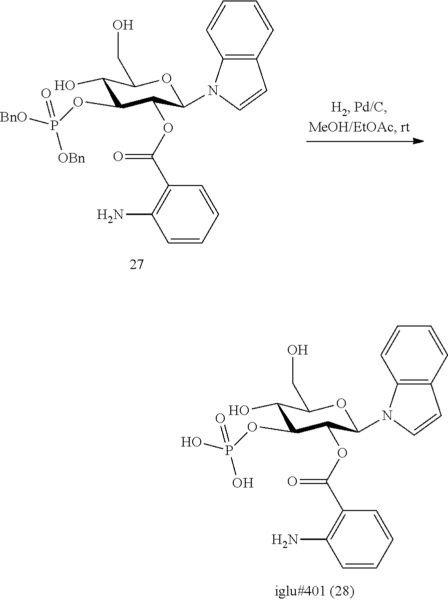

To a 1:1 mixture of MeOH/EtOAc (v/v, 2 mL) 27 (14.7 mg, 0.0223 mmol, 1.0 equiv.) and Pd/C (10% w/w) (14 mg) were added. The reaction mixture was purged with argon for 2 min, subjected to H₂ for 1 hr at room temperature, and again purged with argon for 2 min. The reaction mixture was filtered through Celite and concentrated in vacuo. The residue was purified by reversed-phase flash chromatography with a C18 column using a gradient of 0-60% ACN in H₂O (with 0.1% formic acid), which afforded iglu #401 (28, 1.6 mg, 15%) as a clear oil. See Table S2 for NMR spectroscopic data of iglu #401 (28).

### Example 4

### 4-((2-(5-hydroxy-1H-indol-3-yl)ethyl)amino)-4-oxobutanoic acid (14)

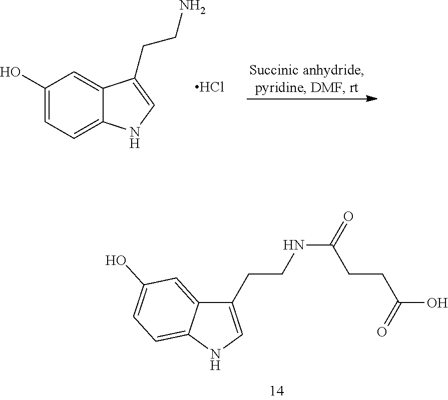 [SMILES: NCCc1c[nH]c2ccc(O)cc12 ; Cl ; O=C(O)CCC(=O)NCCc1c[nH]c2ccc(O)cc12]

To a solution of serotonin hydrochloride (128.1 mg, 0.602 mmol, 1.0 equiv.) in DMF (6 mL) was added succinic anhydride (78.3 mg, 0.783 mmol, 1.3 equiv.) and pyridine (0.6 mL). The mixture was stirred at room temperature for 24 hours and concentrated in vacuo. Flash column chromatography on silica using a gradient of 0-50% MeOH in DCM afforded 14 (165.4 mg, 99%) as clear oil. ¹H NMR (500 MHz, methanol-d₄): δ (ppm) 7.16 (d, J=8.6 Hz, 1H), 6.98 (s, 1H), 6.96 (d, J=2.3 Hz, 1H), 6.69 (dd, J=2.3, 8.6 Hz, 1H), 3.42 (t, J=7.2 Hz, 2H), 2.83 (t, J=7.2 Hz, 2H), 2.57 (t, J=7.0 Hz, 2H), 2.43 (t, J=7.0 Hz, 2H). ¹³C NMR (125 MHz, methanol-d₄): δ (ppm) 176.3, 174.3, 151.0, 132.9, 129.3, 124.3, 112.7, 112.4, 112.3, 103.5, 41.3, 31.5. 30.2, 26.1. HRMS (ESI) m/z calcd for C₁₄H₁₆N₂O₄ [M−H]⁻ 275.1037, found 275.1043.

### Example 5

### N-(2-(5-hydroxy-1H-indol-3-yl)ethyl) acetamide-1-¹³C (28)

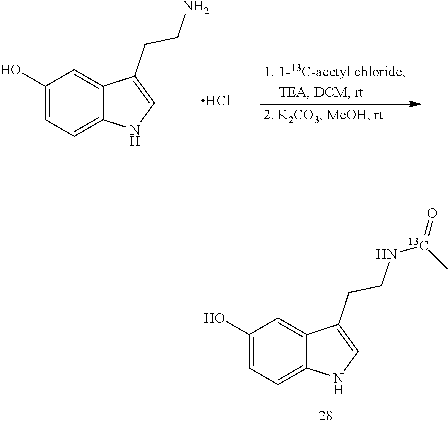 [SMILES: NCCc1c[nH]c2ccc(O)cc12 ; Cl ; C[13C](=O)NCCc1c[nH]c2ccc(O)cc12]

To a suspension of serotonin hydrochloride (132 mg, 0.621 mmol, 1.0 equiv.) in DCM (5 mL) was added TEA (433 μL, 3.10 mmol, 5.0 equiv.). The stirred mixture was cooled to 0° C. before 1-13C-acetyl chloride (93 μL, 1.30 mmol, 2.1 equiv.) was added. The mixture was slowly warmed to room temperature and stirred for 24 hours. The reaction mixture was then diluted with DCM, the organics were washed with water, dried with Na₂SO₄, and concentrated in vacuo. Crude intermediates were dissolved in MeOH (10 mL), and K₂CO₃ (85.8 mg, 0.621 mmol, 1.0 equiv.) was added. The reaction was stirred at room temperature for 2 hours and concentrated to 2 mL in vacuo. The residue was diluted with water and extracted with EtOAc twice. The organics were separated, washed with brine, and dried with Na₂SO₄. Flash column chromatography on silica using a gradient of 0-50% MeOH in DCM afforded 28 (98.0 mg, 72%) as light-yellow oil. ¹H NMR (600 MHz, methanol-d₄): δ (ppm) 7.15 (dd, J=0.6, 8.6 Hz, 1H), 6.99 (s, 1H), 6.93 (dd, J=0.6, 2.4 Hz, 1H), 6.66 (dd, J=2.4, 8.6 Hz, 1H), 3.42 (ddd, J=3.7, 7.3, 8.2 Hz, 2H), 2.85 (dt, J=0.6, 7.3 Hz, 2H), 1.91 (d, J=6.1 Hz, 3H). ¹³C NMR (125 MHz, methanol-d₄): δ (ppm) 175.9 (12C), 173.4 (13C), 151.1, 133.1, 129.5, 124.2, 112.6, 112.4, 103.5, 41.4, 26.2, 22.6 (d, J=50.3 Hz). HRMS (ESI) m/z calcd for C₁₁¹³CH₁₄N₂O₂ [M+H]⁺ 220.1161, found 220.1160.

### Example 6. Steps 1 and 2

### N-(2-(5-(((2R,3R,4S,5R,6R)-3,4,5-tris(benzyloxy)-6-((benzyloxy)methyl)tetrahydro-2H-pyran-2-yl)oxy)-1H-indol-3-yl)ethyl) acetamide (45)

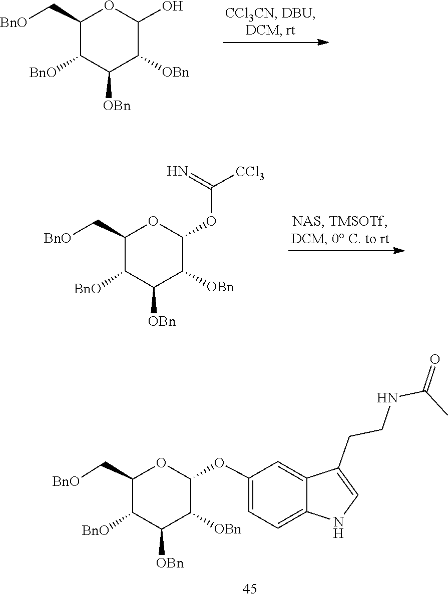

To a solution of 2,3,4,6-tetra-O-benzyl-D-glucopyranose (412 mg, 0.761 mmol, 1.0 equiv.) in DCM (2 mL) was added trichloroacetonitrile (152 μL, 1.52 mmol, 2.0 equiv.) and DBU (21 μL, 0.152 mmol, 0.2 equiv.) under argon. The mixture was stirred at room temperature for 1.5 hours and concentrated in vacuo. Flash column chromatography on silica using a gradient of 25% ethyl acetate in hexanes afforded intermediate 44 (502.4 mg, 97%) as clear oil. A well-stirred solution of 44 (502.4 mg, 0.745 mmol, 2.0 equiv.) and N-acetylserotonin (806 mg, 0.368 mmol, 1.0 equiv.) in DCM (4 mL) and DMF (0.8 mL) was cooled to 0° C., followed by addition of TMSOTf (66 μL, 0.368 mmol, 1.0 equiv.), and the solution was allowed to warm to room temperature within 30 minutes. After stirring at 45° C. for 18 hours, the mixture was concentrated in vacuo. Flash column chromatography on silica using a gradient of 0-15% MeOH in DCM afforded 45 (59.7 mg, 22%) as clear oil. ¹H NMR (500 MHz, chloroform-d): δ (ppm) 7.41-7.26 (m, 20H), 7.17-7.14 (m, 2H), 7.04-7.01 (m, 2H), 5.50 (d, J=3.4 Hz, 1H), 5.44 (m, 1H), 5.08 (d, J=10.8 Hz, 1H), 4.90 (d, J=11.0 Hz, 1H), 4.88 (d, J=10.9 Hz, 1H), 4.81 (d, J=12.0 Hz, 1H), 4.72 (d, J=12.0 Hz, 1H), 4.57 (d, J=11.9 Hz, 1H), 4.50 (d, J=10.8 Hz, 1H), 4.41 (d, J=12.0 Hz, 1H), 4.25 (t, J=9.2 Hz, 1H), 4.03 (m, 1H), 3.78-3.71 (m, 3H), 3.62 (dd, J=1.9, 10.8 Hz, 1H), 3.53 (dt, J=6.2, 6.6 Hz, 2H), 2.87 (t, J=6.6 Hz, 2H), 1.90 (s, 3H). ¹³C NMR (125 MHz, chloroform-d): δ (ppm) 170.1, 151.2, 139.0, 138.4, 138.2, 138.0, 132.7, 128.62, 128.58, 128.54, 128.48, 128.19, 128.13, 128.05, 128.02, 127.87, 127.82, 127.78, 123.1, 114.3, 113.2, 111.9, 105.8, 96.8, 82.2, 80.0, 77.8, 76.0, 75.3, 75.5, 10.8, 68.7, 39.6, 25.4, 23.5.

### Example 6. Step 3

### N-(2-(5-(((2R,3R,4S,5S,6R)-3,4,5-trihydroxy-6-(hydroxymethyl)tetrahydro-2H-pyran-2-yl)oxy)-1H-indol-3-yl)ethyl) acetamide (36)

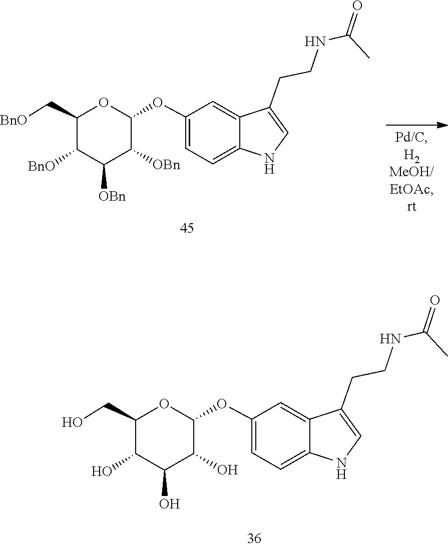 [SMILES: CC(=O)NCCc1c[nH]c2ccc(O[C@H]3O[C@H](COCc4ccccc4)[C@@H](OCc4ccccc4)[C@H](OCc4ccccc4)[C@H]3OCc3ccccc3)cc12 ; CC(=O)NCCc1c[nH]c2ccc(O[C@H]3O[C@H](CO)[C@@H](O)[C@H](O)[C@H]3O)cc12]

To a solution of 45 (59.2 mg, 0.080 mmol, 1.0 equiv.) in a mixture of MeOH and EtOAc (3 mL, v/v=1:1) was added Pd/C (10% w/w, 38 mg). The stirred reaction mixture was purged with argon for 5 minutes, flushed with hydrogen and then subjected to a hydrogen atmosphere for 2 hours at room temperature, and again purged with argon for 5 minutes. The mixture was filtered through Celite and concentrated in vacuo. affording 36 as clear oil (29.8 mg, 98%). HRMS (ESI) m/z calcd for C₁₈H₂₄N₂O₇ [M+Na]⁺ 403.1476, found 403.1486.

### Example 7. Step 1. Synthesis of sngl #101 (37)

### N-(2-(5-hydroxyindolin-3-yl)ethyl) acetamide (46)

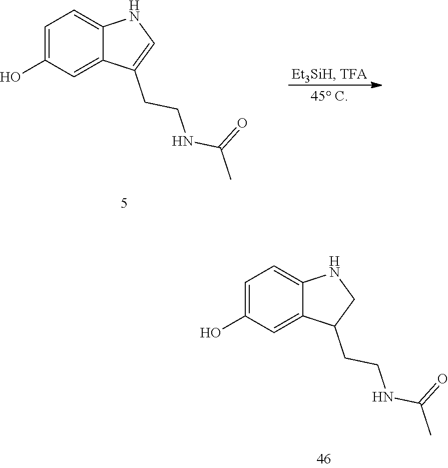 [SMILES: CC(=O)NCCc1c[nH]c2ccc(O)cc12 ; CC(=O)NCCC1CNc2ccc(O)cc21]

To a solution of N-acetylserotonin (210.2 mg, 0.963 mmol, 1.0 equiv.) in TFA (4 mL) was added triethylsilane (185 μL, 1.15 mmol, 1.2 equiv.). The mixture was stirred at 45° C. for 4 hours and concentrated in vacuo. Flash column chromatography on silica using a gradient of 0-40% MeOH in DCM afforded 46 (209.0 mg, 99%). ¹H NMR (500 MHz, methanol-d₄): δ (ppm) 7.17 (d, J=8.6 Hz, 1H), 6.79 (d, J=2.2 Hz, 1H), 6.74 (dd, J=2.2, 8.6 Hz, 1H), 3.95-3.88 (m, 1H), 3.62-3.55 (m, 1H), 3.49-3.42 (m, 2H), 3.29-3.20 (m, 2H), 2.02-1.94 (m, 1H), 1.88 (s, 3H), 1.72-1.63 (m, 1H). ¹³C NMR (125 MHz, methanol-d₄): δ (ppm) 173.5 (br), 160.2, 141.3, 128.5, 120.1, 116.6, 112.7, 52.5, 40.6, 38.0, 34.5, 22.5. HRMS (ESI) m/z calcd for C₁₂H₁₆N₂O [M+H]⁺ 221.1284, found 221.1272.

### Example 7. Step 2

### (2R,3R,4S,5R,6R)-2-(3-(2-acetamidoethyl)-5-acetoxyindolin-1-yl)-6-(acetoxymethyl)tetrahydro-2H-pyran-3,4,5-triyl triacetate (47)

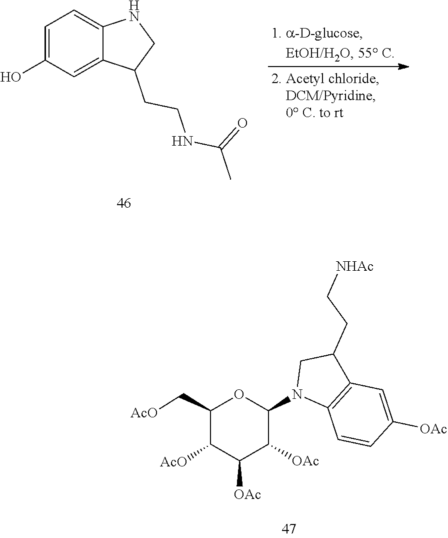 [SMILES: CC(=O)NCCC1CNc2ccc(O)cc21 ; CC(=O)NCCC1CN([C@@H]2O[C@H](COC(C)=O)[C@@H](OC(C)=O)[C@H](OC(C)=O)[C@H]2OC(C)=O)c2ccc(OC(C)=O)cc21]

To a solution of 46 (209 mg, 0.953 mmol, 1.0 equiv.) in TFA (1.5 mL) was added α-D-glucose (867 mg, 4.82 mmol, 5.0 equiv.). The mixture was refluxed for 2 hours and concentrated in vacuo. The crude intermediate was redissolved in pyridine (15 mL) and acetic anhydride (8 mL, 86.7 mmol, 90 equiv.) was added. The resulting mixture was stirred at room temperature for 1 hour and then diluted with water and extracted with DCM: MeOH (v/v=95:5) for three times. The combined organics were washed with sat. aq. NaHCO₃ and brine and dried with Na₂SO₄. Flash column chromatography on silica using a gradient of 0-30% isopropanol in toluene afforded 47 (mixture of diastereomers, 19.5 mg, 3.8%) as yellow oil. ¹H NMR (600 MHz, chloroform-d): δ (ppm) 6.86-6.78 (m, 2H), 6.52 (d, J=8.4 Hz, 0.5H), 6.50 (d, J=8.5 Hz, 0.5H), 5.67 (m, 0.5H), 5.57 (m, 0.5H), 5.33 (dt, J=6.7, 9.4 Hz, 1H), 5.23 (dt, J=8.2, 9.2 Hz, 1H), 5.07 (td, J=3.3, 9.7 Hz, 1H), 4.91 (d, J=10.0 Hz, 1H), 4.25 (ddd, J=5.0, 10.9, 12.4 Hz, 1H), 4.04 (ddd, J=2.4, 12.3, 17.5 Hz, 1H), 3.77-3.71 (m, 2H), 3.34-3.28 (m, 3H), 3.21 (m, 1H), 2.35 (s, 3H), 2.04 (d, J=1.7 Hz, 3H), 2.03 (d, J=1.7 Hz, 3H), 2.01-1.98 (6H), 1.94 (d, J=11.8 Hz, 3H), 1.76-1.62 (m, 2H). HRMS (ESI) m/z calcd for C₂₈H₃₆N₂O₁₂ [M+H]⁺ 593.2341, found 593.2299.

### Example 7. Step 3

### (2R,3R,4S,5R,6R)-2-(3-(2-acetamidoethyl)-5-acetoxy-1H-indol-1-yl)-6-(acetoxymethyl)tetrahydro-2H-pyran-3,4,5-triyl triacetate (48)

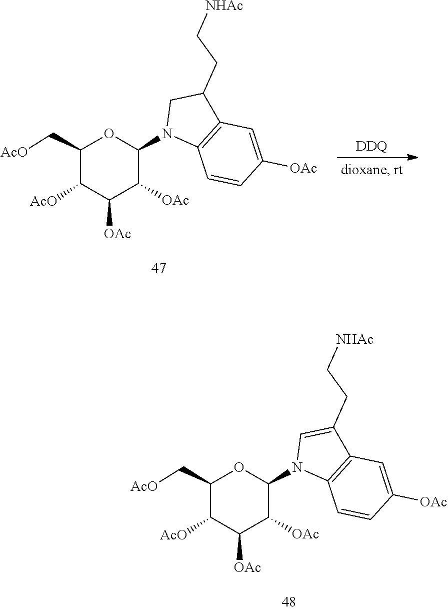

To a solution of 47 (19.5 mg, 0.0324 mmol, 1.0 equiv.) in 1,4-dioxane (1 mL) was added DDQ (8.8 mg, 0.039 mmol, 1.2 equiv.), and the mixture was stirred at room temperature. After 1.5 hours, the reaction mixture was cooled to 0° C. ice bath, diluted with sat. aq. NaHCO₃, and extracted with EtOAc for three times. Combined organics were washed with brine, dried with Na₂SO₄, and then concentrated in vacuo. Flash column chromatography on silica using 100% DCM afforded 48 (15.2 mg, 79%). ¹H NMR (600 MHz, chloroform-d): δ (ppm) 7.31 (d, J=8.9 Hz, 1H), 7.23 (d, J=2.1 Hz, 1H), 7.15 (s, 1H), 6.98 (dd, J=2.1, 8.9 Hz, 1H), 5.91 (m, 1H), 5.53 (d, J=9.0 Hz, 1H), 5.46 (t, J=9.5 Hz, 1H), 5.35 (t, J=9.4 Hz, 1H), 5.25 (t, J=9.8 Hz, 1H), 4.32 (dd, J=5.0, 12.6 Hz, 1H), 4.16 (dd, J=2.1, 12.6 Hz, 1H), 4.11 (q, J=7.2 Hz, 1H), 4.01 (ddd, J=2.2, 5.0, 10.2 Hz, 1H), 3.67 (m, 1H), 3.42 (m, 1H), 2.93 (m, 1H), 2.81 (m, 1H), 2.31 (s, 3H), 2.084 (s, 3H), 2.078 (s, 3H), 2.02 (s, 3H), 1.94 (s, 3H), 1.55 (s, 3H). ¹³C NMR (125 MHz, chloroform-d): δ (ppm) 170.7, 170.6, 170.4, 170.1, 169.6, 169.2, 144.9, 134.6, 128.8, 123.6, 117.1, 115.7, 111.8, 109.8, 83.0, 75.0, 72.8, 71.5, 68.3, 62.0, 51.0, 39.1, 23.3, 21.3, 20.9, 20.73, 20.70, 20.2. HRMS (ESI) m/z calcd for C₂₈H₃₄N₂O₁₂ [M+H]⁺ 591.2184, found 591.2151.

### Example 7. Step 4

### N-(2-(5-hydroxy-1-((2R,3R,4S,5S,6R)-3,4,5-trihydroxy-6-(hydroxymethyl)tetrahydro-2H-pyran-2-yl)-1H-indol-3-yl)ethyl) acetamide (sngl #101, 37)

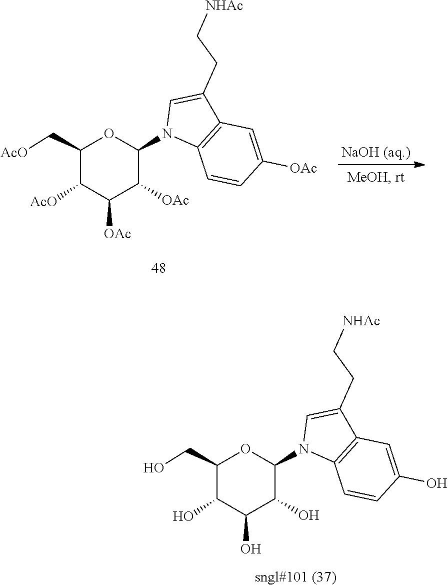 [SMILES: CC(=O)NCCc1cn([C@@H]2O[C@H](COC(C)=O)[C@@H](OC(C)=O)[C@H](OC(C)=O)[C@H]2OC(C)=O)c2ccc(OC(C)=O)cc12 ; CC(=O)NCCc1cn([C@@H]2O[C@H](CO)[C@@H](O)[C@H](O)[C@H]2O)c2ccc(O)cc12]

To a solution of 48 (15.2 mg, 0.0257 mmol, 1.0 equiv.) in MeOH (1.5 mL) was added 8% NaOH (0.3 mL). The mixture was stirred at room temperature for 25 min. and concentrated in vacuo. Flash column chromatography on silica using a gradient of 0-90% MeOH in DCM afforded 37 as clear oil (5.7 mg, 58%). HRMS (ESI) m/z calcd for C₁₈H₂₄N₂O₇ [M+Na]⁺ 403.1476, found 403.1471.

### Example 8. Step 1

### (2R,3R,4S,5S,6R)-2-fluoro-6-(hydroxymethyl)tetrahydro-2H-pyran-3,4,5-triol (38). 38 was prepared as previously described^{4,5}

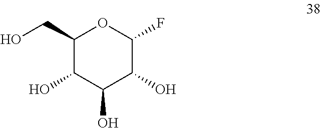 [SMILES: OC[C@H]1O[C@H](F)[C@H](O)[C@@H](O)[C@@H]1O]

### Example 8. Step 2

### N-(2-(5-(((2S,3R,4S,5S,6R)-3,4,5-trihydroxy-6-(hydroxymethyl)tetrahydro-2H-pyran-2-yl)oxy)-1H-indol-3-yl)ethyl) acetamide (sngl #1, 29)

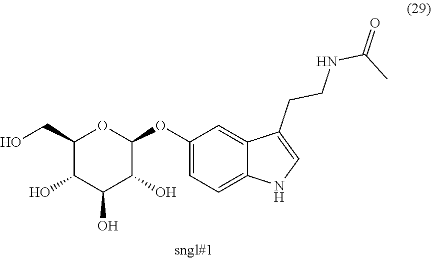 [SMILES: CC(=O)NCCc1c[nH]c2ccc(O[C@@H]3O[C@H](CO)[C@@H](O)[C@H](O)[C@H]3O)cc12]

To a 20 mL glass vial containing 38 (1.52 g, 8.35 mmol, 3 equiv.), N-acetylserotonin (607 mg, 2.78 mmol, 1.0 equiv.) and Ca(OH)₂ (618 mg, 8.35 mmol, 3 equiv.) was added water (3 mL). The reaction mixture was stirred vigorously for 35 minutes. The crude mixture was purified by reversed-phase flash chromatography with a C18 column using a gradient of 0-40% MeOH in H₂O, which afforded sngl #1 (29, 779.0 mg, 74%) as a white solid. HRMS (ESI) m/z calcd for C₁₈H₂₄N₂ NaO₇⁺ [M+Na]⁺ 403.1476, found 403.1485.

### Example 8. Step 3

### ((2R,3S,4S,5R,6S)-6-((3-(2-acetamidoethyl)-1H-indol-5-yl)oxy)-3,4,5-trihydroxytetrahydro-2H-pyran-2-yl)methyl 2-aminobenzoate (sngl #3, 31)

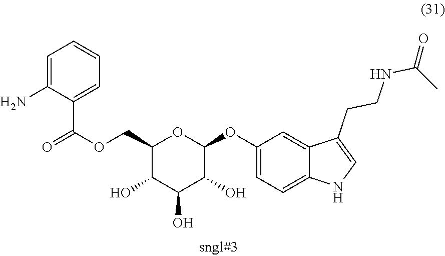 [SMILES: CC(=O)NCCc1c[nH]c2ccc(O[C@@H]3O[C@H](COC(=O)c4ccccc4N)[C@@H](O)[C@H](O)[C@H]3O)cc12]

To a mixture of DCM/DMF (3 mL, v/v=1:2) was added Boc-2-aminobenzoic acid (15.4 mg, 0.065 mmol, 1.2 equiv.) and EDC·HCl (31.2 mg, 0.163 mmol, 3.0 equiv.). The mixture was stirred at room temperature for 30 minutes, and DMAP (26.5 mg, 0.217 mmol, 4.0 equiv.) and sngl #1 (29, 20.6 mg, 0.0542 mmol, 1.0 equiv.) were added. After 5 days, the reaction mixture was concentrated in vacuo. Flash column chromatography on silica using a gradient of 0-30% MeOH in DCM afforded intermediate 49 (4.1 mg, 13%).

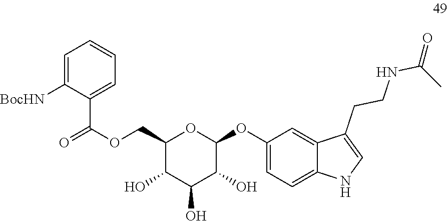 [SMILES: CC(=O)NCCc1c[nH]c2ccc(O[C@@H]3O[C@H](COC(=O)c4ccccc4NC(=O)OC(C)(C)C)[C@@H](O)[C@H](O)[C@H]3O)cc12]

Intermediate 49 was redissolved in DCM (1 mL), followed by slow addition of TFA (0.1 mL). The reaction mixture was stirred at room temperature for 1.5 hours and concentrated in vacuo. Preparative HPLC provided a pure sample of sngl #3 (31, 0.3 mg, 1.1%). HRMS (ESI) m/z calcd for C₂₅H₂₉N₃O₈ [M+H]⁺ 500.2027, found 500.2005.

### Example 8. Step 1

### N-(2-(5-(((6aR,8S,9R,10R,10aS)-9,10-dihydroxy-2,2,4,4-tetraisopropylhexahydropyrano[3,2-f][1,3,5,2,4]trioxadisilocin-8-yl)oxy)-1H-indol-3-yl)ethyl) acetamide (50)

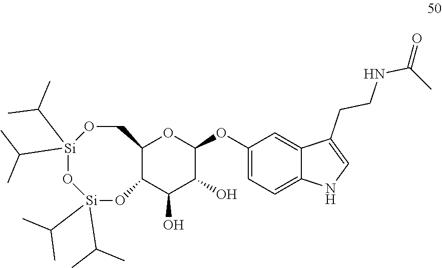 [SMILES: CC(=O)NCCc1c[nH]c2ccc(O[C@@H]3O[C@@H]4CO[Si](C(C)C)(C(C)C)O[Si](C(C)C)(C(C)C)O[C@H]4[C@H](O)[C@H]3O)cc12]

To a solution of sngl #1 (29, 194 mg, 0.511 mmol, 1.0 equiv.) in DMF was added imidazole (152 mg, 1.84 mmol, 4.4 equiv.) was cooled to 0° C. before TIPDSiCl₂ (228 μL, 0.713 mmol, 1.4 equiv.) was added. The reaction mixture was allowed to warm to room temperature over 1.5 hours and stirred for another 30 minutes. The mixture was then diluted with DCM and quenched with water. The organics were washed with sat. aq. NaHCO₃, dried with Na₂SO₄, and concentrated in vacuo. Flash column chromatography on silica using a gradient of 0-10% MeOH in DCM afforded 50 as a white solid (227.6 mg, 72%). ¹H NMR (500 MHz, chloroform-d): δ (ppm) 8.16 (s, 1H), 7.25-7.20 (m, 2H), 7.04-6.95 (m, 2H), 4.89 (d, J=7.3 Hz, 1H), 4.13 (d, J=11.9 Hz, 1H), 4.01 (d, J=12.5 Hz, 1H), 3.93 (t, J=8.9 Hz, 1H), 3.75-3.64 (m, 2H), 3.52 (m, 2H), 3.36 (m, 1H), 3.88 (m, 2H), 1.94 (s, 3H), 1.10-0.99 (m, 28H). HRMS (ESI) m/z calcd for C₃₀H₅₀N₂O₈Si₂, [M+H]⁺ 623.3178, found 623.3157.

### Example 8. Step 2

### (6aR,8S,9R,10R,10aS)-8-((3-(2-acetamidoethyl)-1H-indol-5-yl)oxy)-10-hydroxy-2,2,4,4-tetraisopropylhexahydropyrano[3,2-f][1,3,5,2,4]trioxadisilocin-9-yl benzyl carbonate (51)

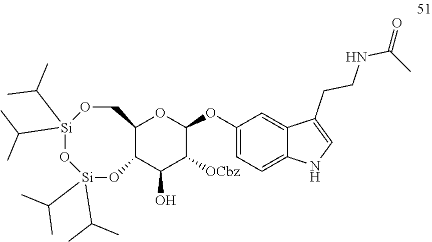

To a solution of 50 (227 mg, 0.365 mmol, 1.0 equiv.) in DCM was added DMAP (147 mg, 1.20 mmol, 3.3 equiv.) and DMF (50 μL). The mixture was cooled to 0° C. before added benzyl chloroformate (233 μL, 1.64 mmol, 4.5 equiv.). The reaction mixture was allowed to warm to room temperature within 30 minutes and stirred for another 1.3 hours. The mixture was diluted with DCM and then quenched with water. The aqueous layer was separated and extracted with DCM for three times. The combined organics were washed with sat. aq. NaHCO₃ and brine, dried with Na₂SO₄, and concentrated in vacuo. Flash column chromatography of the residue on silica using a gradient of 0-20% isopropanol in toluene afforded 51 as a white solid (196.1 mg, 66%). ¹H NMR (600 MHz, chloroform-d): δ (ppm) 8.22 (s, 1H), 7.40-7.37 (m, 2H), 7.36-7.31 (m, 3H), 7.19-7.16 (m, 2H), 6.99 (s, 1H), 6.82 (dd, J=2.2, 8.7 Hz, 1H), 5.64 (m, 1H), 5.26 (d, J=12.1 Hz, 1H), 5.21 (d, J=12.1 Hz, 1H), 4.97 (d, J=8.0 Hz, 1H), 4.93 (dd, J=8.7, 9.3 Hz, 1H), 4.12 (dd, J=1.9, 12.7 Hz, 1H), 4.05 (dd, J=1.2, 12.7 Hz, 1H), 3.98 (t, J=1.2, 9.3 Hz, 1H), 3.81 (t, J=1.2, 9.1 Hz, 1H), 3.51-3.47 (m, 2H), 3.34 (dt, J=1.2, 9.4 Hz, 1H), 2.83 (t, J=6.6 Hz, 2H), 1.88 (s, 3H), 1.14-1.01 (m, 28H). ¹³C NMR (125 MHz, chloroform-d): δ (ppm) 170.5, 155.1, 151.7, 135.2, 133.1, 128.74, 128.68, 128.48, 128.35, 127.8, 126.4, 114.5, 113.0, 111.8, 106.6, 101.5, 77.9, 76.7, 75.2, 70.2, 69.7, 60.9, 39.8, 25.2, 23.3, 17.57, 17.47, 17.43, 17.37, 17.33, 17.31, 17.25, 13.7, 13.3, 12.7, 12.6. HRMS (ESI) m/z calcd for C₃₈H₅₆N₂O₁₀Si₂ [M+H]⁺ 757.3546, found 757.3517.

### Example 8. Step 3

### (6aR,8S,9R,10R,10aR)-8-((3-(2-acetamidoethyl)-1H-indol-5-yl)oxy)-10-((bis(benzyloxy)phosphoryl)oxy)-2,2,4,4-tetraisopropylhexahydropyrano[3,2-f][1,3,5,2,4]trioxadisilocin-9-yl benzyl carbonate (52)

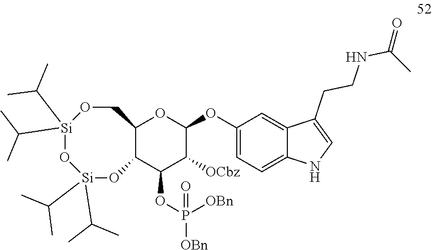

To a solution of 51 (145.8 mg, 0.188 mmol, 1.0 equiv.) in DCM was added dibenzyl N,N-diisopropylphosphoramidite (221 μL, 0.659 mmol, 3.5 equiv.) and 1H-tetrazole (0.45 M in ACN, 1.5 mL, 0.659 mmol, 3.5 equiv.). The reaction mixture was stirred at room temperature for 1 hour. The solution was cooled to −78° C. under argon, and m-CPBA (≤77%, 143.0 mg, 0.638 mmol, 3.4 equiv.) in DCM (1.5 mL) was added slowly to the reaction mixture. The solution was stirred at −78° C. for 0.5 hour, and slowly warmed to room temperature and reacted for another 1 hour, then was diluted with DCM and washed with 10% Na₂SO₄ twice, sat. aq. NaHCO₃, and brine, dried with Na₂SO₄, and concentrated in vacuo. Flash column chromatography on silica using a gradient of 5-100% EtOAc in hexanes afforded 52 as a white solid (141.3 mg, 74%). ¹H NMR (500 MHz, chloroform-d): δ (ppm) 8.31 (s, 1H), 7.36-7.27 (m, 15H), 7.22-7.18 (m, 2H), 7.04 (d, J=2.2 Hz, 1H), 6.78 (dd, J=2.2, 8.7 Hz, 1H), 5.53 (m, 1H), 5.23 (d, J=12.2 Hz, 1H), 5.13 (dd, J=8.0, 9.4 Hz, 1H), 5.08-4.91 (m, 6H), 4.61 (dt, J=8.6, 8.9 Hz, 1H), 4.20-4.14 (m, 2H), 4.09 (d, J=12.6 Hz, 1H), 3.59-3.47 (m, 2H), 3.33 (dt, J=1.7, 9.4 Hz, 1H), 2.87 (t, J=6.6 Hz, 2H), 1.92 (s, 3H), 1.16-0.99 (m, 28H). ¹³C NMR (125 MHz, chloroform-d): 170.2, 154.6, 151.7, 136.14, 136.08, 135.90, 135.85, 135.3, 133.2, 128.60, 128.57, 128.53, 128.49, 128.36, 128.12, 128.06, 127.8, 123.3, 114.6, 113.1, 111.8, 106.8, 101.6, 80.3 (d, J=6.5 Hz), 76.6 (d, J=4.6 Hz), 70.0, 69.6 (t, J=5.9 Hz), 68.7 (d, J=5.2 Hz), 60.9, 39.8, 25.3, 23.5, 17.54, 17.50, 17.46, 17.41, 17.36, 17.34, 17.28, 17.11, 13.35, 13.26, 12.97, 12.95. HRMS (ESI) m/z calcd for C₅₂H₆₉N₂O₁₃PSi₂ [M+H]⁺ 1017.4149, found 1017.4105.

### Example 8. Step 4

### (2S,3R,4S,5R,6R)-2-((3-(2-acetamidoethyl)-1H-indol-5-yl)oxy)-4-((bis(benzyloxy)phosphoryl)oxy)-5-hydroxy-6-(hydroxymethyl)tetrahydro-2H-pyran-3-yl benzyl carbonate (53)

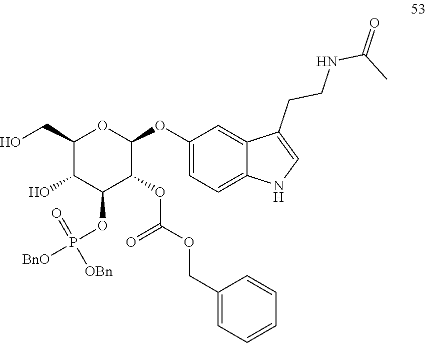 [SMILES: CC(=O)NCCc1c[nH]c2ccc(O[C@@H]3O[C@H](CO)[C@@H](O)[C@H](OP(=O)(OCc4ccccc4)OCc4ccccc4)[C@H]3OC(=O)OCc3ccccc3)cc12]

To a solution of 52 (141.3 mg, 0.139 mmol, 1.0 equiv.) in THF (6 mL) was added acetic acid (24 μL, 0.417 mmol, 3.0 equiv.). The solution was cooled to −10° C. before tetrabutylammonium fluoride solution (1M in THF, 417 μL, 00.417 mmol, 3.0 equiv.) was added. The reaction mixture was stirred for 1.5 hours in cold and concentrated in vacuo. Flash column chromatography on silica using a gradient of 0-15% MeOH in DCM afforded 53 as a white solid (92.3 mg, 86%). ¹H NMR (500 MHz, chloroform-d): δ (ppm) 8.32 (s, 1H), 7.36-7.21 (m, 15H), 7.14 (d, J=8.7 Hz, 1H), 6.96 (d, J=2.1 Hz, 1Hd), 6.71 (dd, J=2.1, 8.7 Hz, 1H), 5.82 (m, 1H), 5.12 (d, J=12.2 Hz, 1H), 5.10-4.94 (m, 7H), 4.49 (dt, J=7.2, 8.9 Hz, 1H), 3.99 (dd, J=2.8, 12.2 Hz, 1H), 3.84-3.74 (m, 2H), 3.55-3.46 (m, 2H), 3.40 (m, 1H), 2.91-2.77 (m, 2H), 1.89 (s, 3H). ¹³C NMR (125 MHz, chloroform-d): δ (ppm) 171.1, 154.5, 151.2, 135.0, 133.1, 128.81, 128.75, 128.72, 128.71, 128.69, 128.66, 123.4, 114.2, 113.1, 111.8, 106.9, 100.6, 81.7 (d, J=5.6 Hz), 76.17, 76.0 (d, J=6.2 Hz), 70.4, 70.25 (d, J=6.0 Hz), 70.17, 70.13 (d, J=6.0 Hz), 62.3, 40.3, 25.4, 23.4. HRMS (ESI) m/z calcd for C₄₀H₄₃N₂O₁₂P [M−H]⁻ 773.2481, found 773.2488.

### Example 8. Step 5

### (2S,3R,4S,5R,6R)-2-((3-(2-acetamidoethyl)-1H-indol-5-yl)oxy)-3,5-dihydroxy-6-(hydroxymethyl)tetrahydro-2H-pyran-4-yl dihydrogen phosphate (30)

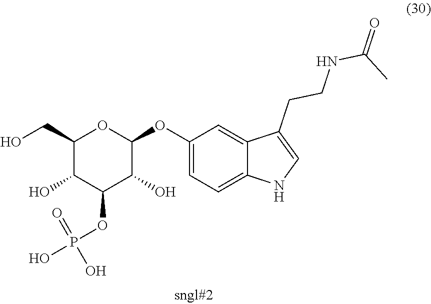 [SMILES: CC(=O)NCCc1c[nH]c2ccc(O[C@@H]3O[C@H](CO)[C@@H](O)[C@H](OP(=O)(O)O)[C@H]3O)cc12]

To a solution of 53 (26.9 mg, 0.0347 mmol, 1.0 equiv.) in a mixture of MeOH and EtOAc (2 mL, v/v=1:1) was added Pd/C (10% w/w, 18 mg). The reaction mixture was purged with argon for 5 minutes, flushed with hydrogen, and then subjected to hydrogen atmosphere for 1.5 hours at room temperature, and subsequently again purged with argon for 5 minutes. The mixture was filtered through Celite and concentrated in vacuo. The crude mixture was purified by reversed-phase flash chromatography with a C18 column using a gradient of 0-10% ACN in H₂O with 0.1% formic acid, which afforded sngl #2 as a clear oil (30, 9.3 mg, 58%). HRMS (ESI) m/z calcd for C₁₈H₂₅N₂O₁₀P [M−H]⁻ 459.1174, found 459.1185.

### Example 9. Step 1

### ((2R,3R,4S,5R,6S)-6-((3-(2-acetamidoethyl)-1H-indol-5-yl)oxy)-5-(((benzyloxy) carbonyl)oxy)-4-((bis(benzyloxy)phosphoryl)oxy)-3-hydroxytetrahydro-2H-pyran-2-yl)methyl 2-((tert-butoxycarbonyl)amino)benzoate (54)

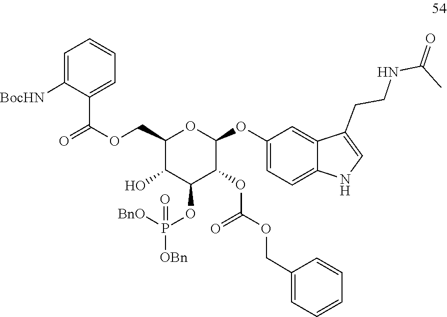 [SMILES: CC(=O)NCCc1c[nH]c2ccc(O[C@@H]3O[C@H](COC(=O)c4ccccc4NC(=O)OC(C)(C)C)[C@@H](O)[C@H](OP(=O)(OCc4ccccc4)OCc4ccccc4)[C@H]3OC(=O)OCc3ccccc3)cc12]

To a mixture of dry DCM/DMF (2 mL, v/v=100:1) was added Boc-2-aminobenzoic acid (70.7 mg, 0.298 mmol, 2.5 equiv.) and EDC·HCl (68.4 mg, 0.444 mmol, 3.0 equiv.). The mixture was stirred at room temperature for 25 minutes, and DMAP (58.2 mg, 0.476 mmol, 4.0 equiv.) and 53 (92.3 mg, 0.119 mmol, 1.0 equiv.) were added. After 25 hours, the reaction mixture was concentrated in vacuo. Flash column chromatography on silica using a gradient of 0-12% MeOH in DCM afforded 54 as a white solid (72.0 mg, 61%). ¹H NMR (600 MHz, chloroform-d): δ (ppm) 8.42 (d, J=8.4 Hz, 1H), 7.97 (dd, J=1.1, 8.0 Hz, 1H), 7.49 (dd, J=1.1, 7.8 Hz, 1H), 7.33-7.15 (m, 16H), 7.09 (d, J=8.7 Hz, 1H), 7.00 (d, J=1.1 Hz, 1H), 6.91 (t, J=7.8 Hz, 1H), 6.82 (dd, J=2.0, 8.7 Hz, 1H), 5.61 (m, 1H), 5.15-4.95 (m, 8H), 4.71 (dd, J=2.0, 12.0 Hz, 1H), 4.56 (dd, J=6.1, 12.0 Hz, 1H), 4.49 (m, 1H), 3.84 (t, J=9.4 Hz, 1H), 3.77 (m, 1H), 3.49-3.44 (m, 2H), 2.75 (t, J=6.9 Hz, 2H), 1.90 (s, 3H), 1.50 (s, 9H). HRMS (ESI) m/z calcd for C₅₂H₅₆N₃O₁₅P [M+H]⁺ 994.3522, found 994.3489.

### Example 9. Step 2

### ((2R,3R,4S,5R,6S)-6-((3-(2-acetamidoethyl)-1H-indol-5-yl)oxy)-5-(((benzyloxy) carbonyl)oxy)-4-((bis(benzyloxy)phosphoryl)oxy)-3-hydroxytetrahydro-2H-pyran-2-yl)methyl 2-aminobenzoate (55)

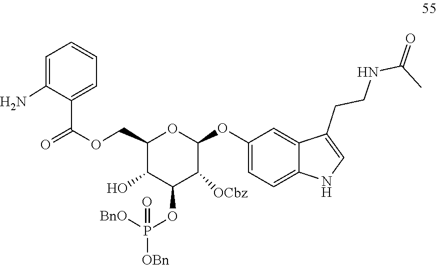

To a solution of 54 (72.0 mg, 72.5 μmol, 1.0 equiv.) in DCM (2 mL) was added TFA (200 μL). The yellow mixture was stirred at room temperature for 1 hour and turned purple. The reaction mixture was then concentrated in vacuo. Flash column chromatography on silica using a gradient of 0-10% MeOH in DCM afforded 55 (54.9 mg, 85%). ¹H NMR (600 MHz, acetone-d₆): δ (ppm) 7.89 (dd, J=1.5, 8.1 Hz, 1H), 7.40-7.24 (m, 17H), 7.22 (d, J=8.5 Hz, 1H), 7.15 (s, 1H), 6.84 (dd, J=2.3, 8.7 Hz, 1H), 6.80 (dd, J=0.6, 8.3 Hz, 1H), 6.56 (ddd, J=1.1, 7.1, 8.3 Hz, 1H), 5.32 (d, J=8.1 Hz, 1H), 5.24 (d, J=12.2 Hz, 1H), 5.17-5.03 (m, 6H), 4.84 (m, 1H), 4.75 (dd, J=1.2, 12.2 Hz, 1H), 4.53 (dd, J=5.6, 11.8 Hz, 1H), 4.10 (m, 1H), 4.03 (t, J=9.1 Hz, 1H), 3.50-3.39 (m, 2H), 2.83 (t, J=7.2 Hz, 2H), 1.87 (s, 3H). ¹³C NMR (125 MHz, acetone-d₆): δ (ppm) 168.4, 155.4, 152.4, 152.0, 137.1, 136.5, 132.0, 129.34, 129.32, 129.26, 129.18, 129.13, 129.07, 129.04, 128.84, 128.81, 128.68, 124.44, 117.3, 116.1, 113.8, 113.5, 112.5, 110.4, 106.8, 101.1, 81.6 (d, J=5.8 Hz), 77.1 (d, J=4.6 Hz), 74.6, 70.54, 70.46, 70.3 (d, J=5.5 Hz), 70.2 (d, J=5.5 Hz), 63.7, 40.4, 26.3, 23.0. HRMS (ESI) m/z calcd for C₄₇H₅₆N₃O₁₅P [M+H]⁺ 894.2998, found 894.2957.

### Example 9. Step 3

### ((2R,3R,4S,5R,6S)-6-((3-(2-acetamidoethyl)-1H-indol-5-yl)oxy)-3,5-dihydroxy-4-(phosphonooxy)tetrahydro-2H-pyran-2-yl)methyl 2-aminobenzoate (sngl #4, 32)

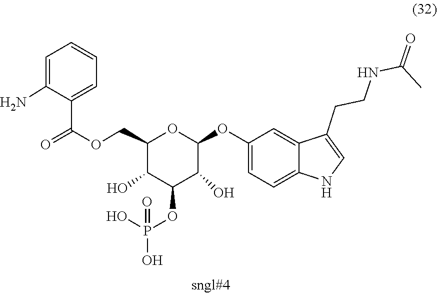 [SMILES: CC(=O)NCCc1c[nH]c2ccc(O[C@@H]3O[C@H](COC(=O)c4ccccc4N)[C@@H](O)[C@H](OP(=O)(O)O)[C@H]3O)cc12]

To a solution of 55 (54.9 mg, 61.4 μmol, 1.0 equiv.) in a mixture of MeOH and EtOAc (2.5 mL, v/v=2:3) was added Pd/C (10% w/w) (32 mg). The reaction mixture was purged with argon for 5 minutes, flushed with hydrogen, and then subjected to hydrogen atmosphere for 3 hours at room temperature, and again purged with argon for 5 minutes. The mixture was filtered through Celite and concentrated in vacuo, affording sngl #4 (32, 33.4 mg, 94%). HRMS (ESI) m/z calcd for C₃₃H₃₆N₃O₁₃P, [M−H]⁻ 578.1545, found 578.1554.

### Synthesis of Glucosyladenine Derivatives

### Example 10. Step 1. N⁹-(β-glucopyranosyl)-N⁶-methyladenine (BC-2, maglu #3)

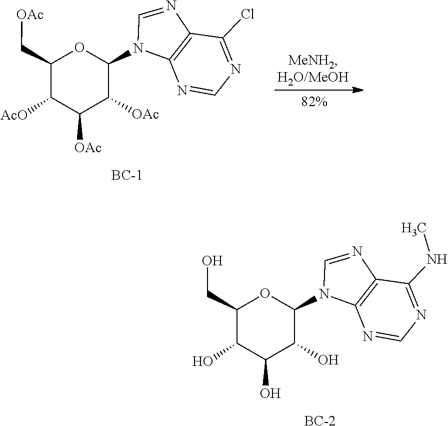

To BC-1 (503 mg, 0.62 mmol, 1.00 equiv.) in a high-pressure flask was added 15 mL of MeNH₂ (40% in H₂O) and 2 mL MeOH. The flask was sealed and heated to 100° C., at which the solution was stirred for 2 hr. The resulting solution was allowed to cool to room temp, at which a precipitate slowly formed, filtered, and washed with cold methanol/water, affording BC-2 (maglu #3, 266 mg, 82%) as a white solid. ¹H NMR (600 MHz, DMSO-d₆): δ 8.30 (s, 1H), 8.23 (br s, 1H), 7.70 (br s, 1H), 5.40 (d, J=9.4 Hz, 1H), 5.31 (d, J=5.8 Hz, 1H), 5.28 (d, J=4.6 Hz, 1H), 5.14 (d, J=5.4 Hz, 1H), 4.59 (t, J=5.9 Hz, 1H), 3.99 (td, J=9.1, 5.8 Hz, 1H), 3.70 (ddd, J=11.7, 5.7, 1.7 Hz, 1H), 3.43 (dt, J=11.9, 6.1 Hz, 1H), 3.41-3.34 (m, 2H), 3.24 (td, J=9.2, 5.6 Hz, 1H), 2.95 (br s, 3H). ¹³C NMR (126 MHz, DMSO-d₆): δ 155.0, 152.6, 139.4, 82.8, 80.0, 77.3, 71.3, 69.8, 60.9, 29.7. HRMS (ESI) m/z: [M+H]⁺ calcd for C₁₂H₁₈N₅O₅ 312.1302; found 312.1290.

### Example 11. Step 1. N⁹—(β-glucopyranosyl) adenine (BC-3)

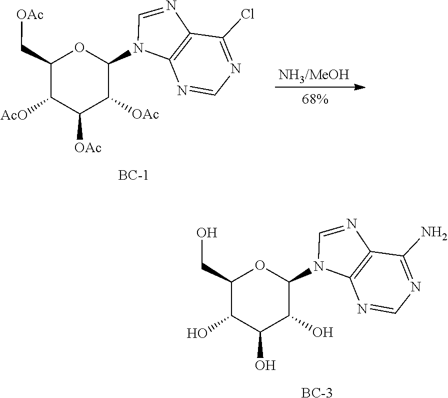

To BC-1 (1.00 g, 2.06 mmol, 1.00 equiv.) in a high-pressure flask was added 5 mL of MeOH and methanolic ammonia (7N, 29 mL, 206 mmol, 100 equiv.). The flask was sealed and heated to 100° C., at which the resulting yellow solution was stirred for 8 hr. The solution was transferred to a round-bottom flask and concentrated to dryness in vacuo. The reaction crude was then re-dissolved in MeOH upon heating, silica gel (11 g) was added, and the mixture was concentrated to dryness in vacuo (for dry-loading). Flash column chromatography on silica using a gradient of 30-60% MeOH in DCM was performed, affording BC-3 (420 mg, 68%) as an off-white power. ¹H NMR (600 MHz, DMSO-d₆): δ 8.31 (s, 1H), 8.14 (s, 1H), 7.23 (s, 2H), 5.39 (d, J=9.4 Hz, 1H), 5.30 (d, J=5.8 Hz, 1H), 5.25 (d, J=4.7 Hz, 1H), 5.12 (d, J=5.6 Hz, 1H), 4.57 (t, J=5.9 Hz, 1H), 3.99 (td, J=9.1, 5.8 Hz, 1H), 3.70 (ddd, J=11.7, 5.7, 1.7 Hz, 1H), 3.43 (dt, J=11.9, 6.1 Hz, 1H), 3.41-3.34 (m, 2H), 3.24 (td, J=9.2, 5.6 Hz, 1H). ¹³C NMR (126 MHz, DMSO-d₆): δ 156.0, 152.6, 149.8, 139.7, 118.7, 82.8, 80.0, 77.3, 71.2, 69.8, 60.9. HRMS (ESI) m/z: [M+H]⁺ calcd for C₁₁H₁₆N₅O₅ 298.1146; found 298.1136.

### Example 12. Step 1. N⁹—(β-glucopyranosyl)-N¹-methyladenine (BC-4, maglu #1)

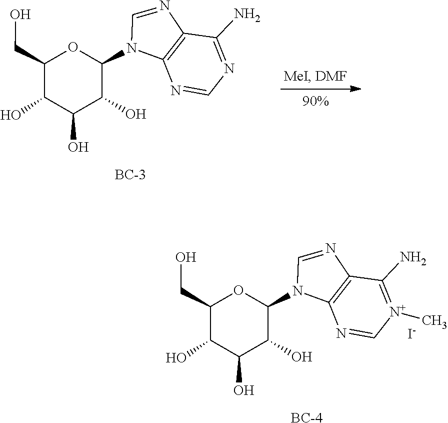

A solution of BC-3 (15 mg, 0.050 mmol, 1.00 equiv.) and Mel (12 μL, 0.193 mmol, 3.85 equiv.) in DMF (0.5 mL) was stirred for 48 hr at 40° C. The resulting yellow solution was concentrated to dryness in vacuo. Flash column chromatography on C18 using 100% H₂O (w/0.1% acetic acid) afforded maglu #1 (BC-4, 20 mg, 90%) as a white solid. maglu #1 was compared to the corresponding peak in C. elegans wildtype (N₂) endo-metabolome samples by HILIC-HRMS (Method C) and MS². ¹H NMR (500 MHz, methanol-d₄): 8.56 (s, 1H), 8.55 (s, 1H), 5.63 (d, J=9.3 Hz, 1H), 4.02 (t, J=9.0 Hz, 1H), 3.91 (s, 3H), 3.88 (d, J=12.1 Hz, 1H), 3.73 (dd, J=12.1, 5.3 Hz, 1H), 3.62-3.56 (m, 2H), 3.53 (t, J=9.1 Hz). ¹³C NMR (126 MHz, methanol-d₄): δ 152.7, 149.1, 148.7, 144.3, 120.3, 85.2, 81.3, 78.5, 73.7, 70.9, 62.3, 38.3. HRMS (ESI) m/z: [M+H]⁺ calcd for C₁₂H₁₈N₅O₅ 312.1302; found 312.1294.

### Example 13. Step 1. 6′-O, 4′-O-TIPDSi-N⁹—(β-glucopyranosyl) adenine (BC-5)

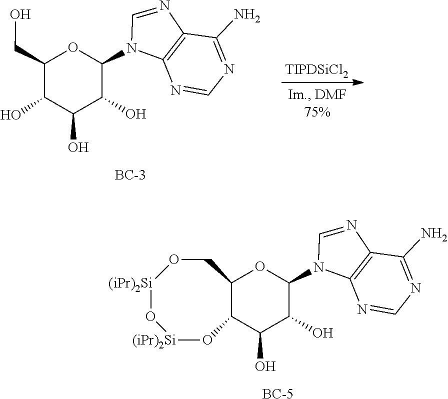

To a solution of BC-3 (350 mg, 1.18 mmol, 1.00 equiv.) in DMF (7 mL) at 0° C. was added TIPDSiCl₂ (560 μL, 1.75 mmol, 1.48 equiv.) and imidazole (362 mg, 5.32 mmol, 4.51 equiv.). The reaction mixture was stirred for 15 min at 0° C. and then diluted with DCM, followed by addition of H₂O. Organics were extracted 2× with DCM, combined, and then basified using sat. aq. NaHCO₃. The organic layer was collected and remaining organics were extracted 3× with a 2:1 mixture of DCM: EtOAc. Combined organics were dried using MgSO₄, filtered, and concentrated in vacuo. The reaction crude was then dissolved in a DCM/MeOH mixture, silica gel (2 g) was added, and the mixture was concentrated to dryness (for dry-loading). Flash column chromatography on silica using a gradient of 2.5-30% MeOH in DCM was performed, affording BC-5 (475 mg, 75%) as a white solid. ¹H NMR (500 MHz, methanol-d₄): δ 8.29 (s, 1H), 8.21 (s, 1H), 5.58 (d, J=9.4 Hz, 1H), 4.18 (dd, J=12.7, 2.2 Hz, 1H), 4.04 (t, J=9.2 Hz, 1H), 3.95 (t, J=9.1 Hz, 1H), 3.91 (dd, J=12.7, 0.8 Hz, 1H), 3.65 (t, J=9.0 Hz, 1H), 3.55 (dt, J=9.4, 1.8 Hz, 1H), 1.27-0.98 (m, 28H). ¹³C NMR (126 MHz, methanol-d₄): δ 157.4, 154.0, 151.2, 140.8, 120.0, 84.8, 80.8, 78.3, 73.7, 70.4, 62.1, 18.0, 17.8, 17.7, 17.6, 14.9, 14.5, 14.0, 13.8.

### Example 13. Step 2. Compounds BC-6 and BC-7

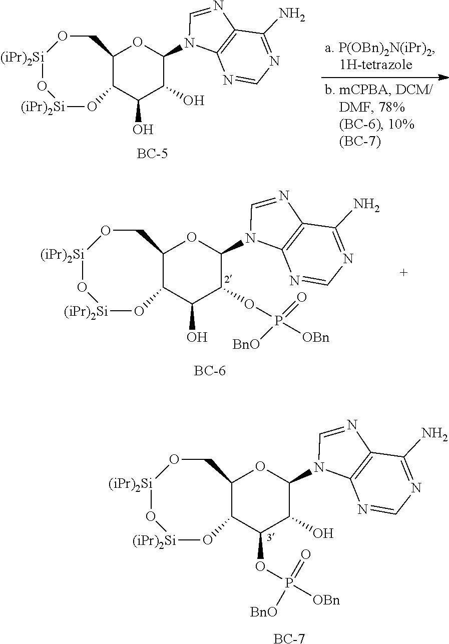

To a solution of BC-5 (260 mg, 0.48 mmol, 1.00 equiv.) in 1:1 DCM: DMF (16 mL) at 40° C. was added dibenzyl N,N-diisopropylphosphoramidite (0.58 mL, 1.73 mmol, 3.60 equiv.), and 1H-tetrazole (0.45 M in ACN, 3.20 mL, 1.44 mmol, 3.00 equiv.). The reaction mixture was stirred at 40° C. for 1 hr and then cooled to −78° C. after which mCPBA (77% max, 300 mg, 1.34 mmol, 2.79 equiv.) was added. The resulting mixture was stirred at −78° C. for 10 min, and was then quenched with the addition of sat. aq. NaHCO₃ (3 mL) after which H₂O (10 mL) and DCM (50 mL) were added. The organic layer was washed 1× with sat. aq. NaHCO₃ (10 mL total) and collected and the aqueous layer was extracted 2× with DCM (20 mL each). Combined organics were dried with MgSO₄, filtered, and concentrated in vacuo. Flash column chromatography on silica using a gradient of 2.5-25% MeOH in DCM was performed, affording separable BC-6 (301 mg, 78%) and BC-7 (40 mg, 10%) as white solids. 2′-O isomer (BC-6): ¹H NMR (600 MHz, methanol-d₄): δ 8.30 (s, 1H), 8.14 (s, 1H), 7.33-7.22 (m, 6H), 7.24-7.17 (m, 2H), 7.00-6.95 (m, 2H), 5.87 (d, J=9.3 Hz, 1H), 4.90 (dd, J=11.8, 7.5 Hz, 1H), 4.95-4.88 (m, 1H) 4.81 (dd, J=11.7, 8.5 Hz, 1H), 4.53-4.42 (m, 2H), 4.18 (dd, J=12.9, 2.1 Hz, 1H), 4.03 (t, J=9.2 Hz, 1H), 3.94 (dd, J=13.0, 1.2 Hz, 1H), 3.92 (t, J=9.0 Hz, 1H), 3.62 (dt, J=9.4, 1.8 Hz, 1H), 1.31-0.95 (m, 28H). 2′-O isomer (BC-6): ¹³C NMR (126 MHz, methanol-d₄): δ 157.4, 154.1, 151.1, 140.9, 137.0 (d, J=7.3 Hz), 136.5 (d, J=7.3 Hz), 129.6, 129.5, 129.0, 128.6, 120.0, 82.9, 81.0, 80.1, 76.7 (d, J=2.7 Hz), 70.9 (d, J=6.0 Hz), 70.6, 70.4 (d, J=5.9 Hz), 61.9, 18.0, 17.8, 17.7, 17.6, 14.8, 14.5, 14.1, 13.8. 3′-O isomer (BC-7): ¹H NMR (600 MHz, methanol-d₄): δ 8.31 (s, 1H), 8.22 (s, 1H), 7.38-7.30 (m, 10H), 5.65 (d, J=9.2 Hz, 1H), 5.14-5.07 (m, 2H), 5.07-5.00 (m, 2H), 4.55 (q, J=8.5 Hz, 1H), 4.43 (t, J=9.1 Hz, 1H), 4.18 (t, J=9.1 Hz, 1H), 4.15 (dd, J=12.8, 2.0 Hz, 1H), 3.94 (dd, J=12.8, 1.8 Hz, 1H), 3.59 (dt, J=9.4, 1.9 Hz, 1H), 1.14-0.86 (m, 28H). 3′-O isomer (BC-7): ¹³C NMR (126 MHz, methanol-d₄): δ 157.4, 154.0, 151.1, 141.1, 137.4 (d, J=7.0 Hz), 137.1 (d, J=7.2 Hz), 129.7, 129.6, 129.3, 120.1, 86.00, 85.4 (d, J=6.6 Hz), 85.0, 80.3, 72.2, 71.1 (d, J=5.6 Hz), 70.8 (d, J=5.3 Hz), 69.8 (d, J=5.4 Hz), 62.0, 18.0, 17.9, 17.8, 17.7, 17.5, 14.4, 14.3, 14.1.

Note: Some variability between experiments regarding amount of 1H-tetrazole and phosphoramidite needed. It is important to monitor conversion of sugar starting material to prevent bis-phosphorylation. Developed TLC plate (12:1 DCM: MeOH) was visualized using p-anisaldehyde stain, where 2′-O phosphate BC-6 stained brown and 3′-O phosphate BC-7 blue.

### Example 13. Step 3. Compounds BC-8 and BC-9

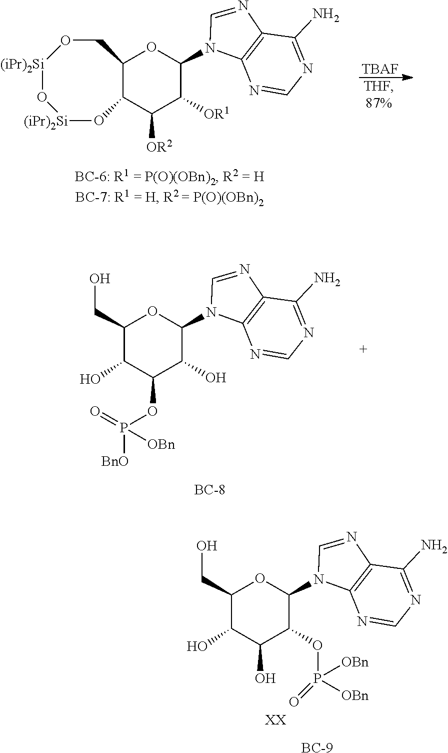

To a 10:1 solution of BC-6 and BC-7 (154 mg, 0.192 mmol, 1.00 equiv.), respectively, in THF (4 mL) at 0° C. was added TBAF (1M in THF, 480 μL, 0.48 mmol, 2.50 equiv.). After 15 min., AcOH (60 μL) was added, and the resulting solution was concentrated in vacuo. Flash column chromatography on silica using a gradient of 10-40% MeOH in DCM was performed, affording BC-8 (73 mg, 0.131 mmol, 68%) and BC-9 (20 mg, 0.036 mmol, 19%) which were able to be mostly separated after subsequent purification. 2′-O isomer (BC-9): ¹H NMR (500 MHz, methanol-d₄): δ 8.38 (s, 1H), 8.15 (s, 1H), 7.34-7.15 (m, 8H), 6.98-6.92 (m, 8H), 5.86 (d, J=9.3 Hz, 1H), 4.97-4.88 (m, 2H), 4.83 (dd, J=12.0, 8.5 Hz, 1H), 4.48 (dd, J=11.8, 6.8 Hz, 1H), 4.43 (dd, J=11.8, 8.5 Hz, 1H), 3.90 (dd, J=12.3, 1.5, 1H), 3.83 (t, J=8.8 Hz, 1H), 3.76 (dd, J=12.3, 5.0 Hz, 1H), 3.68-3.62 (m, 2H). 2′-O isomer (BC-9): ¹³C NMR (126 MHz, methanol-d₄): δ 157.4, 154.1, 150.9, 141.4, 137.0 (d, J=7.2 Hz), 136.6 (d, J=7.4 Hz) 129.5, 128.9, 128.5, 120.0, 82.8, 81.4, 79.7, 77.3 (d, J=2.6 Hz), 71.1, 70.9 (d, J=5.8 Hz), 70.3 (d, J=6.0 Hz), 62.2. 3′-O isomer (BC-8): ¹H NMR (500 MHz, methanol-d₄): δ 8.32 (s, 1H), 8.22 (s, 1H), 7.40-7.24 (m, 10H), 5.65 (d, J=9.3 Hz, 1H), 5.16 (d, J=7.5 Hz, 2H), 5.07-5.00 (m, 2H), 4.54 (q, J=8.8 Hz, 1H), 4.40 (t, J=9.2 Hz, 1H), 3.91 (dd, J=12.2, 2.1, 1H), 3.87 (t, J=9.0 Hz, 1H), 3.80 (dd, J=12.2, 5.1 Hz, 1H), 3.67 (ddd, J=9.8, 5.0, 2.1 Hz, 1H). 3′-O isomer (BC-8): ¹³C NMR (126 MHz, methanol-d₄): δ 157.4, 153.9, 150.8, 141.6, 137.5 (d, J=4.2 Hz), 137.4 (d, J=4.2 Hz), 129.6, 129.5, 129.0, 120.2, 85.9, 85.2, 80.7, 71.9 (d, J=3.2 Hz), 70.8 (d, J=5.8 Hz), 69.7 (d, J=3.2 Hz), 62.1. Note: Developed TLC plate (5:1 DCM: MeOH) was visualized using p-anisaldehyde stain, where 2′-O phosphate BC-9 stained brown and 3′-O phosphate BC-8 blue.

### Example 13. Step 4. N⁹—(β-glucopyranosyl)-(3′-O-phospho)-N¹-methyladenine (maglu #2, BC-10)

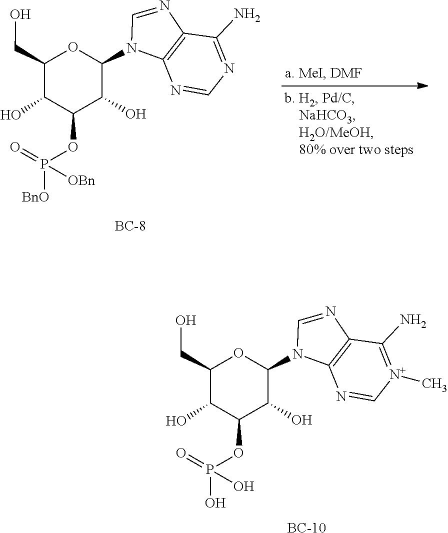 [SMILES: Nc1ncnc2c1ncn2[C@@H]1O[C@H](CO)[C@@H](O)[C@H](OP(=O)(OCc2ccccc2)OCc2ccccc2)[C@H]1O ; "BC-8" ; C[n+]1cnc2c(ncn2[C@@H]2O[C@H](CO)[C@@H](O)[C@H](OP(=O)(O)O)[C@H]2O)c1N ; "BC-10"]

A solution of BC-8 (12 mg, 0.021 mmol) and Mel (5.2 μL, 0.083 mmol, 3.97 equiv.) in DMF (1 mL) was stirred for 24 hr at 40° C. The resulting yellow solution was then dissolved in 1.5 mL MeOH/1.0 mL H₂O. NaHCO₃ (7.5 mg, 0.089 mmol, 4.25 equiv.) and Pd/C (12 mg) were added, the suspension was sparged with Ar for 5 min. then switched to H₂, through which the suspension was rapidly stirred under for 2 hr. After sparging with Ar, AcOH (20 μL) was added and the reaction mixture was filtered through celite. The collected filtrate was concentrated to dryness in vacuo. Flash column chromatography on C18 using 100% H₂O (w/0.1% formic acid) afforded maglu #2 (BC-10, 6.8 mg, 80% over two steps) as a white solid. maglu #2 was compared to the corresponding peak in C. elegans wildtype (N2) endo-metabolome samples by HILIC-HRMS (Method D) and MS². HRMS (ESI) m/z: [M+H]⁺ calcd for C₁₂H₁₉N₅O₈P⁺ 392.0966; found 392.0953. Note: Addition of NaHCO₃ was required as non-basified solutions led to only mono-debenzylation. AcOH was added to prevent any partial Dimroth rearrangement of samples during the concentration step as well during evaluation of sample purity w/crude NMR.

### Example 14. Step 1. Compound BC-11

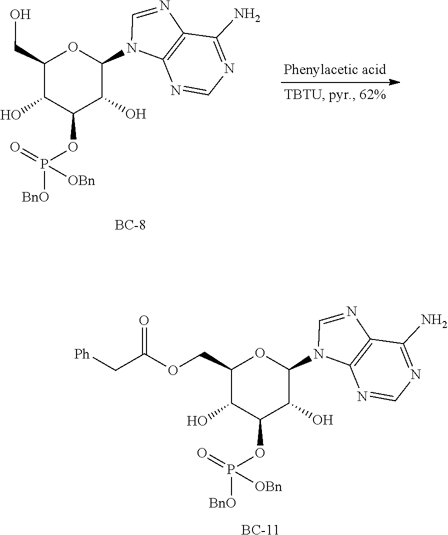

Phenylacetic acid (19 mg, 0.141 mmol, 2.82 equiv.) and TBTU (45 mg, 0.141 mmol, 2.82 equiv.) were added to a solution of BC-8 (28 mg, 0.050 mmol, 1.00 equiv.) in 0.7 mL dry pyridine. The resulting mixture was stirred for 3 hr at room temp, and then concentrated to dryness in vacuo. Flash column chromatography on silica using a gradient of 2.5-30% MeOH in DCM was performed, affording BC-11 (21 mg, 62%) as a colorless oil, with some fractions containing co-eluting HOBt. ¹H NMR (600 MHz, methanol-d₄): δ 8.21 (s, 1H), 8.15 (s, 1H), 7.38-7.26 (m, 10H), 7.22-7.12 (m, 5H), 5.63 (d, J=9.3 Hz, 1H), 5.17-5.07 (m, 4H), 4.58-4.49 (m, 2H), 4.35 (t, J=9.2 Hz, 1H), 4.32 (dd, J=12.4, 5.9 Hz), 3.88 (ddd, J=10.0, 5.8, 2.2 Hz, 1H), 3.79 (t, J=9.5 Hz, 1H), 3.63 (dd, J=15.0 Hz, 1H), 3.59 (d, J=15.0 Hz, 1H). ¹³C NMR (126 MHz, methanol-d₄): δ 173.2, 157.5, 154.0, 151.0, 150.1, 141.3, 137.5 (d, J=2.8 Hz, 1H), 137.4 (d, J=2.8 Hz, 1H), 135.6, 130.4, 129.6, 129.5, 129.4, 129.0, 128.0, 125.6, 120.3, 85.6 (d, J=6.7 Hz, 1H), 84.8, 77.9, 71.9 (d, J=3.8 Hz, 1H), 70.8 (d, J=5.8 Hz, 1H), 70.1 (d, J=3.8 Hz, 1H), 64.4, 41.8.

### Example 14. Step 2. maglu #11 (BC-12)

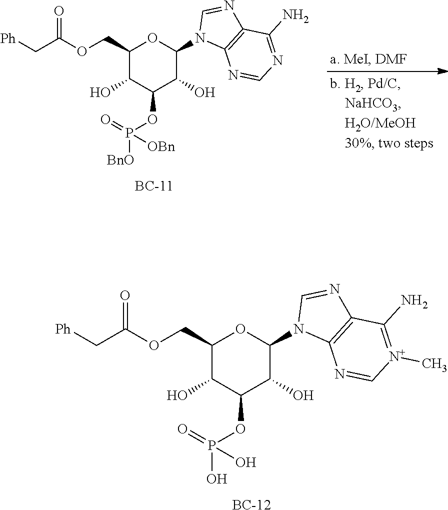

A solution of BC-11 (6.0 mg, 8.88 μmol, 1.00 equiv.) and Mel (2.2 μL, 35.6 μmol, 4.01 equiv.) in dry DMF (0.3 mL) was stirred for 24 hr at 40° C. The resulting yellow solution (a 2:1 mixture of mono and bis-benzylated products, respectively) was concentrated in vacuo and then dissolved in 0.5 mL MeOH and 75 μL H₂O. To the solution was added NaHCO₃ (2.5 mg, 30.0 μmol, 3.38 equiv.) in 22.5 μL H₂O and Pd/C (17 mg, 10% w/w). The suspension was sparged with Ar for 5 min. then switched to H₂, through which the suspension was rapidly stirred under for 1.5 hr. After sparging with Ar for 5 min., AcOH (30 μL) was added and the reaction mixture was then filtered through celite using MeOH/H₂O. The collected filtrate was concentrated to dryness in vacuo. Purification by preparative HPLC (see Methods) afforded maglu #11 (BC-12, 1.4 mg, 30% over two steps) as a white solid. maglu #3 was found to be identical to the corresponding peak on C18 in C. elegans wildtype (N2), fem-3 (gf), him-5 endo-metabolome samples by HPLC-HRMS (Method A) and MS². Bis-benzylated and mono-benzylated species-HRMS (ESI) m/z: [M+H]⁺ calcd for C₃₄H₃₇N₅O₉P⁺ 690.2323 found 690.2310 and [M+H]⁺ calcd for C₂₇H₃₁N₅O₉P⁺ 600.1854 found 600.1840 for bis-benzylated and mono-benzylated species, respectively. maglu #3 (BC-12)—HRMS (ESI) m/z: [M+H]′ calcd for C₂₀H₂₅N₅O₉P⁺ 510.1384; found 510.1398. A bis-methylated species (c.a. 25%) was also observed. [M+H]⁺ calcd for C₂₁H₂₇N₅O₉P⁺ 524.1541; found 524.1556. This impurity was removed via preparative HPLC (see Methods).

### Synthesis of Glucosyl Guanine Derivatives

### Example 15. Step 1. Compound BC-14

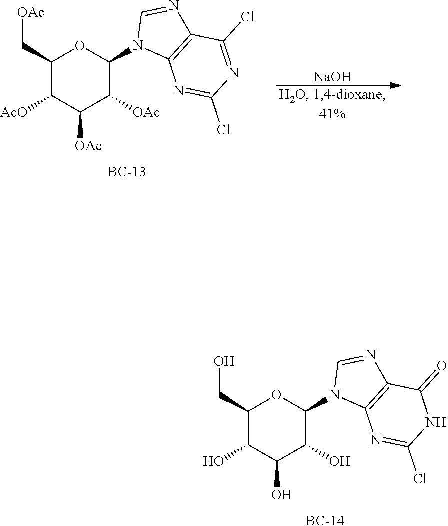

Compound BC-14 was synthesized according to a previously reported procedure. A suspension containing BC-13 (2.1 g, 4.05 mmol, 1.00 equiv.), NaOH (1.62 g, 40.5 mmol, 10 equiv.), H₂O (50 mL), and 1,4-dioxane (20 mL) was heated at 100° C. for 4 hr. The resulting dark red solution was neutralized by the addition of AcOH and then concentrated to dryness in vacuo. Flash column chromatography on C18 using a gradient of 0-50% ACN in H₂O (w/0.1% AcOH) afforded BC-14 (560 mg, 41%) as a yellow solid. ¹H NMR (600 MHz, DMSO-d₆): δ 8.30 (s, 1H), 5.37 (d, J=4.1 Hz, 1H), 5.30 (d, J=9.3 Hz, 1H), 5.30-5.23 (m, 1H), 5.20-5.08 (m, 1H), 4.65-4.51 (m, 1H), 3.89-3.83 (m, 1H), 3.70 (d, J=11.4 Hz, 1H), 3.46-3.34 (m, 3H), 3.24 (t, J=9.1 Hz, 1H). (MODERNA THERAPEUTICS INC—WO2017/66793, 2017, A1)

### Example 15. Step 2. N⁹—(β-glucopyranosyl)-N²-methylguanine (BC-15, mgglu #3)

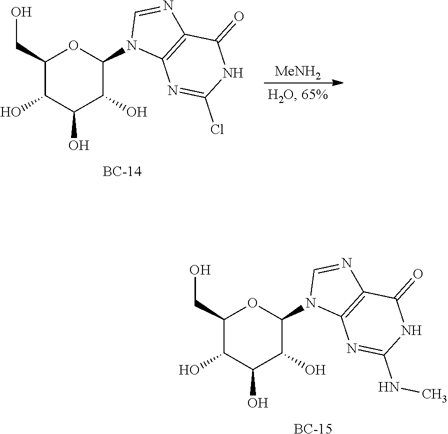

A solution containing BC-14 (330 mg, 0.99 mmol, 1.00 equiv.) and MeNH₂ (12 mL, 40% in H₂O, 154 mmol, 156 equiv.) was heated at 100° C. in a sealed container for 15 hr. The resulting solution was acidified to pH~5 w/AcOH, transferred to a round-bottom flask, and then concentrated to dryness in vacuo. Flash column chromatography on C18 using a gradient of 0%-30% ACN in H₂O (w/0.1% AcOH) afforded BC-15 (mgglu #3, 210 mg, 65%) as an off-white solid. mgglu #3 was found to be identical to the corresponding peak using HILIC-MS (Method C) in C. elegans wildtype (N2) samples by HPLC-HRMS. ¹H NMR (600 MHz, DMSO-d₆): δ 10.67 (br s, 1H), 7.83 (s, 1H), 6.36-6.32 (m, 1H), 5.32 (d, J=4.8 Hz, 1H), 5.26-5.19 (m, 1H), 5.20 (d, J=9.2 Hz, 1H), 5.08 (d, J=4.3 Hz, 1H), 4.59 (t, J=5.8 Hz, 1H), 3.83 (td, J=9.3, 3.8 Hz, 1H), 3.70 (dd, J=11.9, 4.1 Hz, 1H), 3.46-3.40 (m, 1H), 3.39-3.31 (m, 2H), 3.23-3.18 (m, 1H), 2.82 (d, J=4.7 Hz, 3H). 13C (126 MHz, DMSO-d₆): δ 157.0, 153.4, 151.3, 135.7, 116.3, 82.2, 80.0, 77.3, 71.4, 69.8, 61.0, 27.5.

HRMS (ESI) m/z: [M+Na]⁺ calcd for C₁₂H₁₇N₅O₆Na⁺350.1071; found 350.1053.

### Example 16. Step 1. N⁹—(β-glucopyranosyl)-N²,N²-dimethylguanine (BC-16, dmgglu #3)

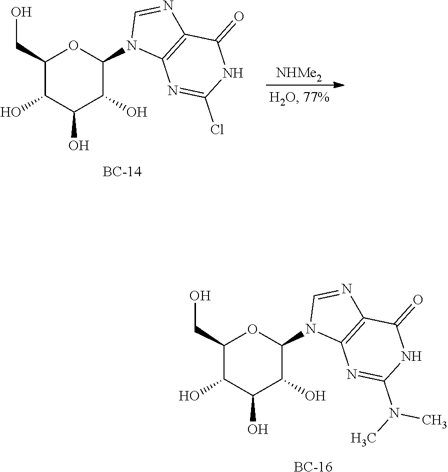

To BC-14 (60 mg, 0.181 mmol, 1.00 equiv.) in a high-pressure flask was added 40% NHMe₂ in H₂O (2.5 mL). The flask was sealed and heated to 100° C., at which the resulting solution was stirred for 14 hr. The solution was transferred to a round-bottom flask and concentrated to dryness in vacuo. Flash column chromatography on C18 using a gradient of 0-100% ACN (w/0.1% formic acid) in H₂O (w/0.1% formic acid), followed by additional purification with flash column chromatography on silica using a gradient of 20-60% MeOH in DCM afforded BC-16 (48 mg, 77%) as a white solid. dmgglu #3 (BC-16) was compared to isomer peaks using HILIC-MS (Method C) in C. elegans and C. briggsae wildtype samples by HILIC-HRMS. ¹H NMR (500 MHz, DMSO-d₆): δ 10.68 (br s, 1H), 7.85 (s, 1H), 5.53-5.23 (m, 2H), 5.20 (d, J=9.2 Hz, 1H), 5.18 (br s, 1H), 4.61 (br s, 1H), 3.85 (t, J=9.1 Hz, 1H), 3.69 (d, J=12.0, 1H), 3.42 (dd, J=12.0, 6.1 Hz, 1H), 3.36-3.30 (m, 3H), 3.20 (t, J=9.2 Hz, 1H), 3.07 (s, 6H). ¹³C NMR (126 MHz, DMSO-d₆): 157.5, 153.0, 151.1, 136.3, 115.6, 82.3, 79.9, 77.2, 71.3, 69.8, 60.9, 37.6. HRMS (ESI) m/z: [M+Na]⁺ calcd for C₁₃H₁₉N₅O₆Na⁺364.1227; found 364.1218.

### Example 17. Step 1. Compound BC-18

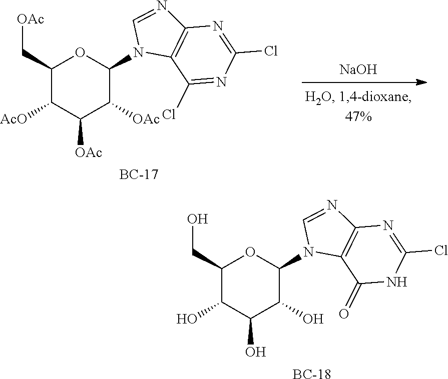

A solution containing BC-17 (515 mg, 0.99 mmol, 1.00 equiv.), NaOH (400 mg, 10.0 mmol, 10.10 equiv.), H₂O (13 mL) and 1,4-dioxane (5 mL) was heated at 100° C. for 4 hr. The dark red solution was cooled to 0° C., acidified to pH=4 by the addition of AcOH, and concentrated to dryness in vacuo. Flash column chromatography on C18 using a gradient of 0%-50% ACN in H₂O (w/0.1% formic acid) afforded BC-18 (157 mg, 47%) as a light brown solid. ¹H NMR (600 MHz, DMSO-d₆): δ 8.47 (s, 1H), 5.68 (d. J=9.4 Hz, 1H), 5.38 (br s, 1H), 5.28 (br s, 1H), 5.11 (br s, 1H), 4.58-4.50 (m, 1H), 3.84 (d, J=9.2 Hz, 1H), 3.69 (d, J=11.7 Hz, 1H), 3.47-3.42 (m, 1H), 3.37-3.30 (m, 3H), 3.26 (dd, J=9.7, 3.5 Hz, 1H). ¹³C NMR (126 MHz, DMSO-d₆): 163.1, 156.7, 154.5, 143.4, 113.8, 85.0, 80.1, 77.0, 72.0, 69.5, 60.9. HRMS (ESI) m/z: [M+Na]⁺ calcd for C₁₁H₁₃ClN₄O₆Na⁺ 355.0416; found 355.0410. The compound was primarily detected as its in-source fragment: [M+H]⁻ calcd for C₅H₄ClN₄O⁺ 171.0068; found 171.0066.

### Example 17. Step 2. N⁷—(β-glucopyranosyl)-N²,N²-dimethylguanine, dmgglu #1 (BC-19)

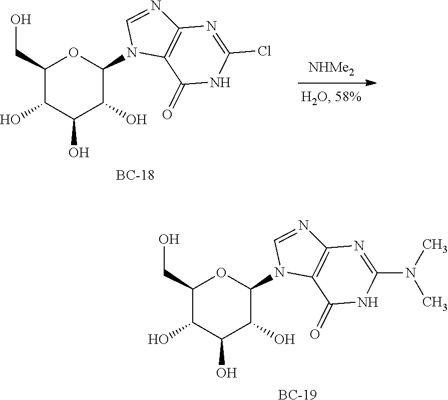

To BC-18 (51 mg, 0.154 mmol, 1.00 equiv.) in a high-pressure flask was added 40% NHMe₂ in H₂O (5.1 mL). The flask was sealed and heated to 100° C., at which the resulting solution was stirred for 19 hr. The solution was cooled to room temp., transferred to a round-bottom flask, neutralized with AcOH, and concentrated to dryness in vacuo. Flash column chromatography on C18 using a gradient of 0-100% ACN in H₂O (w/0.1% formic acid) afforded dmgglu #1 (BC-19, 30 mg, 58%) as an off-white solid. dmgglu #3 was found to be identical to the corresponding peak using HILIC-MS (Method C) in C. elegans wildtype (N2) samples by HPLC-HRMS. ¹H NMR (500 MHz, DMSO-d₆): 10.84 (br s, 1H), 8.20 (s, 1H), 5.57 (d, J=9.3 Hz, 1H), 5.34 (br s, 1H), 5.25 (br s, 1H), 5.09 (br s, 1H), 4.53 (t, J=6.1 Hz, 1H), 3.84 (t, J=9.2 Hz, 1H), 3.68 (d, J=12.0 Hz, 1H), 3.47-3.28 (m, 3H), 3.24 (t, J=9.3 Hz, 1H), 3.03 (s, 6H). ¹³C NMR (126 MHz, DMSO-d₆): δ 159.5, 154.9, 152.7, 142.3, 107.5, 84.7, 79.8, 77.2, 71.8, 69.5, 60.9, 37.9. HRMS (ESI) m/z: [M+Na]⁺ calcd for C₁₃H₁₉N₅O₆Na⁺ 364.1227; found 364.1217. A large fraction of the sample was detected as the in-source fragment: [M+H]⁺ calcd for C₇H₁₀N₅O⁺ 180.0880; found 180.0877.

### Example 18. Step 1. mgglu #1 (BC-21) and mgglu #5 (BC-22)

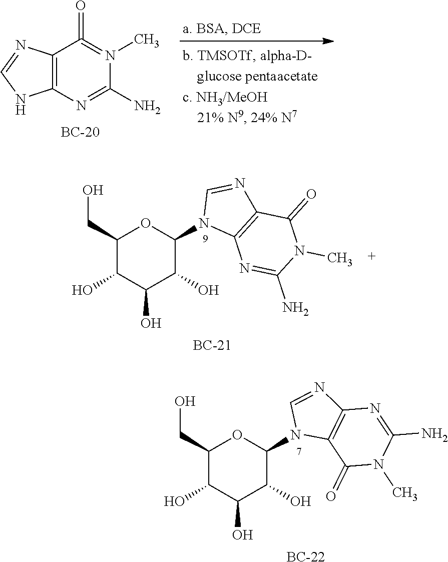

Under Ar, a suspension of N¹-methylguanine (BC-20, 750 mg, 4.54 mmol, 1.36 equiv.), N,O-bis(trimethylsilyl) acetamide (2.4 mL, 9.84 mmol, 3.02 equiv.) and DCE (20 mL) was refluxed for 1 hr until the solution was homogeneous. After cooling to room temp., TMSOTf (1.35 mL, 7.48 mmol, 2.29 equiv.) and alpha-D-glucose pentaacetate (1.27 g, 3.26 mmol, 1.00 equiv.) were added and the resulting solution was refluxed for 36 hr. The resulting orange solution was then concentrated to dryness in vacuo, followed by the addition of NH₃/MeOH (7N, 38 mL, 266 mmol, 81 equiv.). The resulting solution was stirred for 7 hr at room temp, and concentrated to dryness in vacuo. Flash column chromatography on C18 using a gradient of 0%-100% ACN in H₂O (w/0.1% AcOH) was first performed, of which fractions containing products BC-21 and BC-22 and 1-methylguanine (BC-20) were collected (elution at ~10% ACN). The dried mixture was dissolved in MeOH and filtered, followed by concentration in vacuo with 15 g of silica gel for dry-loading. Flash column chromatography on silica using a gradient of 35%-100% MeOH in DCM was then performed, which afforded BC-22 (256 mg, 24%) and BC-21 (219 mg, 21%), of which could be mostly separated with subsequent chromatography. mgglu #1 (BC-21) and mgglu #5 (BC-22) were compared to isomer peaks (m/z=350.1071) using HILIC-MS in C. elegans wildtype (N₂) samples by HILIC-HRMS (Method C) and MS². ¹H NMR (N⁹-isomer, BC-21) (600 MHz, D₂O): δ 7.98 (s, 1H), 5.47 (d, J=9.4 Hz, 1H), 4.16 (t, J=9.0 Hz, 1H), 3.93 (dd, J=12.4, 1.7 Hz, 1H), 3.82 (dd, J=12.3, 5.0 Hz, 1H), 3.74 (dd, J=9.7, 1.8 Hz, 1H), 3.71 (t, J=8.9 Hz, 1H), 3.66 (t, J=9.3 Hz, 1H), 3.41 (s, 3H). ¹³C NMR (N⁹-isomer, BC-21) (126 MHz, D₂O): δ 159.2, 155.4, 150.1, 138.6, 116.4, 83.4, 79.3, 76.9, 71.8, 69.6, 61.0, 29.2. HRMS (ESI) m/z: [M+Na]⁺ calcd for C₁₂H₁₇N₅O₆Na⁺ 350.1071; found 350.1060. ¹H NMR (N⁷-isomer, BC-22) (600 MHz, D₂O): δ 8.29 (s, 1H), 5.79 (d, J=9.2 Hz, 1H), 4.18 (t, J=9.0 Hz, 1H), 3.94 (dd, J=12.4, 1.7 Hz, 1H), 3.80 (dd, J=12.4, 5.5 Hz, 1H), 3.74 (dd, J=9.5, 1.7 Hz, 1H), 3.71 (t, J=8.9 Hz, 1H), 3.66 (t, J=9.3 Hz, 1H), 3.47 (s, 3H). ¹³C NMR (N⁷-isomer, BC-22) (126 MHz, D₂O): δ 158.1, 155.9, 155.2, 144.6, 108.2, 85.7, 79.3, 76.8, 72.7, 69.7, 61.2, 29.1. HRMS (ESI) m/z: [M+Na]⁺ calcd for C₁₂H₁₇N₅O₆Na⁺ 350.1071; found 350.1053.

### Example 18. Step 2. 6′-O, 4′-O-TIPDSi-N⁷—(β-glucopyranosyl)-N¹-methylguanine (BC-23)

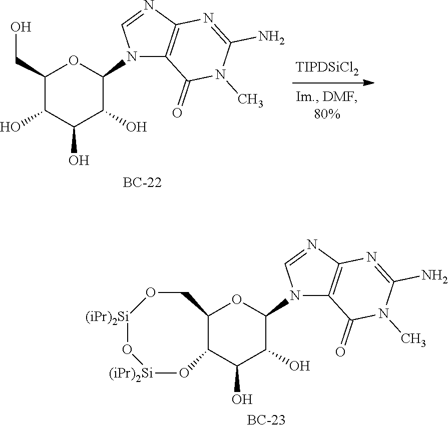

To a solution of BC-22 (120 mg, 0.367 mmol, 1.00 equiv.) in DMF (2 mL) at 0° C. was added TIPDSiCl₂ (175 μL, 0.504 mmol, 1.50 equiv.) and imidazole (104 mg, 1.53 mmol, 4.55 equiv.). The reaction mixture was stirred for 15 min at 0° C. and then diluted with DCM, followed by addition of H₂O. Organics were extracted 4× with DCM, combined, and then basified using sat. aq. NaHCO₃. Organics were then extracted from the aq. layer 3× with DCM, dried using MgSO₄, filtered, and concentrated in vacuo. Flash column chromatography on silica using a gradient of 5-50% MeOH in DCM was performed, affording BC-23 (168 mg, 80%) as a white solid. ¹H NMR (500 MHz, methanol-d₄): δ 8.17 (s, 1H), 5.79 (d, J=9.4 Hz, 1H), 4.16 (dd, J=12.8, 2.1 Hz, 1H), 3.99 (t, J=9.0 Hz, 1H), 3.95 (t, J=9.0 Hz, 1H), 3.90 (dd, J=12.8, 1.0 Hz, 1H), 3.60 (t, J=9.0 Hz, 1H), 3.49 (dt, J=9.5, 1.6 Hz, 1H), 3.45 (s, 3H), 1.25-0.96 (m, 28H). ¹³C NMR (126 MHz, methanol-d₄): δ 158.8, 155.8, 143.3, 136.3, 109.1, 86.7, 80.7, 78.4, 74.0, 70.4, 62.1, 28.7, 18.0, 17.8, 17.7, 17.6, 14.9, 14.5, 14.0, 13.8.

### Example 18. Step 3. Compounds BC-24 and BC-25

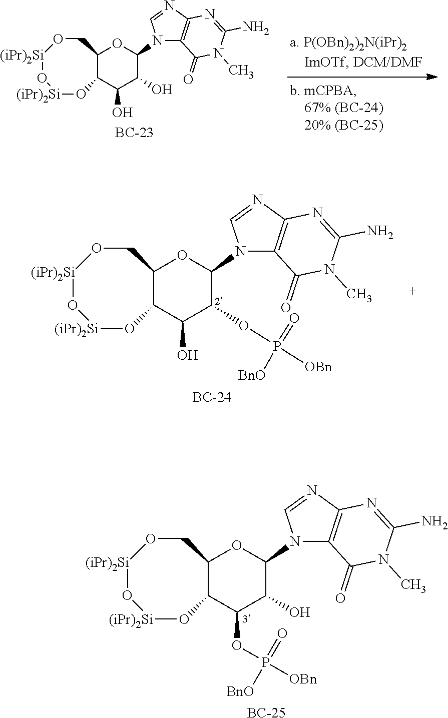

To BC-23 (153 mg, 0.27 mmol, 1.00 equiv.) in DCM (2 mL) and DMF (1 mL) was added dibenzyl N,N-diisopropylphosphoramidite (0.36 mL, 1.07 mmol, 3.96 equiv.), and ImOTf (277 mg, 1.27 mmol, 4.70 equiv.) incrementally over a 2 hr period. Note: this was done to ensure minimization of bis-phosphitylation products. The reaction mixture was then cooled to −78° C. after which mCPBA (77% max, 165 mg, 0.74 mmol, 2.73 equiv.) was added. The resulting mixture was stirred at −78° C. for 20 min, and was then quenched with the addition of sat. aq. NaHCO₃ (10 mL) followed by addition of DCM (20 mL). The organic layer was collected and the aqueous layer was extracted an additional 2× with DCM (20 mL each). Combined organics were dried with MgSO₄, filtered, and concentrated in vacuo. Flash column chromatography on silica using a gradient of 5-40% MeOH in DCM was performed, affording separable BC-24 (149 mg, 67%) and BC-25 (44 mg, 20%) as white solids. 2′-O isomer (BC-24): ¹H NMR (500 MHz, methanol-d₄): δ 8.21 (br s, 1H), 7.36-7.13 (m, 8H), 7.14-6.97 (m, 2H), 6.24-5.63 (m, 1H), 5.04-4.89 (m, 2H), 4.78-4.50 (m, 3H), 4.16 (d, J=12.6 Hz, 1H), 4.11-3.95 (m, 1H), 3.91 (d, J=12.6 Hz, 1H), 3.88-3.77 (m, 1H), 3.59-3.47 (m, 1H), 3.25 (br s, 3H), 1.30-0.93 (m, 28H). 2′-O isomer (BC-24): ¹³C NMR (126 MHz, methanol-d₄): δ 156.8, 137.1, 136.8, 129.5, 129.4, 128.9, 109.1, 80.8, 76.9, 70.9, 70.4, 61.9, 28.6, 18.1, 17.8, 17.7, 17.6, 14.8, 14.5, 14.0, 13.8. 3′-O isomer (BC-25): ¹H NMR (500 MHz, methanol-d₄): δ 8.19 (s, 1H), 7.39-7.28 (m, 10H), 5.77 (d, J=8.5 Hz, 1H), 5.16-5.06 (m, 2H), 5.08-4.96 (m, 2H), 4.54-4.40 (m, 2H), 4.20 (t, J=9.0 Hz, 1H), 4.14 (dd, J=12.8, 1.4 Hz, 1H), 3.93 (dd, J=12.7, 1.7 Hz, 1H), 3.52 (d, J=9.2 Hz, 1H), 3.46 (s, 3H), 1.19-0.82 (m, 28H). 3′-O isomer (BC-25): ¹³C NMR (126 MHz, methanol-d₄): δ 159.0, 155.9, 155.6, 144.0, 137.4 (J=7.0 Hz), 137.2 (d, J=7.4 Hz), 129.7, 129.6, 129.3, 129.0, 109.0, 87.0, 85.6 (d, J=6.8 Hz), 80.1, 72.7, 71.1 (d, J=5.6 Hz), 70.8 (d, J=5.3 Hz), 69.6 (d, J=5.6 Hz), 62.0, 28.7, 18.0, 17.9, 17.8, 17.7, 17.5, 14.4, 14.3, 14.2, 14.1. Note: 2′-O dibenzyl phosphate isomer (BC-24) exhibited extreme line broadening for several signals.

### Example 18. Step 4. Compounds BC-26 and BC-27

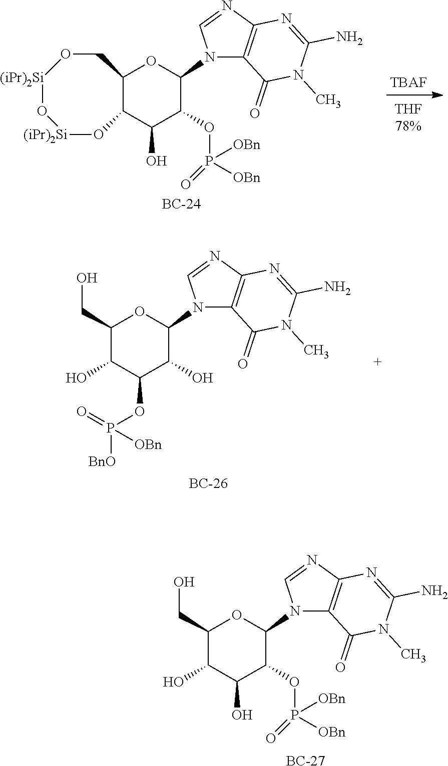

To a solution of BC-24 (90 mg, 0.108 mmol, 1.00 equiv.) in THF (3 mL) at 0° C. was added TBAF (IM in THF, 275 μL, 0.27 mmol, 2.50 equiv.). After 15 min., AcOH (75 μL) was added, and the resulting solution was concentrated in vacuo. Flash column chromatography on silica using a gradient of 5-40% MeOH in DCM was performed, affording BC-26 (37.5 mg, 0.036 mmol, 59%) and BC-27 (11.5 mg, 8 mmol, 18%) of which were mostly separable. 3′-O isomer (BC-26): ¹H NMR (500 MHz, methanol-d₄): δ 8.23 (s, 1H), 7.39-7.21 (m, 10H), 5.78 (d, J=9.1 Hz, 1H), 5.15 (d, J=7.5 Hz, 2H), 5.13-5.10 (m, 2H), 4.49 (td, J=9.1, 8.1 Hz, 1H), 4.36 (t, J=9.2 Hz, 1H), 3.90 (dd, J=12.2, 2.3 Hz, 1H), 3.84 (t, J=9.5 Hz, 1H), 3.77 (dd, J=12.2, 5.2 Hz, 1H), 3.63 (ddd, J=9.9, 5.2, 2.3 Hz, 1H), 3.47 (s, 3H). 3′-O isomer (BC-26): ¹³C NMR (126 MHz, methanol-d₄): δ 159.3, 155.9, 155.8, 144.4, 137.5 (d, J=3.5 Hz), 137.4 (d, J=3.5 Hz), 129.6, 129.5, 129.1, 129.0, 109.0, 86.8, 86.0 (d, J=6.9 Hz), 80.5, 72.7 (d, J=3.5 Hz), 70.8 (d, J=5.8 Hz), 69.7 (d, J=3.2 Hz), 62.2, 28.8. 2′-O isomer (BC-27): ¹H NMR (600 MHz, methanol-d₄): δ 8.28 (s, 1H), 7.40-6.97 (m, 10H), 6.17-5.86 (m, 1H), 5.00-4.93 (m, 1H), 4.67 (dd, J=12.3, 6.7 Hz, 1H), 4.65-4.52 (m, 1H), 3.89 (dd, J=12.2, 1.9 Hz, 1H), 3.78 (t, J=9.0 Hz, 1H), 3.73 (dd, J=12.2, 5.6 Hz, 1H), 3.64 (t, J=9.5 Hz, 1H), 3.63 (ddd, J=9.6, 5.6, 2.0 Hz, 1H), 3.27 (s, 3H).

### Example 18. Step 5. Compound BC-26

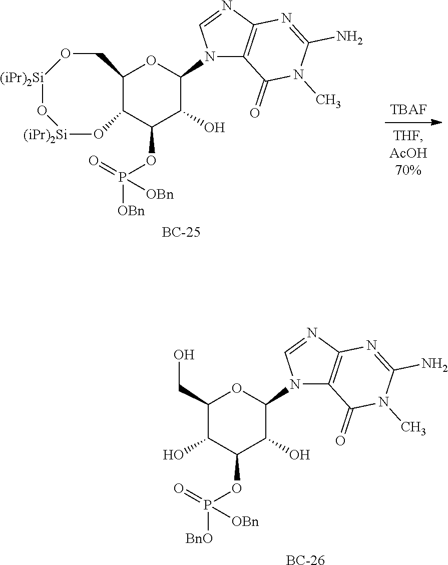

To a solution of BC-25 (65 mg, 0.078 mmol, 1.00 equiv.) in THF (3 mL) containing AcOH (20 μL) at 0° C. was added TBAF (IM in THF, 200 μL, 0.20 mmol, 2.56 equiv.). The solution was slowly warmed to RT over a 4 hr period, then additional AcOH (40 μL) was added, and the reaction mixture was concentrated in vacuo. Flash column chromatography on silica using a gradient of 15-50% MeOH in DCM was performed, afforded BC-26 (32 mg, 70%).

### Example 18. Step 6. mgglu #6 (BC-28)

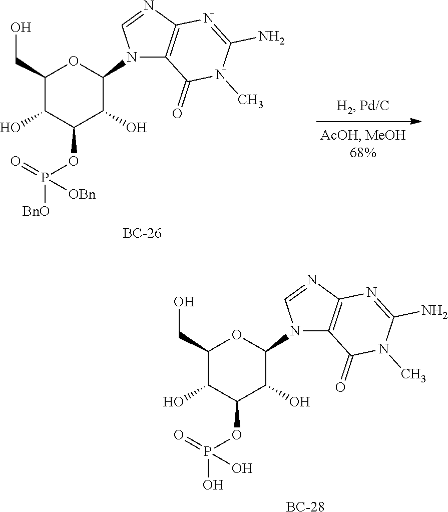 [SMILES: Cn1c(N)nc2ncn([C@@H]3O[C@H](CO)[C@@H](O)[C@H](OP(=O)(OCc4ccccc4)OCc4ccccc4)[C@H]3O)c2c1=O ; "BC-26" ; Cn1c(N)nc2ncn([C@@H]3O[C@H](CO)[C@@H](O)[C@H](OP(=O)(O)O)[C@H]3O)c2c1=O ; "BC-28"]

A suspension containing BC-26 (17 mg, 0.020 mmol, 1.00 equiv.), Pd/C (35 mg, 10% w/w), AcOH (300 μL) and MeOH (3 mL) was sparged with Ar for 5 min. then switched to H₂, through which the suspension was rapidly stirred under for 4 hr. After sparging with Ar, the reaction mixture was filtered through celite, washed with MeOH/H₂O, and the collected filtrate was concentrated to dryness in vacuo affording mgglu #6 (BC-28, 8 mg, 68%) at 92% purity. mgglu #6 was found to be identical to the major isomer peak in C. elegans wildtype (N₂) endo-metabolome samples by HILIC-HRMS. Chromatographic Method E was used. mgglu #6-HRMS (ESI) m/z: [M+H]⁺ calcd for C₁₂H₁₉N₅O₉P⁻ 408.0915; found 408.0914.

### Example 19. Step 1. Compound BC-29

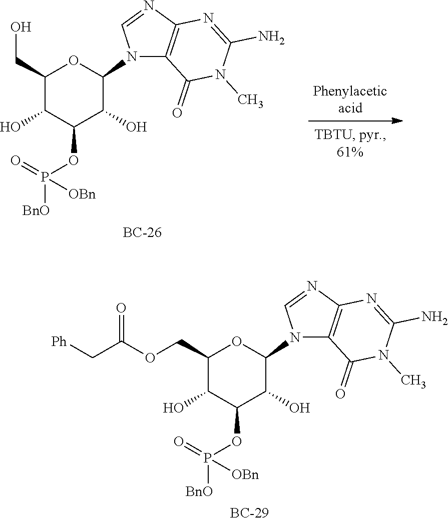

Phenylacetic acid (8 mg, 0.059 mmol, 4.21 equiv.) and TBTU (19 mg, 0.059 mmol, 4.21 equiv.) were added to a solution of BC-26 (8.2 mg, 0.014 mmol, 1.00 equiv.) in 1 mL dry pyridine. The resulting mixture was stirred for 4 hr at room temp., MeOH (1 mL) was added, transferred to a round-bottom flask, and then concentrated to dryness in vacuo. Flash column chromatography on silica using a gradient of 2.5-40% MeOH in DCM was performed, affording BC-29 (6.0 mg, 61%) as a white solid. Note: co-eluting HOBt was separated by subsequent chromatography. ¹H NMR (500 MHz, methanol-d₄): δ 8.10 (s, 1H), 7.40-7.15 (m, 15H), 5.73 (d, J=9.1 Hz, 1H), 5.17-5.08 (m, 4H), 4.54 (dd, J=12.1, 1.8 Hz, 1H), 4.47 (q, J=8.7 Hz, 1H), 4.37 (t, J=9.1 Hz, 1H), 4.29 (dd, J=12.1, 4.9 Hz, 1H), 3.84-3.76 (m, 2H), 3.67 (d, J=15.3 Hz, 1H), 3.63 (d, J=15.3 Hz, 1H), 3.44 (s, 3H). ¹³C NMR (126 MHz, methanol-d₄): δ 173.2, 159.3, 155.9, 155.7, 144.2, 137.5 (d, J=2.6 Hz), 137.4 (d, J=2.5 Hz), 135.6, 130.3, 129.6, 129.5, 129.4, 129.0, 128.0, 108.9, 86.7, 85.7, 85.6 (d, J=7.0 Hz), 77.7, 72.4 (d, J=3.2 Hz), 70.8 (d, J=5.9 Hz), 70.0 (d, J=3.4 Hz), 64.4, 41.8, 28.8.

### Example 19. Step 2. mgglu #51 (BC-30)

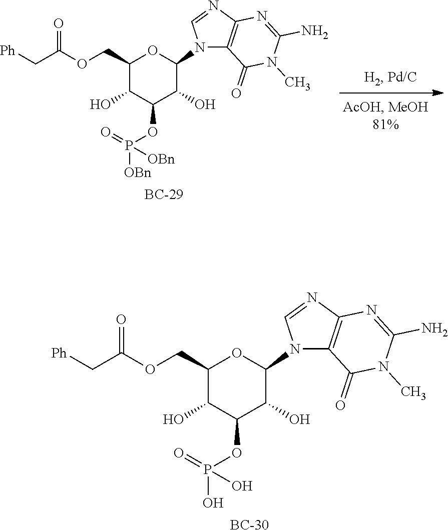

A suspension containing BC-29 (5 mg, 0.0071 mmol, 1.00 equiv.), Pd/C (6.6 mg, 10% w/w), AcOH (47 μL) and MeOH (2 mL) was sparged with Ar for 5 min. then switched to H₂, through which the suspension was rapidly stirred under for 1.5 hr. After sparging with Ar, the reaction mixture was filtered through celite using MeOH/H₂O and the collected filtrate was concentrated to dryness in vacuo, affording mgglu #51 (BC-30, 3.0 mg, 81%) which was deemed pure enough for no further purification steps. mgglu #51 was found to be identical to the major isomer peak on C18 in C. elegans wildtype (N2), fem-3 (gf), and him-5 endo-metabolome samples by HPLC-HRMS (Method B) and MS². mgglu #51-HRMS (ESI) m/z: [M+H]⁺ calcd for C₂₀H₂₅N₅O₁₀P⁺526.1333; found 526.1332.

### Example 20. Step 1. Compound BC-31

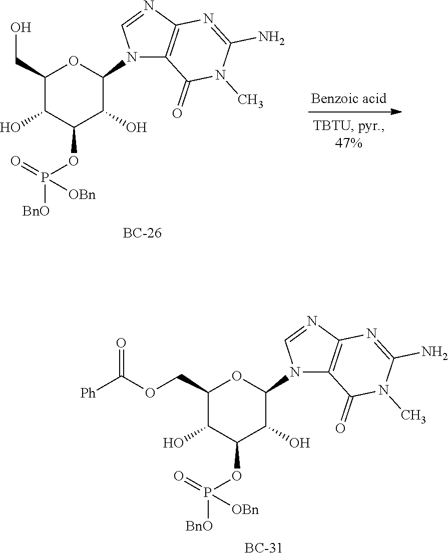

Benzoic acid (22 mg, 0.18 mmol, 9.49 equiv.) and TBTU (55 mg, 0.14 mmol, 7.64 equiv.) were added to a solution of BC-26 (11 mg, 0.019 mmol, 1.00 equiv.) in 1.5 mL dry pyridine. The resulting mixture was stirred for 10 hr at room temp., MeOH (2 mL) was added, transferred to a round-bottom flask, then concentrated to dryness in vacuo. Flash column chromatography on silica using a gradient of 5-40% MeOH in DCM was performed, affording BC-31 (6.0 mg, 47%) as a white solid. Note: co-eluting HOBt was separated by subsequent chromatography. ¹H NMR (500 MHz, methanol-d₄): δ 8.19 (s, 1H), 8.06 (d, J=7.2 Hz, 2H), 7.60 (t, J=7.4 Hz, 1H), 7.47 (t, J=7.8 Hz, 2H), 7.39-7.19 (m, 10H), 5.79-5.69 (m, 1H), 5.16 (d, J=7.4 Hz, 2H), 5.14-5.10 (m, 2H), 4.70 (dd, J=12.1, 1.7 Hz, 1H), 4.62-4.46 (m, 3H), 4.03-3.91 (m, 2H), 3.40 (s, 3H). ¹³C NMR (126 MHz, methanol-d₄): δ 167.8, 159.4, 155.9, 155.5, 144.4, 137.5 (d, J=3.1 Hz), 137.4 (d, J=3.3 Hz), 134.3, 131.3, 130.8, 129.6, 129.50, 129.4, 129.0, 108.9, 87.1, 85.7 (d, J=7.1 Hz), 77.8, 72.2 (d, J=3.5 Hz), 70.8 (d, J=6.0 Hz), 69.9 (d, J=3.4 Hz), 64.6, 28.8.

### Example 20. Step 2. mgglu #52 (BC-32)

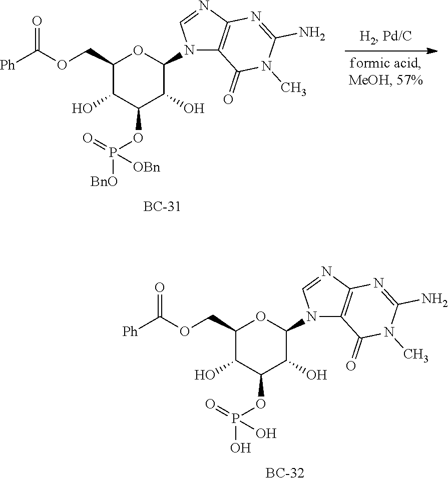

A suspension containing BC-31 (6.0 mg, 0.0087 mmol, 1.00 equiv.), Pd/C (14 mg, 10% w/w), formic acid (50 μL) and MeOH (2 mL) was sparged with Ar for 5 min. then switched to H₂, through which the suspension was rapidly stirred under for 3 hr. After sparging with Ar, the reaction mixture was filtered through celite using MeOH/H₂O and the collected filtrate was concentrated to dryness in vacuo, affording mgglu #52 (BC-32, 2.5 mg, 57%) which was deemed pure enough for no further purification steps. mgglu #52 was found to be identical to the major isomer peak on C18 in C. elegans wildtype (N2) by HPLC-HRMS (Method A) and MS². mgglu #52-HRMS (ESI) m/z: [M+H]⁺ calcd for C₁₉H₂₂N₅O₁₀P⁺512.1177; found 512.1158.

### Example 21. Step 1. 6′-O, 4′-O-TIPDSi-N⁹—(β-glucopyranosyl)-N¹-methylguanine (BC-33)

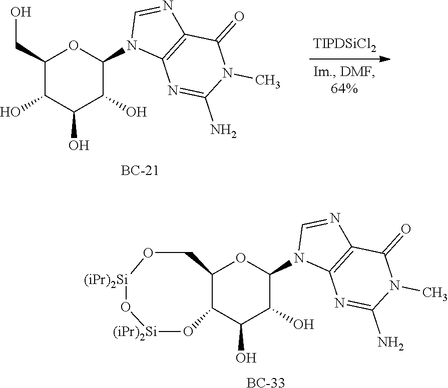

To a solution of BC-21 (70 mg, 0.214 mmol, 1.00 equiv.) in DMF (2 mL) at 0° C. was added TIPDSiCl₂ (120 μL, 0.376 mmol, 1.75 equiv.) and imidazole (66 mg, 0.970 mmol, 4.53 equiv.). The reaction mixture was stirred for 45 min at 0° C. and then diluted with DCM, followed by addition of H₂O. Organics were extracted 3× with DCM, combined, and then basified using sat. aq. NaHCO₃. Organics were then extracted from the aq. layer 3× with DCM, dried using MgSO₄, filtered, and concentrated in vacuo. Flash column chromatography on silica using a gradient of 5-40% MeOH in DCM was performed, affording BC-33 (77 mg, 64%) as a white solid. ¹H NMR (500 MHz, methanol-d₄): δ 7.86 (s, 1H), 5.40 (d, J=9.4 Hz, 1H), 4.16 (dd, J=12.7, 2.2 Hz, 1H), 3.98-3.88 (m, 3H), 3.59 (t, J=9.0 Hz, 1H), 3.47 (dt, J=9.5, 1.5 Hz, 1H), 3.46 (s, 3H), 1.25-0.96 (m, 28H). ¹³C NMR (126 MHz, methanol-d₄): δ 158.9, 156.2, 151.4, 137.6, 116.8, 84.3, 80.8, 78.3, 73.7, 70.4, 62.1, 28.8, 18.0, 17.8, 17.8, 17.8, 17.7, 17.7, 17.6, 14.9, 14.5, 14.0, 13.8.

### Example 21. Step 2. Compound BC-34

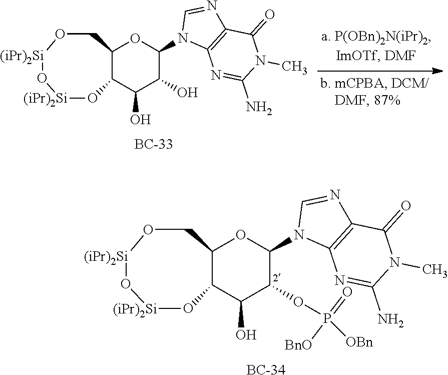

To a suspension of BC-33 (74 mg, 0.130 mmol, 1.00 equiv.) and DCM (1 mL)/DMF (3 mL) at room temp. was added dibenzyl N,N-diisopropylphosphoramidite (0.13 mL, 0.39 mmol, 3.00 equiv.), and ImOTf (85 mg, 0.39 mmol, 3.00 equiv.). The reaction mixture was stirred at room temp, for 15 min. at which a homogenous solution formed and then cooled to −78° C. after which 1 mL of DCM and mCPBA (77% max, 87 mg, 0.39 mmol, 3.00 equiv.) were added. The resulting mixture was stirred at −78° C. for 10 min, and was then quenched with the addition of sat. aq. NaHCO₃ (3 mL) followed by the addition of H₂O (10 mL) and DCM (20 mL). The organic layer was collected and the aqueous layer was extracted 3× with DCM (20 mL each). Combined organics were dried with MgSO₄, filtered, and concentrated in vacuo. Flash column chromatography on silica using a gradient of 2.5-20% MeOH in DCM was performed, affording BC-34 (94 mg, 87%). ¹H NMR (600 MHz, methanol-d₄): δ 7.88 (s, 1H), 7.33-7.20 (m, 8H), 7.08-7.02 (m, 2H), 5.68 (d, J=9.3 Hz, 1H), 4.93 (dd, J=11.5, 7.5 Hz, 1H), 4.87 (dd, J=11.1, 8.3 Hz, 1H), 4.68 (dd, J=11.7, 6.2 Hz, 1H), 4.58 (dd, J=11.7, 8.3 Hz, 1H), 4.17 (dd, J=12.8, 2.1 Hz, 1H), 3.99 (t, J=9.2 Hz, 1H), 3.93 (dd, J=12.9, 1.5 Hz, 1H), 3.87 (t, J=9.0 Hz, 1H), 3.54 (dt, J=9.5, 1.5 Hz, 1H), 3.26 (s, 3H), 1.27-0.97 (m, 28H). ¹³C NMR (126 MHz, methanol-d₄): δ 158.6, 156.1, 151.3, 137.6, 137.1 (d, J=7.1 Hz), 136.7 (d, J=7.1 Hz), 129.5, 129.4, 128.9, 128.3, 116.8, 82.3, 80.9, 80.1, 76.9, 70.9 (d, J=5.9 Hz), 70.6, 70.3 (d, J=5.3 Hz), 61.9, 28.8, 18.0, 17.8, 17.7, 17.6, 14.8, 14.5, 14.1, 13.8.

### Example 21. Step 3. Compounds BC-35 and BC-36

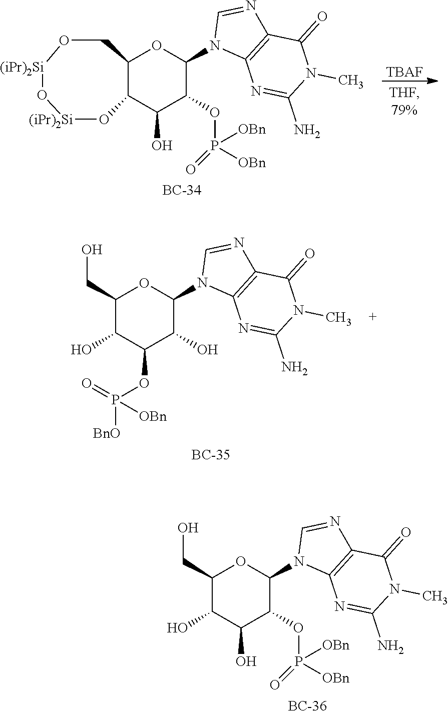

To a solution of BC-34 (96 mg, 0.116 mmol, 1.00 equiv.) in THF (4 mL) at 0° C. was added TBAF (1M in THF, 300 μL, 0.30 mmol, 2.59 equiv.). After 10 min., AcOH (100 μL) was added, and the resulting solution was concentrated in vacuo. Due to poor solubility of the resulting 3′-O product in DCM/MeOH, the crude was dissolved in a mixture of ACN and minimal H₂O and dry-loaded with 2 gram silica. Flash column chromatography on silica using a gradient of 5-40% MeOH in DCM was performed, affording pure 3′-O isomer (BC-35, 34 mg, 50%) and several mixed fractions containing 4 mg of 3′-O isomer (6%) and 16 mg of 2′-O isomer (BC-36, 23%). 2′-O isomer (BC-36): ¹H NMR (500 MHz, methanol-d₄): δ 7.99 (s, 1H), 7.38-7.20 (m, 8H), 7.10-6.99 (m, 2H), 5.69 (d, J=9.3 Hz, 1H), 4.97 (dd, J=11.8, 7.4 Hz, 1H), 4.92 (dd, J=11.9, 8.2 Hz, 1H), 4.66 (dd, J=11.9, 6.2 Hz, 1H), 4.53 (dd, J=11.9, 7.9 Hz, 1H), 3.89 (dd, J=12.4, 1.8 Hz, 1H), 3.80 (d, J=8.7 Hz, 1H), 3.74 (dd, J=12.1, 4.8 Hz, 1H), 3.65-3.51 (m, 2H), 3.26 (s, 3H). 2′-O isomer (BC-36): ¹³C NMR (126 MHz, methanol-d₄): δ 158.6, 156.2, 151.3, 137.1 (d, J=7.3 Hz), 136.7 (d, J=7.8 Hz), 129.6, 129.5, 129.3, 128.9, 128.1, 82.0, 81.3, 79.8, 77.3, 71.2, 70.9 (d, J=5.9 Hz, 1H), 70.21 (d, J=5.8 Hz, 1H), 62.2, 28.8. 3′-O isomer (BC-35): ¹H NMR (500 MHz, DMSO-d₆): δ 7.94 (s, 1H), 7.43-7.28 (m, 8H), 7.09 (s, 2H), 5.87 (d, J=6.6 Hz, 1H), 5.60 (d, J=7.3 Hz, 1H), 5.30 (d, J=9.3 Hz, 1H), 5.12 (d, J=6.9 Hz, 2H), 5.10-5.03 (m, 2H), 4.74 (t, J=5.9 Hz, 1H), 4.39 (q, J=8.9 Hz, 1H), 4.23 (td, J=9.2, 6.5 Hz, 1H), 3.72 (dd, J=11.2, 5.7 Hz, 1H), 3.60-3.47 (m, 2H), 3.43 (ddd, J=11.2, 5.8, 1.8 Hz, 1H), 3.32 (s, 3H). 3′-O isomer (BC-35): ¹³C NMR (126 MHz, DMSO-d₆): δ 156.4, 154.3, 149.7, 136.5 (d, J=3.6 Hz), 136.4 (d, J=3.8 Hz), 136.1, 128.4, 128.2, 128.1, 127.7, 115.5, 84.3 (d, J=6.7 Hz), 81.9, 79.4, 69.9, 68.5, 68.4, 68.3, 60.4, 28.1.

### Example 21. Step 4. mgglu #2 (BC-37)

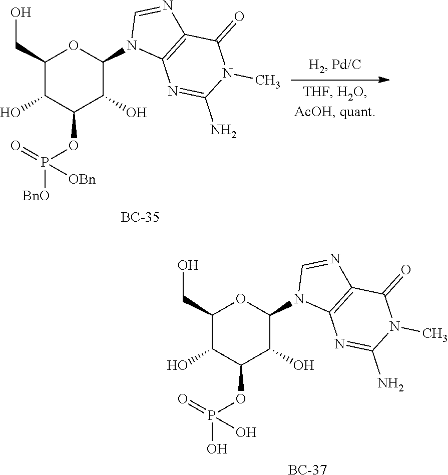 [SMILES: Cn1c(N)nc2c(ncn2[C@@H]2O[C@H](CO)[C@@H](O)[C@H](OP(=O)(OCc3ccccc3)OCc3ccccc3)[C@H]2O)c1=O ; "BC-35" ; Cn1c(N)nc2c(ncn2[C@@H]2O[C@H](CO)[C@@H](O)[C@H](OP(=O)(O)O)[C@H]2O)c1=O ; "BC-37"]

A suspension containing BC-35 (8 mg, mmol, 1.00 equiv.), Pd/C (13 mg, 10% w/w), AcOH (100 μL) H₂O/THF (4 mL, 1:1) was sparged with Ar for 5 min. then switched to H₂, through which the suspension was rapidly stirred under for hr. After sparging with Ar, the reaction mixture was filtered through celite using MeOH/H₂O and the collected filtrate was concentrated to dryness in vacuo affording mgglu #2 (BC-37, 5.5 mg, quant). mgglu #X was compared to the corresponding peak in C. elegans wildtype (N2) endo-metabolome samples by HILIC-HRMS. Chromatographic Method E was used.

### Example 22. Step 1. Compound BC-38

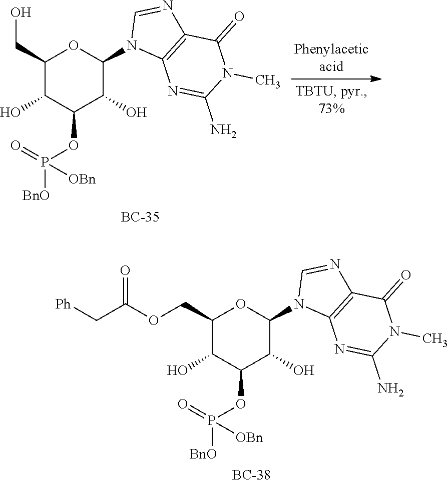

Phenylacetic acid (9.0 mg, 0.066 mmol, 2.64 equiv.) and TBTU (21 mg, 0.065 mmol, 2.60 equiv.) were added to a solution of BC-35 (15 mg, 0.025 mmol, 1.00 equiv.) in 1 mL dry pyridine. The resulting mixture was stirred for 4 hr at room temp., MeOH (1 mL) and DCM (2 mL) was added, the solution was transferred to a round-bottom flask, and then concentrated to dryness in vacuo ensuring all pyridine was removed. Flash column chromatography on silica using a gradient of 2.5-40% MeOH in DCM was performed, affording BC-38 (12.8 mg, 73%) as a white solid. ¹H NMR (500 MHz, methanol-d₄): δ 7.77 (s, 1H), 7.40-7.16 (m, 15H), 5.44 (d, J=9.4 Hz, 1H), 5.17-5.08 (m, 4H), 4.50 (dd, J=12.0, 1.9 Hz, 1H), 4.45 (q, J=8.7 Hz, 1H), 4.29 (dd, J=12.0, 5.6 Hz, 1H), 4.24 (t, J=9.3 Hz, 1H), 3.78 (ddd, J=10.0, 5.6, 2.2 Hz, 1H), 3.71 (t, J=9.4 Hz, 1H), 3.66 (d, J=15.2 Hz, 1H), 3.62 (d, J=15.2 Hz, 1H), 3.46 (s, 3H). ¹³C NMR (126 MHz, methanol-d₄): δ 173.2, 158.9, 156.2, 151.4, 137.8, 137.5 (d, J=2.6 Hz), 137.4 (d, J=2.6 Hz), 135.6, 130.4, 129.6, 129.5, 129.0, 128.0, 116.9, 85.7 (d, J=6.9 Hz), 85.6, 83.9, 77.8, 71.9 (d, J=3.6 Hz), 70.8, 70.1 (d, J=3.6 Hz), 64.4, 41.8, 28.8.

### Example 22. Step 2. mgglu #11 (BC-39)

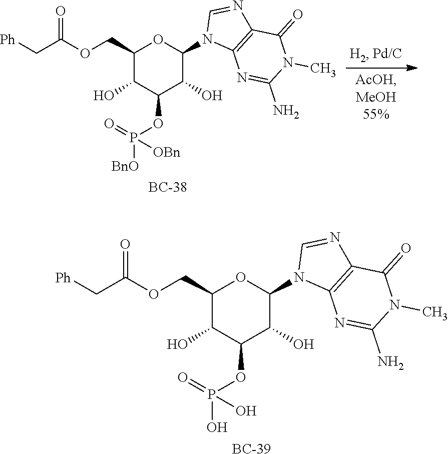

A suspension containing BC-38 (12.5 mg, 0.018 mmol, 1.00 equiv.), Pd/C (17 mg, 10% w/w), AcOH (117 μL) and MeOH (2.5 mL) was sparged with Ar for 5 min. then switched to H₂, through which the suspension was rapidly stirred under for 1.5 hr. After sparging with Ar, the reaction mixture was filtered through celite using MeOH/H₂O and the collected filtrate was concentrated to dryness in vacuo. The dried solution was loaded onto celite in H₂O and flash column chromatography on C18 using a gradient of 0-100% ACN (w/0.1% formic acid) in H₂O (w/0.1% formic acid) was performed, affording mgglu #11 (BC-39, 5.0 mg, 55%) of which was found to be identical to a minor isomer peak on C18 in C. elegans wildtype (N2) and fem-3 (OE) and him-5 endo-metabolome samples by HPLC-HRMS. Chromatographic Method B was used.

### Example 23. Step 1. 6′-O, 4′-O-TIPDSi-N⁹—(β-glucopyranosyl)-N²-methylguanine (BC-40)

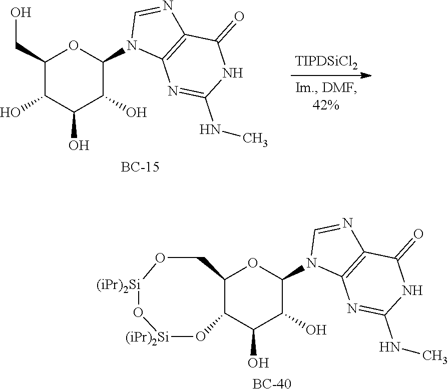

To a solution of BC-15 (95 mg, 0.290 mmol, 1.00 equiv.) in DMF (2 mL) at 0° C. was added TIPDSiCl₂ (140 μL, 0.435 mmol, 1.50 equiv.) and imidazole (93 mg, 1.36 mmol, 4.70 equiv.). The reaction mixture was stirred for 45 min at 0° C. and then diluted with DCM, followed by addition of H₂O. Organics were extracted 3× with DCM, combined, and then basified using sat. aq. NaHCO₃. Organics were then extracted from the aq. layer 3× with DCM, dried using Na₂SO₄, filtered, and concentrated in vacuo. Flash column in chromatography on silica using a gradient of 5-40% MeOH in DCM was performed, affording BC-40 (70 mg, 42%) as a white solid. ¹H NMR (600 MHz, methanol-d₄): δ 7.84 (s, 1H), 5.42 (d, J=9.3 Hz, 1H), 4.17 (dd, J=12.6, 2.1 Hz, 1H), 4.11 (t, J=9.2 Hz, 1H), 3.97-3.88 (m, 2H), 3.61 (t, J=9.0 Hz, 1H), 3.49 (d, J=9.4 Hz, 1H), 2.94 (s, 3H), 1.30-0.90 (m, 28H). ¹³C NMR (126 MHz, methanol-d₄): δ 159.5, 154.8, 153.5, 138.1, 117.4, 85.2, 80.8, 78.3, 73.36, 70.4, 62.1, 28.2, 18.0, 17.8, 17.8, 17.7, 17.6, 14.9, 14.5, 13.9, 13.8.

### Example 23. Step 2. Compound BC-41

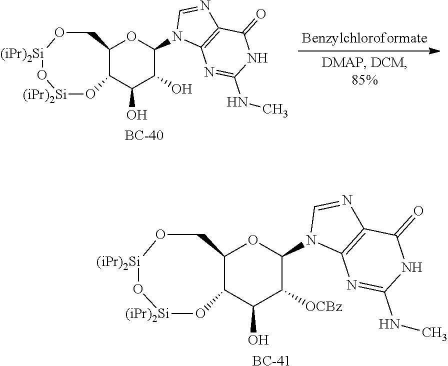

Benzylchloroformate (75 μL, 0.526 mmol, 4.28 equiv.) and DMAP (52.5 mg, 0.430 mmol, 3.41 equiv.) were added portion wise to a solution of BC-40 (72 mg, 0.126 mmol, 1.00 equiv.) in 4 mL DCM at 0° C. over a 45 min period. The resulting solution was stirred up to room temp, and stirred at that temp, for 15 min. The reaction mixture was then diluted with DCM and quenched with the addition of sat. aq. NaHCO₃. The organic layer was collected and additional organics were extracted 3× with DCM. The combined organics were dried using Na₂SO₄, filtered, and concentrated in vacuo. Flash column 20 chromatography on silica using a gradient of 3-30% MeOH in DCM afforded BC-41 (73 mg, 85%) as a white solid. ¹H NMR (600 MHz, methanol-d₄): δ 7.81 (s, 1H), 7.30-7.23 (m, 3H), 7.11-7.05 (m, 2H), 5.65 (d, J=9.4 Hz, 1H), 5.22 (br m, 1H), 5.05 (d, J=12.3 Hz, 1H), 4.91 (d, J=12.3 Hz, 1H), 4.18 (dd, J=12.8, 2.3 Hz, 1H), 4.01 (t, J=9.3 Hz, 1H), 3.95 (d, J=11.4 Hz, 1H), 3.86 (t, J=9.1 Hz, 1H), 3.57 (dt, J=9.5, 1.4 Hz, 1H), 2.89 (s, 3H), 1.24-1.00 (m, 28H). ¹³C NMR (126 MHz, methanol-d₄): δ 159.3, 155.7, 154.8, 153.1, 137.8, 136.7, 129.6, 129.5, 128.8, 117.1, 80.9, 78.6, 75.7, 70.7, 70.4, 62.0, 28.2, 18.0, 17.8, 17.7, 17.5, 14.8, 14.5, 14.0, 13.8.

### Example 23. Step 3. Compound BC-42

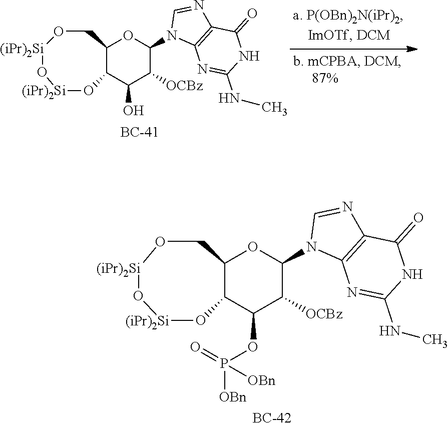

To an inhomogeneous solution of BC-41 (73 mg, 0.104 mmol, 1.00 equiv.) in DCM (3 mL) was added dibenzyl N,N-diisopropylphosphoramidite (70 μL, 0.208 mmol, 2.00 equiv.), and ImOTf (45 mg, 0.208 mmol, 2.00 equiv.). After 1 hr, an additional portion of dibenzyl N,N-diisopropylphosphoramidite (27.5 μL, 0.082 mmol, 0.78 equiv.), and ImOTf (11 mg, 0.050 mmol, 2.48 equiv.) were added and stirred for another hr. The resulting solution was cooled to 78° C. after which mCPBA (77% max, 95 mg, 0.425 mmol, 4.09 equiv.) was added. The resulting mixture was stirred up to 0° C. over a 1 hr period and was then diluted in DCM and quenched with the addition of sat. aq. NaHCO₃ (10 mL). The organic layer was collected and the aqueous layer was extracted 3× with DCM (15 mL each). Combined organics were dried with Na₂SO₄, filtered, and concentrated in vacuo. Flash column chromatography on silica using a gradient of 3-30% MeOH in DCM was performed, affording BC-42 (88 mg, 87%) as a white solid. ¹H NMR (600 MHz, methanol-d₄): δ 7.86 (s, 1H), 7.38-7.30 (m, 10H), 7.24-7.19 (m, 3H), 7.02-6.97 (m, 2H), 5.75 (d, J=8.9 Hz, 1H), 5.72-5.60 (br m, 1H), 5.02-4.95 (m, 4H), 4.94 (d, J=12.2 Hz, 1H), 4.76 (q, J=8.8 Hz, 1H), 4.66 (d, J=12.3 Hz, 1H), 4.24 (t, J=9.2 Hz, 1H), 4.17 (dd, J=12.9, 2.1 Hz, 1H), 3.98 (dd, J=12.9, 1.6 Hz, 1H), 3.64 (dt, J=9.5, 1.9 Hz, 1H), 2.90 (s, 3H), 1.16-0.87 (m, 28H). ¹³C NMR (126 MHz, methanol-d₄): 159.3, 155.2, 154.8, 153.0, 138.0, 137.1 (d, J=6.7 Hz), 136.9 (d, J=6.5 Hz), 136.5, 129.8, 129.7, 129.6, 129.5, 129.4, 129.3, 128.8, 82.2, 82.1, 80.1, 76.3, 71.2 (d, J=5.8 Hz), 71.0 (d, J=5.7 Hz), 70.9, 69.9 (d, J=5.0 Hz), 61.8, 28.2, 18.0, 17.9, 17.8, 17.7, 17.5, 14.4, 14.3, 14.2, 14.1. HRMS (ESI) m/z: [M+H]⁺ calcd for C₄₆H₆₃N₅O₁₂PSi₂⁺ 964.3744; found 964.3733.

### Example 23. Step 4. Compound BC-43

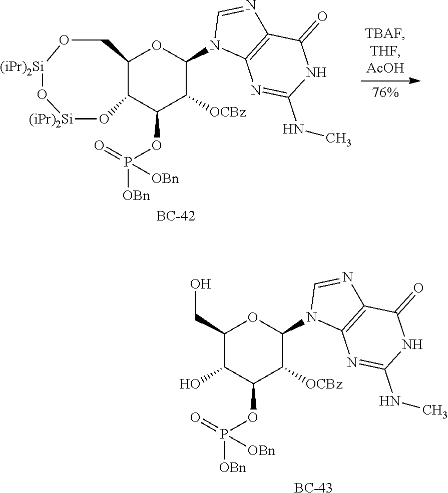

To a solution of BC-42 (88 mg, 0.091 mmol, 1.00 equiv.) in THF (3.5 mL) containing AcOH (15 μL, 0.26 mmol) at 0° C. was added TBAF (1M in THF, 270 μL, 0.270 mmol, 2.97 equiv.). After stirring for 5 hr cold (cooling with an ice-water bath), additional TBAF (50 μL, 0.050 mmol, 0.55 equiv) and AcOH (5 μL) was added, and the resulting solution stirred for an additional 2 hr up to room temp. until majority of starting material was consumed. An additional portion of AcOH was added (100 μL) and the solution was then concentrated to remove THF. Flash column chromatography on silica using a gradient of 5-40% MeOH in DCM was performed, affording BC-43 (48 mg, 76%). Samples contained residual TBAF fragments (10% w/w) which were unable to completely separate with subsequent purification. ¹H NMR (600 MHz, methanol-d₄): δ 7.97 (s, 1H), 7.42-7.26 (m, 10H), 7.25-7.17 (m, 3H), 7.00-6.94 (m, 2H), 5.78 (d, J=9.2 Hz, 1H), 5.55-5.44 (br m, 1H), 5.11 (d, J=7.6 Hz, 1H), 5.10 (d, J=7.9 Hz, 1H), 5.00 (d, J=7.6 Hz, 2H), 4.86 (d, J=12.3 Hz, 1H), 4.75 (q, J=9.0 Hz, 1H), 4.58 (d, J=12.3 Hz, 1H), 3.95-3.88 (m, 2H), 3.80 (dd, J=12.4, 5.1 Hz, 1H), 3.69 (ddd, J=9.8, 5.1, 2.1 Hz, 1H), 2.89 (s, 3H). ¹³C NMR (126 MHz, methanol-d₄): δ 159.3, 155.2, 154.9 153.0, 137.8, 137.2 (d, J=7.3 Hz), 137.1 (d, J=7.3 Hz), 136.3, 129.6, 129.5, 129.1, 128.9, 128.8, 116.9, 82.7 (d, J=6.8 Hz), 80.7, 76.8, 71.1 (d, J=6.0 Hz), 70.9, 69.7 (d, J=3.7 Hz), 61.9, 28.2.

### Example 23. Step 5. Compound BC-44

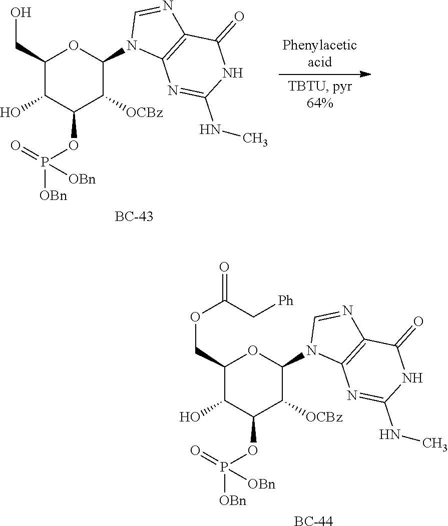

Phenylacetic acid (15 mg, 0.110 mmol, 4.78 equiv.) and TBTU (32 mg, 0.100 mmol, 4.35 equiv.) were added to a solution containing BC-43 (16.5 mg, 0.023 mmol, 1.00 equiv.) and 1 mL dry pyridine. The resulting mixture was stirred for 6 hr at room temp., DCM (2 mL) and MeOH (0.5 mL) were added, the solution was transferred to a round-bottom flask, and then concentrated to dryness in vacuo ensuring all pyridine was removed. Flash column chromatography on silica using a gradient of 2.5-30% MeOH in DCM was performed, affording BC-44 (12 mg, 64%) as a colorless solid. Samples contained some residual phenylacetic acid. ¹H NMR (600 MHz, methanol-d₄): δ 7.70 (s, 1H), 7.39-6.91 (m, 25H), 5.73 (d, J=9.2 Hz, 1H), 5.44-5.29 (br m, 1H), 5.10 (dd, J=7.9, 3.6 Hz, 2H), 4.99 (d, J=7.6 Hz, 2H), 4.85 (m, 1H), 4.73 (q, J=8.9 Hz, 1H), 4.58 (d, J=12.3 Hz, 1H), 4.53 (dd, J=12.3, 2.1 Hz, 1H), 4.33 (dd, J=12.2, 5.3 Hz, 1H), 3.91-3.86 (m, 1H), 3.84 (t, J=9.4 Hz, 1H), 3.68 (d, J=14.9 Hz, 1H), 3.64 (d, J=14.9 Hz, 1H), 2.89 (s, 3H).

### Example 23. Step 6. mgglu #31 (BC-45)

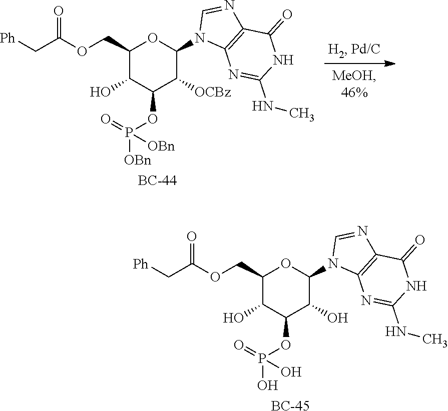

A suspension containing BC-44 (12 mg, 0.014 mmol, 1.00 equiv.), Pd/C (18 mg, 10% w/w), formic acid (200 μL) and MeOH (4 mL) was sparged with Ar for 5 min. then switched to H₂, through which the suspension was rapidly stirred under for 2 hr. After sparging with Ar, the reaction mixture was filtered through celite and washed with MeOH/H₂O and the collected filtrate was concentrated almost to dryness in vacuo and the resulting solution was loaded on celite. Flash column chromatography on C18 using a gradient of 1-100% ACN in H₂O (w/0.1% formic acid) afforded mgglu #31 (BC-45, 3.5 mg, 46%). mgglu #31 was found as an isomeric peak on C18 in C. briggsae endo-metabolome samples for m/z=526.1333 by HPLC-HRMS. Chromatographic Method B was used.

### Example 24 N⁹—(β-glucopyranosyl)-N⁶,N⁶-dimethyladenine (dmaglu #1, SI-2)

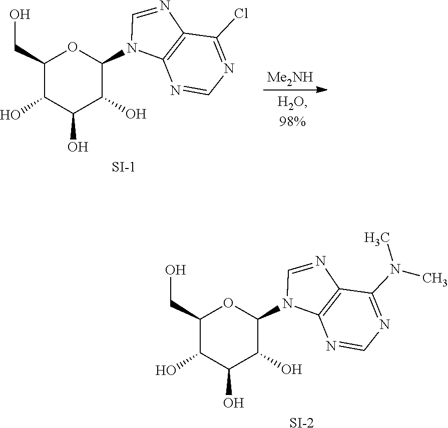

To SI-1 (50 mg, 0.158 mmol, 1.00 equiv.) in a high-pressure flask was added 40% NHMe₂ in H₂O (2.0 mL, 15.8 mmol, 100 equiv.). The flask was sealed and heated to 100° C., and the resulting solution was stirred for 8 hr. The solution was transferred to a round-bottom flask and concentrated to dryness in vacuo. The reaction mixture was then dissolved in MeOH/H₂O, silica gel (1 g) was added, and the mixture was concentrated to dryness in vacuo (for dry-loading). Flash column chromatography on silica using a gradient of 30-100% MeOH in DCM afforded SI-2 (50 mg, 98%) as a white solid. dmaglu #1 was compared to the corresponding peak in C. elegans, C. briggsae, and P. pacificus endo-metabolome samples on C18 and MS2 (Method A).

¹H NMR (500 MHz, DMSO-d₆): δ 8.33 (s, 1H), 8.22 (s, 1H), 5.43 (d, J=9.5 Hz, 1H), 5.36-5.28 (m, 2H), 5.18 (d, J=5.5 Hz, 1H), 4.59 (t, J=5.9 Hz, 1H), 3.96 (h, J=5.4 Hz, 1H), 3.69 (dd, J=10.6, 5.6 Hz, 1H), 3.59-3.36 (m, 9H), 3.25 (dt, J=9.0, 4.5 Hz, 1H). ¹³C NMR (126 MHz, DMSO-d₆): δ 154.2, 151.9, 150.7, 138.4, 119.1, 82.5, 80.0, 77.3, 71.3, 69.8, 60.8. Note: CH₃ signals were too broad to appear in the 13C spectrum.

HRMS (ESI) m/z: [M+H] calcd for C₁₃H₂₀O₅N₅ 326.1459; found 326.1459.

### Example 25 N⁷—(β-glucopyranosyl)-N⁶,N⁶-dimethyladenine (SI-4)

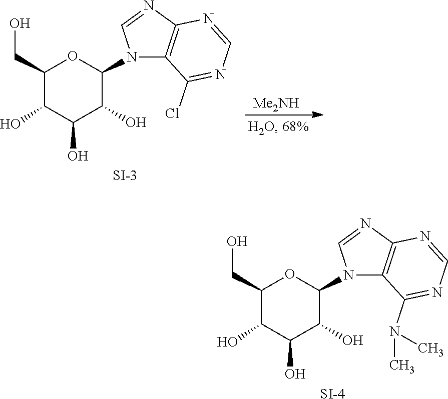

To SI-3 (88 mg, 0.181 mmol, 1.00 equiv.) in a high-pressure flask was added 40% NHMe2 in H₂O (2.5 mL, 19.8 mmol, 109 equiv.). The flask was sealed and heated to 100° C., and the resulting solution was stirred for 17 hr. The resulting solution was cooled, transferred to a round-bottom flask, and concentrated to dryness in vacuo. Flash column chromatography on silica using a gradient of 10-100% MeOH in DCM afforded SI-4 (40 mg, 68%). SI-4 was compared to the corresponding peak in C. elegans, C. briggsae, and P. pacificus endo-metabolome samples on C18.

¹H NMR (600 MHz, DMSO-d₆): δ 8.77 (s, 1H), 8.45 (s, 1H), 5.40 (d, J=9.4 Hz, 1H), 3.92 (t, J=9.0 Hz, 1H), 3.71 (d, J=10.2 Hz, 1H), 3.47-3.42 (m, 2H), 3.40 (t, J=8.8 Hz, 1H), 3.28 (1, J=8.9 Hz, 1H), 3.03 (s, 6H); ¹³C NMR (126 MHz, DMSO-d₆): δ 160.2, 154.9, 151.3, 145.6, 114.1, 84.2, 80.1, 77.2, 72.1, 69.7, 60.9, 40.4; HRMS (ESI) m/z: [M+H] calcd for C₁₃H₂₀O₅N₅ 326.1459; found 326.1459.

### Synthesis of Glucosyl Guanine Derivatives

### Example 26. N9-(β-glucopyranosyl)-N7-methylguanine (SI-14)

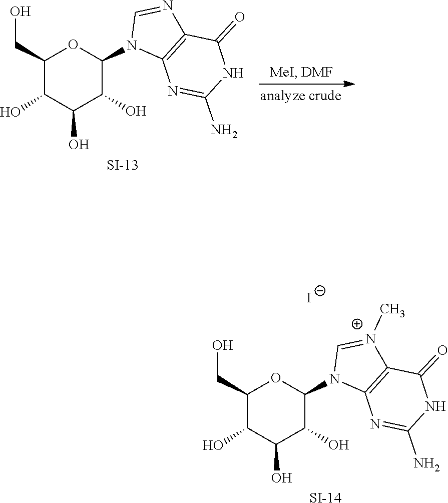

A solution of SI-13 (10 mg, 0.032 mmol, 1.00 equiv.) and Mel (8 μL, 0.128 mmol, 4.00 equiv.) in DMF (1 mL) was stirred for 48 hr at room temp. The resulting solution was concentrated in vacuo. Compound SI-14 was compared to corresponding peaks in C. elegans wildtype endo-metabolome samples by HILIC-HRMS.

¹H NMR (500 MHz, methanol-d₄): δ 9.42 (s, 1H), 5.61 (d, J=9.2 Hz, 1H), 4.16 (s, 3H), 3.91-3.84 (m, 2H), 3.75 (dd, J=12.1, 5.4 Hz, 1H), 3.63-3.58 (m, 1H), 3.58 (t, J=9.0 Hz, 1H), 3.52 (t, J=9.1 Hz, 1H); ¹³C NMR (126 MHz, methanol-d₄): δ 158.0, 155.7, 152.4, 139.1, 109.5, 85.7, 82.0, 78.5, 74.4, 71.2, 62.7, 37.5; HRMS (ESI) m/z: [M+Na]+ calcd for C₁₂H₁₇N₅O₆Na 350.1071; found 350.1060.

### Biological Examples

### Cells and Cell Culture

Human embryonic kidney 293 cells (ATCC) are propagated in DMEM supplemented with glutamine (Invitrogen) and 10% FCS (Hyclone, Ogden, Utah) (complete medium). In all cases herein, “293 cells” refers to human embryonic kidney (HEK) 293 cells. 293-hTLR3 cell line is generated by transforming 293 cells with pUNO-hTLR3. Cell lines 293-hTLR7, 293-hTLR8 and 293-hTLR9 (InvivoGen) are grown in complete medium supplemented with blasticidin (10 μg/ml) (Invivogen). Cell lines 293-ELAM-luc and TLR7-293 (M. Lamphier, Eisai Research Institute, Andover Mass.), and TLR3-293 cells are cultured as described (Kariko et al, 2004, mRNA is an endogenous ligand for Toll-like receptor 3. J Biol Chem 279:12542-12550). Cell lines 293, 293-hTLR7 and 293-hTLR8 are stably transfected with pTLR3-sh and selected with G-418 (400 μg/ml) (Invitrogen). Neo-resistant colonies are screened and only those that do not express TLR3, are determined as lacking IL-8 secretion in response to poly(I): (C), are used in further studies.

### Example 27

### Immunogeneicity of Modified RNA Materials as Measured by Stimulation of Human TLR3

Parental 293, 293-hTLR7 and 293-hTLR8 cells, all expressing TLR3-specific siRNA, and 293-hTLR9, TLR3-293 are seeded into 96-well plates (5×10⁴ cells/well) and cultured without antibiotics. On the subsequent day, the cells are exposed to modified RNAs described herein complexed to a delivery vehicle (e.g., Lipofectin® (Invitrogen)) as described (Kariko et al, 1998, ibid). Modified RNA as described herein is removed after one hour, and cells are further incubated in complete medium for 7 h. Supernatants are collected for IL-8 measurement.

To determine whether modification of nucleosides influences the modified RNA-mediated activation of TLRs, human embryonic kidney 293 cells are stably transformed to express human TLR3. The cell lines are treated with delivery vehicle (e.g., Lipofectin®)-complexed modified RNA, and TLR activation is monitored as indicated by interleukin (IL)-8 release.

### Example 28

### Immunogenicity of Modified RNAs as Measured by Stimulation of Human TLR7 and TLR8

To test whether 293 cells express endogenous TLR3 that interfere with assessing effects of modified RNA as described herein on specific TLR receptors, expression of endogenous TLR3 is eliminated from the 293-TLR8 cell line by stably transfecting the cells with a plasmid expressing TLR3-specific short hairpin (sh) RNA (also known as siRNA).

### Example 29

### Immunogenicity of Modified RNA as Mediated by TLR7 and TLR8 Signaling

To test the ability of modified RNAs described herein to stimulate TLR3, TLR7 and TLR8, RNA from different mammalian species are transfected into the TLR3, TLR7 and TLR8-expressing 293 cell lines described in the previous examples.

### Example 30

Translation of Proteins from Modified RNA In Vitro Materials and Experimental Methods
In Vitro Translation of mRNA in Rabbit Reticulocyte Lysate

In vitro-translation is performed in rabbit reticulocyte lysate (Promega, Madison Wis.). A 9-μL aliquot of the lysate is supplemented with 1 μL (1 μg) mRNA and incubated for 60 min at 30° C. An aliquot is removed for analysis using firefly and renilla assay systems (Promega, Madison Wis.), and a LUMAT LB 950 luminometer (Berthold/EG&G Wallac, Gaithersburg, Md.) with a 10 sec measuring time.

To determine the effect of modified RNAs translation efficiency in vitro, (0.1 μg/μL) uncapped modified mRNA encoding firefly luciferase is incubated in rabbit reticulocyte lysate for 1 h at 30° C., and luciferase activity is determined.

### Example 31

Enhanced Translation of Proteins from Modified RNAs in Cultured Cells Translation Assays in Cells

Plates with 96 wells are seeded with 5×10⁴ cells per well 1 day before transfection.

Lipofectin®-mRNA complexes are assembled and added directly to the cell monolayers after removing the culture medium (0.2 μm RNA-0.8 μg lipofectin in 50 μL per well). Cells are incubated with the transfection mixture for 1 h at 37° C., 5% CO₂ incubator, then the mixture is replaced with fresh, pre-warmed medium containing 10% FCS, then cells were analyzed as described in previous Examples.

To determine the effect of modified RNAs on translation in cultured cells, 293 cells are transfected with in vitro-transcribed, modified mRNA encoding the reporter protein renilla. Cells are lysed 3 h after initiation of transfection, and levels of renilla are measured by enzymatic assays.

Next, the experiment is performed with primary, bone marrow-derived mouse DC, in this case lysing the cells 3 h and 8 h after transfection.

### Example 32

Translation of Proteins from Modified RNA In Vivo

### Intracerebral Modified RNA Injections

All animal procedures are in accordance with the NIH Guide for Care and Use of Laboratory Animals and approved by the Institutional Animal Care and Use Committee. Male Wistar rats (Charles River Laboratories, Wilmington, MA) are anesthetized by intraperitoneal injection of sodium pentobarbital (60 mg/kg body weight). Heads are placed in a stereotaxic frame, and eight evenly spaced 1.5 mm diameter burr holes are made bilaterally [coordinates relative to bregma: anterior/posterior +3, 0, −3, −6 mm; lateral±2.5 mm] leaving the dura intact. Intra-cerebral injections are made using a 25 μL syringe (Hamilton, Reno, NV) with a 30 gauge, 1 inch sterile needle (Beckton Dickinson Labware, Franklin Lakes, N.J.) which is fixed to a large probe holder and stereotactic arm. To avoid air space in the syringe, the needle hub was filled with 55 μL complex before the needle is attached, and the remainder of the sample is drawn through the needle. Injection depth (2 mm) is determined relative to the surface of the dura, and 4 gl complex (32 ng modified RNAs described herein) is administered in a single, rapid bolus infusion. 3 hours (h) later, rats are euthanized with halothane, and brains are removed into chilled phosphate buffered saline.

Injection of Modified RNAs Described Herein into Mouse Tail Vein

Tail veins of female BALB/c mice (Charles River Laboratories) are injected (bolus) with 60 μL Lipofectin®-complexed modified RNAs described herein (0.26 μg). Organs are removed and homogenized in luciferase or Renilla lysis buffer in microcentrifuge tubes using a pestle. Homogenates are centrifuged, and supernatants are analyzed for activity.

### Delivery of Modified RNA to the Lung

Female BALB/c mice are anaesthetized using ketamine (100 mg/kg) and xylasine (20 mg/kg). Small incisions are made in the skin adjacent to the trachea. When the trachea is exposed, 50 μL of Lipofectin®-complexed modified RNA (0.2 μg) is instilled into the trachea toward the lung. Incisions are closed, and animals allowed to recover. 3 hours after modified RNA delivery, mice are sacrificed by cervical dislocation and lungs are removed, homogenized in luciferase or Renilla lysis buffer (250 μL), and assayed for activity. In a different set of animals, blood samples (100 μL/animal) are collected from tail veins, clotted, and centrifuged. Serum fractions are used to determine levels of TNF and IFNα by ELISA as described in the Examples above, using mouse-specific antibodies.

To determine the effect of modified RNA on translation in vivo, each hemisphere of rat brain cortexes was injected with modified RNA or unmodified RNA, and RNA translation was measured.

Next, expression studies are performed in mice. Firefly luciferase-encoding mRNAs because no endogenous mammalian enzyme interferes with its detection. Transcripts (unmodified and modified RNAs described herein) are constructed with cap, TEV (capTEVA50) and extended (~200 nt) poly(A) tails. 0.25 μg modified RNA Lipofectin®-complexed is injected into mice (intravenous (i.v.) tail vein). A range of organs are surveyed for luciferase activity to determine the optimum measurement site.

Translation efficiencies of conventional and modified RNA (0.015 mg/kg; 0.3 μg/animal given intravenously) are next compared in time course experiments.

In another experiment, 0.25 μg modified RNA-Lipofectin® is delivered to mouse lungs by intra-tracheal injection and surveyed for luciferase activity.

### Example 33

### Modified RNAs Stability In Vivo

To test whether in vivo protein production is quantitatively dependent on the concentration of intravenously-delivered mRNA, modified mRNAs are administered to mice at an appropriate amount (capTEVlucAn per animal) and spleens are analyzed 6 hours later as described above and luciferase activity is assessed.

What is claimed is:

1. A composition comprising RNA containing one or more covalently linked gluconucleosides.

2. The composition of claim 1, wherein the gluconucleoside comprises a moiety selected from a nucleobase, a nucleobase mimic or a nucleobase derivative linked to the anomeric position of an optionally substituted glucose.

3. The composition of claim 2, wherein the nucleobase, nucleobase mimic or nucleobase derivative is connected to the optionally substituted glucose via an N-glycosidic linkage.

4. The composition of any of claims 1 to 3, wherein the gluconucleoside is linked to the RNA via a phosphate, diphosphate, or polyphosphate linkage to any hydroxyl group of an optionally substituted glucose moiety.

5. The composition of claim 1, wherein the composition comprises a compound of formula I: 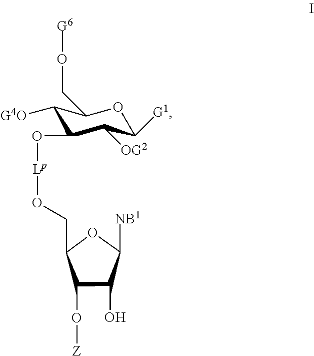
or a pharmaceutically acceptable salt thereof, wherein
G¹ is —OR¹⁰, —OC(O)R¹¹, or an optionally substituted group selected from the group consisting of 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 3- to 7-membered saturated or partially unsaturated monocyclic heterocyclyl having 1-3 heteroatoms independently selected from nitrogen, oxygen, and sulfur, 7- to 10-membered saturated or partially unsaturated bicyclic heterocyclyl having 1-4 heteroatoms independently selected from nitrogen, oxygen, and sulfur;
G² is selected from —H, —C(O)R, —C(S)R, —C(O)OR, —C(O)SR, —C(S)OR, —C(O)N(R)₂, —C(S)N(R)₂, or an optionally substituted group selected from C₁₋₃₂ aliphatic, C₁₋₃₂ heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
G⁴ is selected from —H, —C(O)R, —C(S)R, —C(O)OR, —C(O)SR, —C(S)OR, —C(O)N(R)₂, —C(S)N(R)₂, or an optionally substituted group selected from C₁₋₃₂ aliphatic, C₁₋₃₂ heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
G⁶ is selected from —H, —C(O)R, —C(S)R, —C(O)OR, —C(O)SR, —C(S)OR, —C(O)N(R)₂, —C(S)N(R)₂, or optionally substituted phosphate, diphosphate, triphosphate, or oligophosphate; a phosphate-, diphosphate-, triphosphate-, or oligophosphate-linked ribonucleotide; a phosphate-, diphosphate-, triphosphate-, or oligophosphate-linked RNA molecule; an acyl-linked amino acid, or peptide, or an optionally substituted group selected from C₁₋₃₂ aliphatic, C₁₋₃₂ heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
L^{P} is selected from: 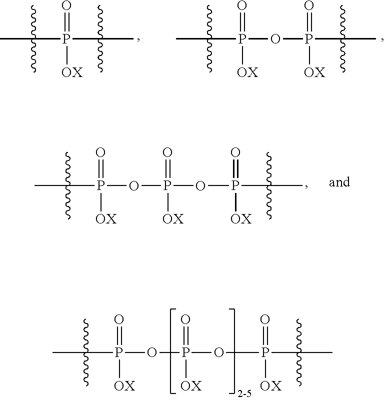
where X is independently selected at each occurrence from —H, C₁₋₄ aliphatic, a negative charge, and any metal atom or ion;
R¹⁰ and R¹¹ are each independently an optionally substituted group selected from the group consisting of: C₁₋₃₂ aliphatic, C₁₋₃₂ heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
each R is independently —H or an optionally substituted group selected from C₁₋₆ aliphatic, C₁₋₆ heteroaliphatic, 3- to 7-membered monocyclic cycloaliphatic, 3- to 7-membered saturated or partially unsaturated monocyclic heterocyclyl having 1-2 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or
two R, when attached to the same nitrogen atom, are taken together to form an optionally substituted 3- to 7-membered saturated or partially unsaturated ring having 0-1 additional heteroatoms independently selected from nitrogen, oxygen, and sulfur;
NB¹ is any nucleobase, including nucleobases that occur in RNA (e.g., any type of RNA from any organism), as well as non-natural nucleobase mimics and derivatives; and
Z is selected from —H; optionally substituted phosphate, diphosphate, or triphosphate; a ribonucleotide, and an RNA molecule.

6. The composition of claim 1, wherein the composition comprises a compound of formula II: 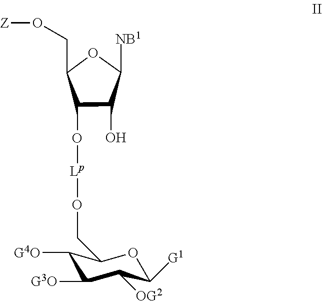
or a pharmaceutically acceptable salt thereof, wherein:
each of G¹, G², G⁴, L^{P}, NB¹, and Z is as defined for Formula I;
G³ is selected from —H, —C(O)R, or optionally substituted phosphate, diphosphate, triphosphate, or oligophosphate; a phosphate-, diphosphate-, triphosphate, or oligophosphate-linked ribonucleotide; a phosphate-, diphosphate-, triphosphate-, or oligophosphate linked RNA molecule; an acyl-linked amino acid, or an optionally substituted group selected from C₁₋₃₂ aliphatic, C₁₋₃₂ heteroaliphatic, phenyl, 8- to 10-membered aryl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; and
each R is independently-H or an optionally substituted group selected from C₁₋₆ aliphatic, C₁₋₆ heteroaliphatic, 3- to 7-membered monocyclic cycloaliphatic, 3- to 7-membered saturated or partially unsaturated monocyclic heterocyclyl having 1-2 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or
two R, when attached to the same nitrogen atom, are taken together to form an optionally substituted 3- to 7-membered saturated or partially unsaturated ring having 0-1 additional heteroatoms independently selected from nitrogen, oxygen, and sulfur.

7. The composition of claim 1, wherein the composition comprises a compound of formula III: 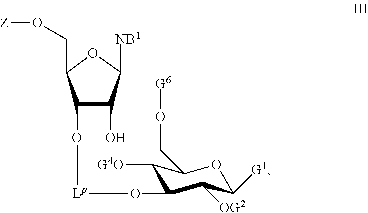
or a pharmaceutically acceptable salt thereof, wherein:
each of G¹, G², G⁴, G⁶, L^{P}, NB¹, and Z is as defined for Formula I.

8. The composition of claim 1, wherein the composition comprises a compound of formula IV: 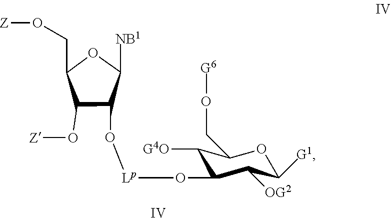
or a pharmaceutically acceptable salt thereof, wherein:
each of G¹, G², G⁴, G⁶, L^{P}, NB¹, and Z is as defined for Formula I; and
Z′ is selected from —H; optionally substituted phosphate, diphosphate, triphosphate, or oligophosphate; a ribonucleotide, and an RNA molecule.

9. The composition of claim 1, wherein the composition comprises a compound of formula V: 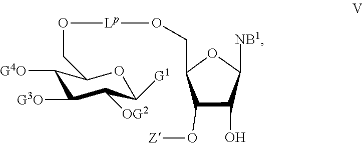
or a pharmaceutically acceptable salt thereof, wherein:
each of G¹, G², G³, G⁴, L^{P}, NB¹, are as defined for Formula I and Z′ is as defined for Formula IV.

10. The composition of claim 1, wherein the composition comprises a compound of formula VI: 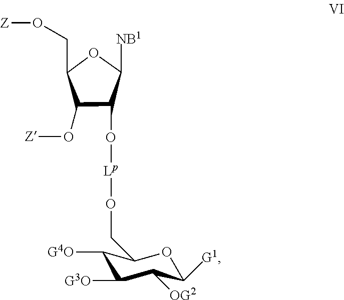
or a pharmaceutically acceptable salt thereof, wherein:
each of G¹, G², G³, G⁴, L^{P}, NB¹, Z, are as defined for Formula I and Z′ is as defined for Formula IV.

11. The composition of any one of claims 5-10, wherein G¹ is a N-linked nucleobase (e.g., linked through a N atom comprising part of the nucleobase structure).

12. The composition of any one of claims 5-10, wherein G¹ is substituted with an amine or an aminoalkyl group.

13. The composition of any one of claims 5-10, wherein R¹⁰ or R¹¹ is substituted with an amine or an aminoalkyl group.

14. The composition of any one of claims 5-13, wherein the moiety G¹ is selected from the group consisting of:

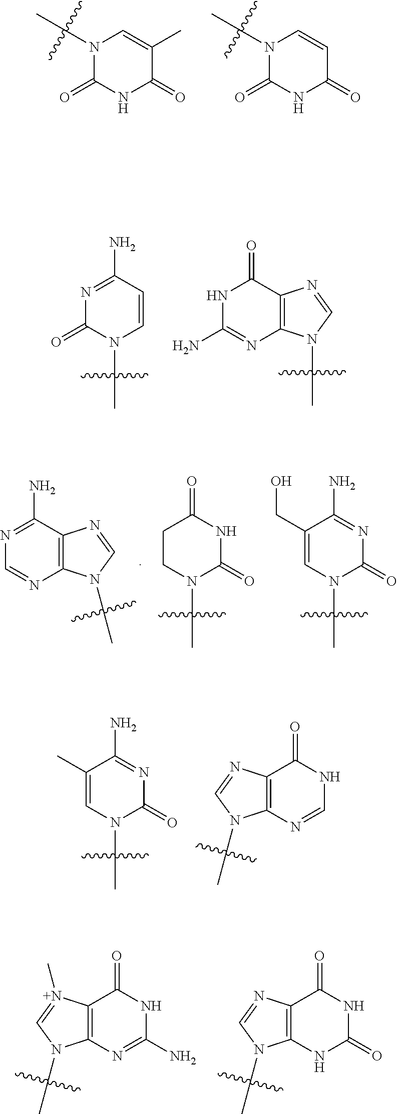

15. The composition of any one of claims 5-13, wherein the the moiety G¹ is selected from the group consisting of:

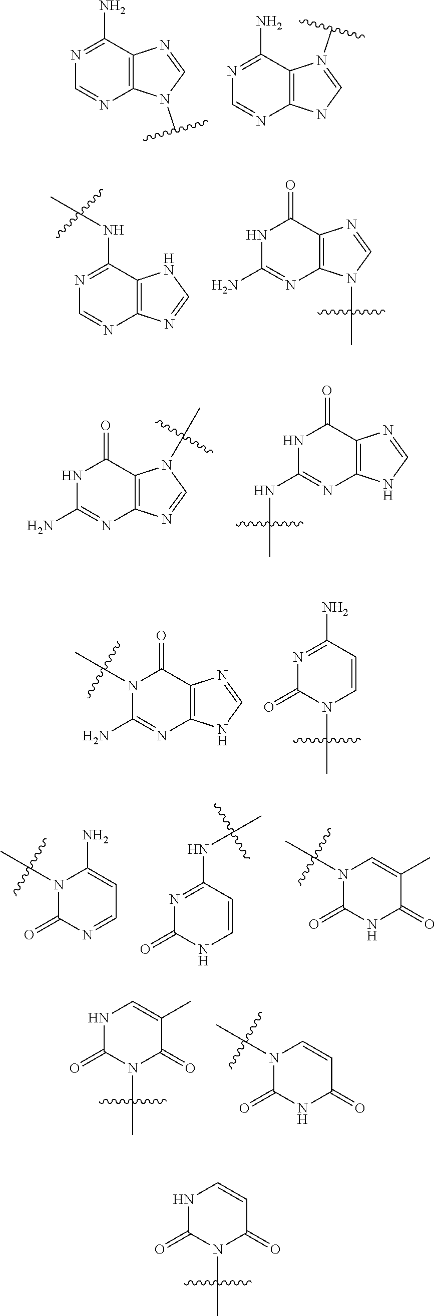

16. The composition of any one of claims 5-13, wherein the the moiety G¹ is selected from the group consisting of:

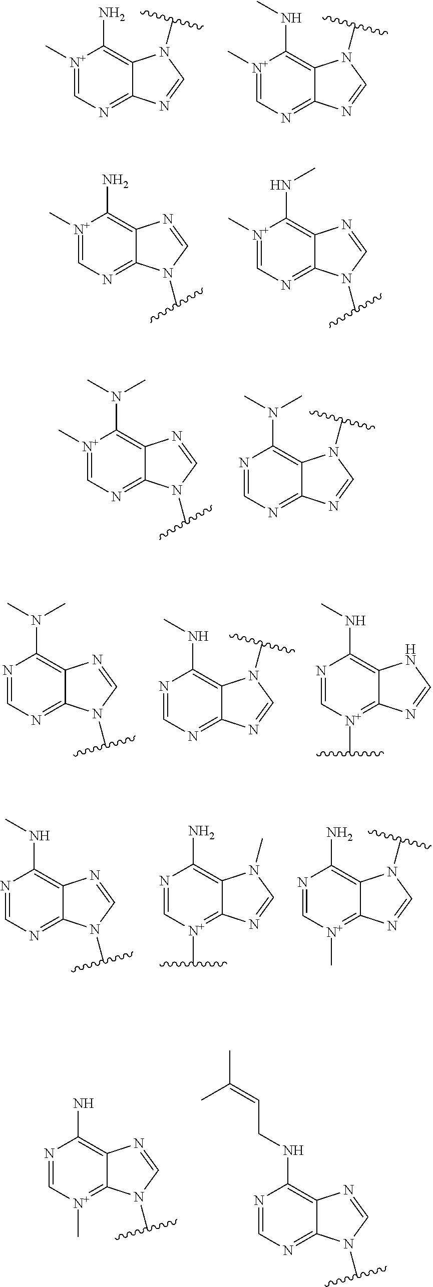

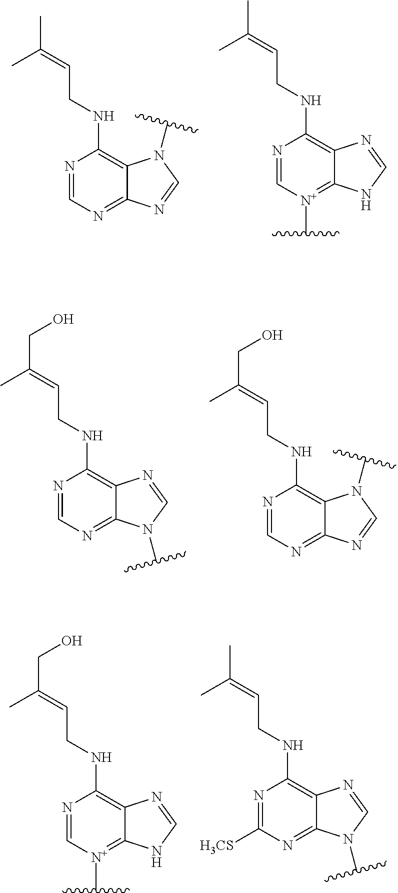

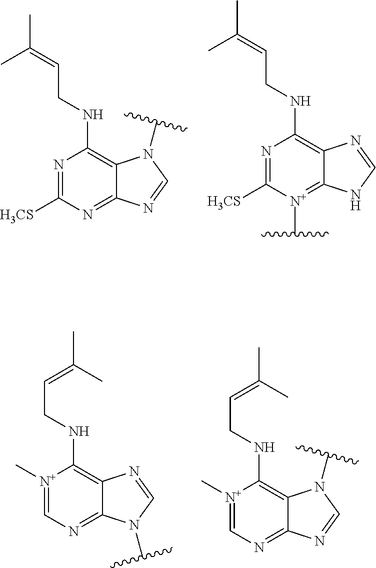

17. The composition of any one of claims 5-13, wherein the the moiety G¹ is selected from the group consisting of:

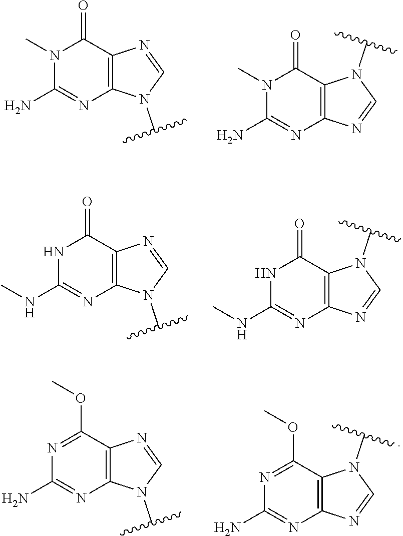 [SMILES: *n1cnc2c(=O)n(C)c(N)nc21 ; *n1cnc2nc(N)n(C)c(=O)c21 ; *n1cnc2c(=O)[nH]c(NC)nc21 ; *n1cnc2nc(NC)[nH]c(=O)c21 ; *n1cnc2c(OC)nc(N)nc21 ; *n1cnc2nc(N)nc(OC)c21]

18. The composition of any one of claims 5-13, wherein the the moiety G¹ is selected from the group consisting of:

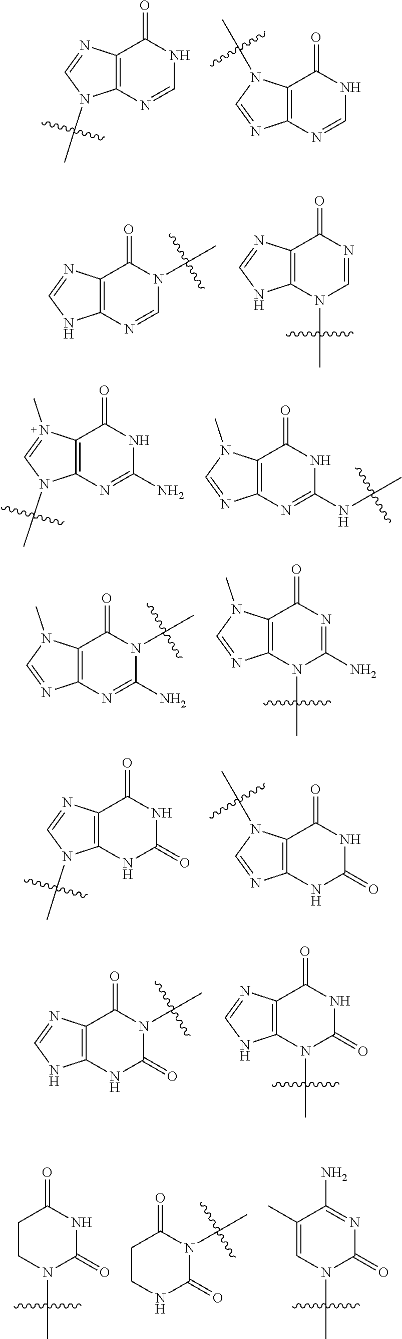

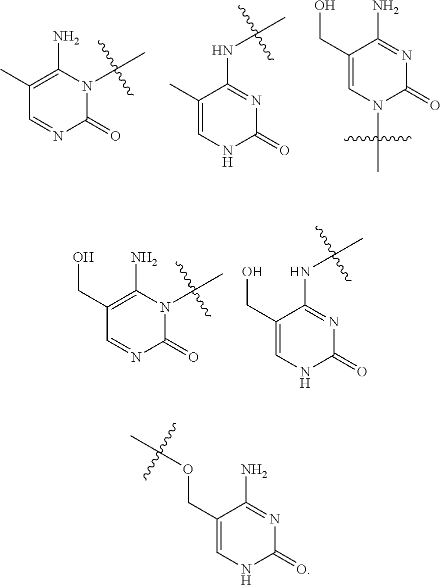

19. The composition of any of claims 5-15, wherein at least one of G² and G⁴ is —H.

20. The composition of claim 18, wherein G⁶ is other than —H.

21. The composition of any one of claims 5-21, wherein —Z or —Z′ is other than H.

22. The composition of claim 21, wherein —Z or —Z′ is a RNA molecule (e.g., an oligonucleotide or polynucleotide).

23. The composition of any of claims 1-22, wherein the gluconucleoside is attached to an end of the RNA.

24. The composition of claim 23, wherein the gluconucleoside is attached to the 3′ end of the RNA.

25. The composition of any one of the preceding claims, wherein the composition or gluconucleoside comprises a compound selected from a compound in Tables 1-6, or a pharmaceutically acceptable salt thereof.

26. A pharmaceutical composition comprising the composition of any one of claims 1-25, further comprising one or more pharmaceutically acceptable excipients.

27. A vehicle for administration of a composition of any one of claims 1-25 to a patient in need thereof.

28. A lipid nanoparticle comprising a composition of any one of claims 1-25.

29. A pharmaceutical composition comprising the lipid nanoparticle of claim 28, further comprising one or more pharmaceutically acceptable excipients.

30. A method of treating, curing, or ameliorating a health disorder in an animal or human comprising administering a therapeutically effective dose a composition comprising RNA containing one or more covalently linked gluconucleosides.

31. The method of claim 30, wherein the RNA containing one or more covalently linked gluconucleosides has organ-targeting properties different from a composition containing RNA lacking the covalently linked gluconucleosides.

32. The method of claim 30, wherein RNA in the therapeutic composition has enhanced stability or bioavailability relative to a composition containing RNA lacking the covalently linked gluconucleosides.

33. A method of delivering a therapeutic and/or prophylactic agent to a mammalian cell derived from a subject, the method comprising contacting the cell of the subject having been administered vehicle of claim 27, the lipid nanoparticle of claim 28, or the pharmaceutical composition of claim 29.

34. A method for treating a disease or a disorder in a subject in need thereof, the method comprising administering the vehicle of claim 27, the lipid nanoparticle of claim 28, or the pharmaceutical composition of claim 29 to the subject, wherein the composition is effective to treat the disease or disorder.

35. A method of producing a polypeptide of interest in a mammalian cell, the method comprising contacting the cell with the vehicle of claim 27, the lipid nanoparticle of claim 28, or the pharmaceutical composition of claim 29, wherein the composition comprises an mRNA, and wherein the mRNA encodes the polypeptide of interest, whereby the mRNA is capable of being translated in the cell to produce the polypeptide of interest.

36. A method for improving the efficacy of RNA-based vaccines, comprising formulating a vaccine comprising RNA containing one or more covalently linked gluconucleosides.

37. The method of claim 36, wherein the RNA-based vaccine is a vaccine against virus-caused infections.

38. The method of claim 36, wherein the RNA-based vaccine is useful for cancer immunotherapy.

39. A vaccine comprising RNA containing one or more covalently linked gluconucleosides characterized in that the vaccine has increased resistance to endogenous oligonucleotide-degrading enzymes.

40. The method of any one of claims 30-38, wherein the composition or gluconucleoside is any one of claims 1-25.

41. An adduct of ribonucleic acid and a MGN (e.g., as described in any one of Examples 1-26).

42. A method of increasing the stability of a ribonucleic acid, the method comprising the step of a attaching a MGN (e.g., as described in any one of Examples 1-26) to the ribonucleic acid (e.g., mRNA or mRNA-derivatives).

43. The method of claim 42, wherein the MGN is attached to the 5′- or 3′-ends of the ribonucleic acid (e.g., mRNA or mRNA-derivatives).
